(19) United States
(12) Patent Application Publication
(10) Pub. No.: US 2026/0026950 A1
(43) Pub. Date: Jan. 29, 2026

(54) SIDE BRANCH STENTING FOR TREATING A BIFURCATION

(71) Applicant: ADVANCED BIFURCATION SYSTEMS INC., Livermore, CA (US)

(72) Inventors: Ashur Bourang, Turlock, CA (US); Henry Bourang, Turlock, CA (US)

(21) Appl. No.: 18/781,494

(22) Filed: Jul. 23, 2024

(51) Int. Cl. A61F 2/954; A61F 2/958

(52) U.S. Cl. CPC A61F 2/954; A61F 2/958; A61F 2002/9583; A61F 2250/0067; A61F 2250/0098

(57) ABSTRACT

The present disclosure relates to a method for deploying a side branch stent in a bifurcated vessel that already contains a pre-existing main branch stent. The method involves inserting guide wires into both the main and side branches, and advancing a dual catheter delivery system, where only the side branch catheter carries the side branch stent. The system is retracted to position behind a side hole of the pre-existing stent, and guide wires are adjusted to reduce crossing. The catheters are aligned using marker bands and advanced towards the bifurcation until a specific tension is achieved. A balloon on the side branch catheter is then inflated to deploy the side branch stent through the side hole. This is followed by a kissing balloon inflation for optimal stent apposition. The catheters are then deflated and removed, leaving the stents aligned without gaps, ensuring effective treatment of the bifurcation.

### TECHNICAL FIELD AND BACKGROUND

In some examples, the present inventive subject matter relates to medical devices, and more particularly to stenting and treatment of bifurcated vessels.

In medical applications, a stent is an implantable scaffold that is typically delivered percutaneously and deployed in a vein, artery, or other tubular body organ for treating an occlusion, stenosis, aneurysm, collapse, dissection, or weakened, diseased, or abnormally dilated vessel or vessel wall. The stent is radially expanded in situ, thereby expanding and/or supporting the vessel wall or body organ wall. In particular, stents are quite commonly implanted in the coronary, cardiac, pulmonary, neurovascular, peripheral vascular, renal, gastrointestinal and reproductive systems, and have been successfully implanted in the urinary tract, the bile duct, the esophagus, the trachea-bronchial tree and the brain, to reinforce these body organs.

Stents are often used for improving angioplasty results by preventing elastic recoil and remodeling of the vessel wall and for treating dissections in blood vessel walls caused by balloon angioplasty of coronary arteries, as well as peripheral arteries, by pressing together the intimal flaps in the lumen at the site of the dissection. Conventional stents have been used for treating more complex vascular problems, such as lesions at or near bifurcation points in the vascular system, where a secondary artery branches out of a typically larger, main artery, with limited success rates.

Conventional stent technology is relatively well developed. Conventional stent designs typically feature a straight tubular, single-type cellular structure, configuration, or pattern that is repetitive through translation along the longitudinal axis. In many stent designs, the repeating structure, configuration, or pattern has strut and connecting balloon catheter portions that can impede blood flow at vessel bifurcations.

Furthermore, the configuration of struts and connecting balloon catheter portions may obstruct the use of post-operative devices to treat a daughter vessel in the region of a vessel bifurcation. For example, deployment of a first stent in the mother lumen may prevent a physician from inserting a daughter stent through the ostium of a daughter vessel of a vessel bifurcation in cases where treatment of the mother vessel is suboptimal because of displaced diseased tissue (for example, due to plaque shifting or “snow plowing”), occlusion, vessel spasm, dissection with or without intimal flaps, thrombosis, embolism, and/or other vascular diseases. A regular stent is designed in view of conflicting considerations of coverage versus access. For example, to promote coverage, the cell structure size of the stent may be minimized for optimally supporting a vessel wall, thereby preventing or reducing tissue prolapse. To promote access, the cell size may be maximized for providing accessibility of blood flow and of a potentially future implanted daughter stent to daughter vessels, thereby preventing “stent jailing,” and minimizing the amount of implanted material. Regular stent design has typically compromised one consideration for the other in an attempt to address both. Problems the present inventors observed involving daughter jailing, fear of plaque shifting, total occlusion, and difficulty of the procedure are continuing to drive the present inventors into the development of novel delivery systems, which are easier, safer, and more reliable to use for treating the above-indicated variety of vascular disorders. Although conventional stents are routinely used in clinical procedures, clinical data shows that these stents are not capable of completely preventing in-stent restenosis (ISR) or restenosis caused by intimal hyperplasia. In-stent restenosis is the reoccurrence of the narrowing or blockage of an artery in the area covered by the stent following stent implantation. Patients treated with coronary stents can suffer from in-stent restenosis.

Many pharmacological attempts have been made to reduce the amount of restenosis caused by intimal hyperplasia. Many of these attempts have dealt with the systemic delivery of drugs via oral or intravascular introduction. However, success with the systemic approach has been limited.

Systemic delivery of drugs is inherently limited since it is difficult to achieve constant drug delivery to the afflicted region and since systemically administered drugs often cycle through concentration peaks and valleys, resulting in time periods of toxicity and ineffectiveness. Therefore, to be effective, anti-restenosis drugs should be delivered in a localized manner. One approach for localized drug delivery utilizes stents as delivery vehicles. For example, stents seeded with transfected endothelial cells expressing bacterial betagalactosidase or human tissue-type plasminogen activator were utilized as therapeutic protein delivery vehicles. See, e.g., Dichek, D. A. et al., “Seeding of Intravascular Stents With Genetically Engineered Endothelial Cells,” Circulation, 80:1347-1353 (1989). U.S. Pat. No. 5,679,400, International Patent Publication No. WO 91/12779, entitled “Intraluminal Drug Eluting Prosthesis,” and International Patent Publication No. WO 90/13332, entitled “Stent With Sustained Drug Delivery,” which disclose stent devices capable of delivering antiplatelet agents, anticoagulant agents, antimigratory agents, antimetabolic agents, and other anti-restenosis drugs. U.S. Pat. Nos. 6,273,913; 6,383,215; 6,258,121; 6,231,600; 5,837,008; 5,824,048; 5,679,400; and 5,609,629 teach stents coated with various pharmaceutical agents such as Rapamycin, 17-beta-estradiol, Taxol and Dexamethasone. These and all other referenced patents are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

Therefore, given the challenges of current stent technology, a need exists for improved stent delivery systems and methods, particularly for treating bifurcated vessels. At least some of these objectives are addressed by the present inventive subject matter.

### BRIEF DESCRIPTION OF THE DRAWINGS

FIGS. 1A-1B illustrate an example of a system having an over-the-wire mother catheter and a rapid exchange daughter catheter.

FIGS. 2A-2B illustrate an example of a system having an over-the-wire daughter catheter and a rapid exchange mother catheter.

FIGS. 3A-3B illustrate an example of a system having a rapid exchange mother catheter and a rapid exchange daughter catheter.

FIGS. 4A-4B illustrate an example of a system having an over-the-wire mother catheter and an over-the-wire daughter catheter.

FIGS. 5A-5B illustrate another example of a system having a capture tube, an over-the-wire mother catheter, and a rapid exchange daughter catheter.

FIGS. 6A-6B illustrate another example of a system having a capture tube, an over-the-wire daughter catheter, and a rapid exchange mother catheter.

FIGS. 7A-7B illustrate another example of a system having a capture tube, a rapid exchange mother catheter, and a rapid exchange daughter catheter.

FIGS. 8A-8B illustrate another example of a system having a capture tube, an over-the-wire mother catheter, and an over-the-wire daughter catheter.

FIGS. 9A-9B illustrate yet another example of a system having a removable capture tube, an over-the-wire mother catheter, and a rapid exchange daughter catheter.

FIGS. 10A-10B illustrate yet other example of a system having a removable capture tube, an over-the-wire daughter catheter, and a rapid exchange mother catheter.

FIGS. 11A-11B illustrate yet another example of a system having a removable capture tube, a rapid exchange mother catheter, and a rapid exchange daughter catheter.

FIGS. 12A-12B illustrate yet another example of a system having a removable capture tube, an over-the-wire mother catheter, and an over-the-wire daughter catheter.

FIGS. 13A-13C illustrate still another example of a system having a snap fitting, an over-the-wire mother catheter, and a rapid exchange daughter catheter.

FIGS. 14A-14C illustrate still another example of a system having a snap fitting, an over-the-wire daughter catheter, and a rapid exchange mother catheter.

FIGS. 15A-15C illustrate still another example of a system having a snap fitting, a rapid exchange mother catheter, and a rapid exchange daughter catheter.

FIGS. 16A-16C illustrate still another example of a system having a snap fitting, an over-the-wire mother catheter, and an over-the-wire daughter catheter.

FIGS. 17A-17C illustrate another example of a system having a snap fitting, an over-the-wire mother catheter, and a rapid exchange daughter catheter.

FIGS. 18A-18C illustrate another example of a system having a snap fitting, an over-the-wire daughter catheter, and a rapid exchange mother catheter.

FIGS. 19A-19C illustrate another example of a system having a snap fitting, a rapid exchange mother catheter, and a rapid exchange daughter catheter.

FIGS. 20A-20C illustrate another example of a system having a snap fitting, an over-the-wire mother catheter, and an over-the-wire daughter catheter.

FIGS. 21A-21B illustrate yet another example of a system having an over-the-wire mother catheter and a rapid exchange daughter catheter.

FIGS. 22A-22B illustrate yet another example of a system having an over-the-wire daughter catheter and a rapid exchange mother catheter.

FIGS. 23A-23B illustrate yet another example of a system having a rapid exchange mother catheter and a rapid exchange daughter catheter.

FIGS. 24A-24B illustrate yet another example of a system having an over-the-wire mother catheter and an over-the-wire daughter catheter.

FIGS. 25A-25B, 26A-26B, 27A-27B, 28A-28B, 29A-29B, and 30A-30B illustrate an example of treating a bifurcation.

FIG. 31 illustrates an example of a stent.

FIG. 32 illustrates an example of a system having a mother catheter and a daughter catheter.

FIG. 33 highlights the distal portion of the system illustrated in FIG. 32.

FIG. 34 illustrates alignment of the stents in FIGS. 32-33.

FIG. 35 illustrates a cross-section of a stent crimped over a mother catheter and a daughter catheter.

FIG. 36 illustrates a stent disposed over a mother catheter and a daughter catheter.

FIG. 37 illustrates a stent disposed over a mother catheter and a daughter catheter, and a stent disposed over the daughter catheter.

FIGS. 38A-38M illustrate an example of treating a bifurcation.

FIGS. 39A-39M illustrate another example of treating a bifurcation.

FIGS. 40A-40H illustrate various stents that may be used with the systems and methods disclosed herein to treat bifurcations.

FIGS. 41A-41B illustrate examples of balloon configurations.

FIGS. 42A-42C illustrate engagement of a side branch stent with a main branch stent.

FIGS. 43A-43B illustrate other configurations of a side branch stent engaging a main branch stent.

FIGS. 44-46 illustrate still other configurations of engagement of a side branch stent with a main branch stent.

FIGS. 47A-47D illustrate interdigitation of a side branch stent and a main branch stent.

FIG. 48 illustrates another example balloon catheter.

FIGS. 49A-49D include schematic views illustrating aspects of twist resolution techniques of dual catheter systems described herein.

FIG. 50 is a pictorial view of a pocket, according to an example.

FIG. 51 illustrates example stents installed using example twist resolution techniques of dual catheter systems described herein.

FIGS. 52A-52L illustrate example techniques for side branch stenting through a previously deployed or preexisting main branch stent, according to some examples.

### DETAILED DESCRIPTION

The present inventive subject matter relates to delivery systems for delivery of stents to vessel bifurcations having a main branch and a side branch and is generally configured to at least partially cover a portion of the side branch as well as a portion of the main branch. However, this is not intended to be limiting, and one of skill in the art will appreciate that the devices and methods described herein may be used for treating other regions of the body.

The scientific community is slowly moving away from a main branch vs. side branch model and nomenclature. It is now well accepted that a “mother” vessel bifurcates into two “daughter vessels,” the two vessels that are anatomically after the carina. The vessel that appears to be the continuation of the mother vessel is usually less angulated. The other vessel is frequently smaller in diameter and may be commonly referred to as the side branch, or a daughter vessel. Therefore, in this specification, the terms “main branch,” “trunk,” or “mother vessel” may be used interchangeably. Also in this specification, the terms “side branch vessel” and “daughter vessel” may also be used interchangeably. The terms “main branch stent,” “trunk stent,” or “mother stent” are interchangeable, and the term “side branch stent” is also interchangeable with the term “daughter stent.” In the case where a main branch vessel bifurcates into two equally sized branches, one of the branches may still be considered to be the main branch or mother vessel, and the other branch may be considered a side branch or daughter vessel.

A variety of catheter designs may be employed to deploy and position the mother and daughter stents. Such catheters may be used in connection with multiple guidewires that terminate in the mother and daughter vessels. These guidewires may be used to facilitate introduction of the catheter, any angioplasty balloons, any stents, and/or to properly orient the stent or balloon within the vessel.

In general, the methods disclosed herein may utilize a catheter system comprising a catheter body having a mother vessel guidewire lumen and a daughter vessel balloon that is independently operable and coupled to the catheter body. The daughter balloon catheter portion has a daughter vessel guidewire lumen. The catheter system further includes a mother catheter balloon, and a stent is disposed over the balloon. The daughter catheter portion extends into the proximal opening of the mother stent and exits the mother stent through a side passage of the mother stent.

According to one method, a mother vessel guidewire is inserted into the mother vessel until a distal end of the mother vessel guidewire passes beyond the ostium of the daughter vessel, and a daughter vessel guidewire is inserted into the mother vessel until a distal end of the daughter vessel guidewire passes into the daughter vessel. To prevent the crossing of guidewires, the two vessels are wired through a guidewire catheter with two lumens to keep the guidewires separate and untangled.

The guidewire catheter is then removed, and a wire separator is placed on the wires to keep the guidewires unwrapped. The catheter system is then advanced over the mother and daughter vessel guidewires, with the mother and daughter vessel catheters passing over the mother vessel guidewire and the daughter vessel guidewire. The catheter system is advanced on both wires with the daughter vessel balloon catheter portion distal to the mother balloon catheter portion, leading the system. As the catheter system advances over the wires, the daughter vessel balloon will enter the daughter vessel and may be positioned after or simultaneously with placement of the mother vessel balloon. The mother balloon catheter portion of the catheter system is then advanced distally as far as it can be advanced where it is stopped by the carina. It cannot be advanced beyond the bifurcation site because the tension of the daughter catheter on the mother stent will prevent the mother catheter from moving distally. At this time, the distal portion of the mother stent is beyond the carina in the mother vessel and cannot be advanced any further. This method facilitates advancement of the catheter system to the bifurcation, which may be necessary for tortuous or calcified coronaries. Once the catheter system is in place, the daughter vessel balloon catheter portion is then pulled back relative to the mother catheter so that the proximal part of the daughter balloon is partially within the mother stent. Alignment can be performed with radiopaque markers, in that the proximal markers on the two balloons are next to each other. The operator can then gently push the catheter system distal to maximize apposition to the carina. The daughter balloon, which is now partially under the mother stent, is then inflated to ensure proper alignment of the mother stent. The daughter balloon may also have a stent on its distal portion, which would result in the proximal portion of the mother stent and the daughter stent to expand simultaneously. The daughter balloon is then deflated.

The mother balloon is then inflated, which deploys the mother stent. Kissing, or reinflation, of the two balloons is performed if necessary or for shifting plaque. The catheter system may be removed while the wires remain in place. In this example, or any of the other examples disclosed herein, an angioplasty catheter may be used to predilate the vessel and lesion prior to stenting. In some examples, primary stenting is employed where the stent is deployed without the predilation. The two vessels may be angioplastied separately if predilation is indicated on occasion.

In an alternative method, the mother catheter can be mounted on the daughter vessel guidewire and the daughter catheter can be mounted on the mother vessel guidewire. In daughter vessels with a high degree of angularity, for example, when the bifurcation angle is greater than about 60-70°, the friction between catheters is lower when the operator needs to draw the daughter stent proximally along the main branch and into the mother stent, as opposed to the prior configuration where the daughter stent is drawn along the side branch into the mother stent. The catheter system is advanced so the daughter balloon catheter leads the system and passes the ostium of the daughter vessel, while remaining in the mother vessel. As the catheter system is advanced further, the mother balloon catheter will enter the daughter vessel. The catheter system can only be advanced a certain distance toward the bifurcation, until it is stopped by the carina. It cannot be advanced beyond the bifurcation site because the tension of the daughter catheter on the mother stent will prevent the mother catheter from moving distally. At this time the distal portion of the mother stent is beyond the ostium of the daughter vessel and cannot be advanced any further. While the mother catheter is held in place, the daughter catheter is drawn back such that the proximal portion of the daughter balloon is partially in the mother stent. Alignment can be performed with radiopaque markers, in that the proximal markers on the two balloons are next to each other. The operator can then gently push the catheter system distally to maximize apposition to the carina. A stent on the daughter balloon (which is now partially under the mother stent) is aligned so that when the daughter balloon is inflated, the daughter stent and the proximal portion of the mother stent expand simultaneously and give complete coverage of the mother vessel. The daughter vessel balloon is then deflated. The mother vessel balloon is then inflated and the distal portion of the mother stent is expanded. A kissing procedure can also be performed if required.

The mother vessel can be stented if necessary with any commercially available stent. A balloon on a wire could be used as an alternative to the daughter catheter. In an alternative example, the catheter system can be arranged with the daughter balloon portion proximal to the mother balloon portion and advanced over the guidewires to the bifurcation. In the case of the mother catheter on the mother guidewire, the alignment of the mother stent with the ostium of the daughter vessel occurs because tension between the daughter guidewire and mother stent on the mother catheter prevents further advancement of the mother catheter. In the alternative case of the mother catheter on the daughter guidewire, the alignment of the mother stent with the ostium of the mother vessel occurs because tension between the mother guidewire and mother stent on the mother catheter (on the daughter guidewire) prevents further advancement of the mother catheter. In both cases the daughter stent is advanced into alignment with the mother stent and expanded. In some examples, the mother catheter is an over-the-wire (OTW) design and the daughter catheter is a rapid-exchange (RX) design with daughter catheter portion for example distal thereto. The daughter balloon is placed just distal to the tip of the mother catheter; this arrangement minimizes the overall profile of the catheter system and allows maximal tracking of the arteries. The system may additionally have stents crimped over the balloons. The daughter stent may be any length, but in some examples is approximately half the length of the daughter balloon or mother stent. The proximal end of the mother stent may be crimped only slightly to allow the daughter catheter balloon portion to operate independently so that it may be pushed or pulled without dislodging the mother stent.

An example comprises the following steps:
1. Advance the catheter system to bifurcation, daughter balloon catheter portion and mother balloon catheter portion in their respective vessels.
2. The mother catheter is no longer able to advance because of the tension between the mother stent and daughter catheter.
3. The daughter balloon proximal portion is drawn back into the mother stent and aligned with radiopaque markers.
4. While holding both the mother and daughter catheters tightly, the operator pushes forward lightly.
5. Inflate the daughter balloon and expand the daughter stent; approximately half of the daughter balloon distal portion will expand the “half-stent,” and half of the daughter balloon proximal portion will expand inside the mother vessel and partially expand the proximal portion of the mother stent. Expansion of the proximal portion of the mother stent and the daughter stent for example occur simultaneously.
6. Once the daughter stent is fully deployed, then the mother balloon can be fully expanded to deploy the distal portion of the mother stent.
7. A conventional kissing procedure may be utilized to ensure full apposition. In one particular aspect, the daughter balloon catheter portion may be used without a stent. This allows perfect alignment of the mother stent around the ostium of the daughter vessel. The daughter balloon would be used for the alignment as outlined in step three above and expands the proximal portion of the mother stent.

In an alternative example, the mother catheter is an over-the-wire (OTW) design and the daughter catheter is a rapid-exchange (RX) design with daughter catheter portion distal thereto. The system may additionally have stents crimped over the balloons. The daughter stent is for example less than the length of the mother balloon or stent, although this is not intended to be limiting, and the daughter stent may be any length. The proximal end of the mother stent may be partially crimped to allow the daughter catheter balloon portion to operate independently, so that it may be pushed or pulled without restriction and minimum friction, and without dislodging or affecting the mother stent. An example comprises the following steps:
1. Looping the OTW so that one operator can hold both guidewires with one hand and then push both catheters with the other.
2. Advance the catheter system to bifurcation, daughter balloon catheter portion and mother balloon catheter portion aligned in their respective vessels, as disclosed in steps two through three in the above example.
3. While holding both the mother and daughter catheters tightly, push the catheter system forward until the mother balloon catheter portion is stopped at the carina.
4. Inflate the daughter balloon and expand the daughter stent; approximately half of the daughter balloon distal portion will expand the “half-stent,” and half of the daughter balloon proximal portion will expand inside the mother vessel and partially expand the proximal portion of the mother stent.
5. Once the daughter stent is fully deployed, then the mother balloon can be fully expanded to deploy the distal portion of the mother stent.
6. A conventional kissing procedure may be utilized to ensure full apposition.

In one particular aspect, the daughter balloon catheter portion may be used without a stent. This would allow perfect alignment of the mother stent around the ostium of the daughter vessel. The daughter balloon would be used for the alignment as outlined in step three above and expand the proximal portion of the mother stent.

In an alternative example, the mother catheter is an over-the-wire design and the daughter catheter is a rapid-exchange design with daughter catheter portion distal thereto. The system may additionally have stents crimped over the balloons. The daughter stent may be approximately half the length of the mother balloon or stent, but this is not intended to be limiting, and the daughter stent may be any length. The proximal end of the mother stent may be partially crimped to allow the daughter catheter balloon portion to operate independently, so that it may be pushed or pulled without dislodging the mother stent. An example comprises the following steps:
1. Place the daughter catheter over the guidewire in the daughter vessel and slide the system into the guide catheter without placing the mother balloon over a guidewire at this time. After the leading daughter catheter enters the coronary artery and just before the mother catheter exits the guide catheter, insert the mother guidewire through the mother catheter and into the mother vessel, then push the system out of the guide catheter over the two guidewires. This method mitigates wire wrap.
2. Advance the catheter system to the bifurcation, daughter balloon catheter portion and mother balloon catheter portion aligned in their respective vessels.
3. Advance the catheter system to bifurcation, daughter balloon catheter portion and mother balloon catheter portion aligned in their respective vessels, as disclosed in step two in the above example. Pull the daughter catheter back until the proximal markers on both balloons are aligned.
4. Inflate the daughter balloon and expand the daughter stent; approximately half of the daughter balloon distal portion will expand the “half-stent,” and half of the daughter balloon proximal portion will expand inside the mother vessel and partially expand the proximal portion of the mother stent.
5. Once the daughter stent is fully deployed, then the mother balloon can be fully expanded to deploy the distal portion of the mother stent.
6. A conventional kissing procedure may be utilized to ensure full apposition. In one particular aspect, the daughter balloon catheter portion may be used without a stent. This would allow perfect alignment of mother stent around the ostium of the daughter vessel. The daughter balloon would be used for the alignment as outlined in step three above and expand the proximal portion of the mother stent.

In an alternative example the mother and daughter systems' balloons are aligned. This example could include the mother stent and daughter stent or either stent. When there is both a mother stent and a daughter stent, the daughter stent is for example shorter than the mother stent, although it may be any length, and in some examples is approximately half the length of the mother stent so that the daughter stent could be mounted on the distal half of the daughter balloon. Furthermore, the proximal portion of the daughter catheter shaft is positioned under the non-uniformly crimped mother stent. The dual stent arrangement reduces the profile compared to a full-length stent that covers the entire length of the daughter balloon.

The methods described herein could alternatively include the step of flushing the catheters and the guidewire port to assist with maneuverability. The methods described herein could alternatively include the step of a couple of snap-on couplers that lock the two catheters together. In another particular aspect, each balloon catheter portion may include at least one radiopaque marker. With such a configuration, separation of the markers may be conveniently observed using fluoroscopy to indicate that the balloon catheter portions have passed beyond the ostium and the daughter balloon catheter portion has passed into the daughter vessel, thus aligning the passage of the stent with the ostium of the daughter vessel. In another particular aspect, the catheter systems design is contemplated to cover combinations of rapid exchange and over the wire; for visualization purposes the hybrid versions are convenient because they are easier to distinguish while using fluoroscopy.

In another particular aspect, the proximal balloon may be differentially expandable, such that one end of the balloon may expand prior to the other end. In another particular aspect, the proximal balloon catheter portion may receive a stent that can be crimped under variable pressure to allow the distal balloon catheter portion freedom of movement.

In another particular aspect, a stent may be crimped over the proximal balloon catheter portion and the stent may be designed to deploy with variable profile to better oppose the patient anatomy.

In another particular aspect, the distal balloon catheter portion may be delivered via a pull-away or peel-away capture tube. All of the above examples may utilize mother vessel stents having any diameter, with diameter for example ranging from about 2.5 to about 5 millimeters, and daughter vessel stent having any diameter, for example ranging from about 2 to about 5 millimeters. The length of the stents may be any length, for example in the range of about 4 to about 40 millimeters. The position of a stent on a catheter need not be fixed and may be positioned on either or both catheters.

### Catheter Configurations:

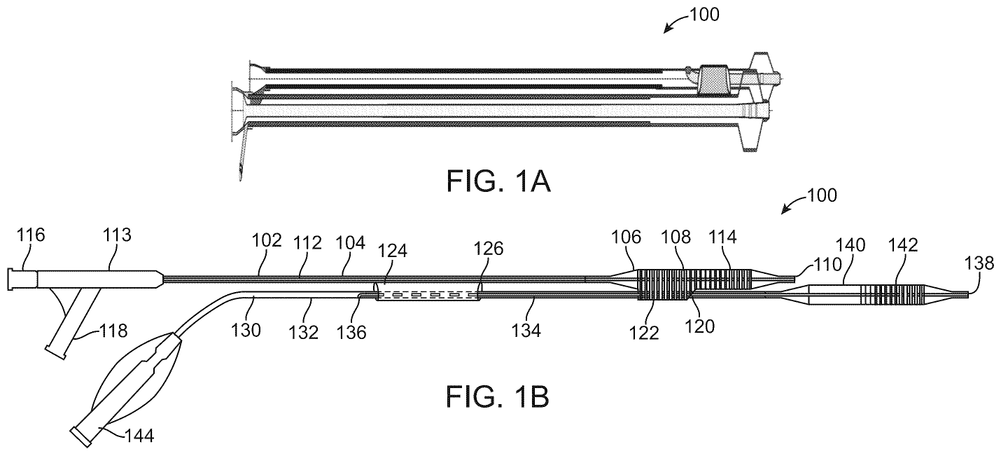

FIG. 1A illustrates an example of the catheter system 100 with a distal daughter balloon catheter portion comprising a balloon with a daughter stent crimped thereon. The daughter stent may be shorter than the mother stent, and it may not be centered on its corresponding balloon in this as well as any other examples disclosed herein. Thus, in some examples, a proximal portion of the daughter balloon remains uncovered by a stent, as will be discussed in greater detail below. In a particular example the daughter stent is for example about half the length of the mother stent. The distal daughter stent is crimped under standard conditions known in the art. The proximal mother balloon catheter portion comprises a mother balloon and a mother stent. The mother stent is crimped differentially along the longitudinal direction and circumferentially. In this example, the distal half of the mother stent is crimped under typical conditions to ensure that the mother stent is not dislodged during the alignment with the distal daughter balloon. Further, the proximal portion of the mother stent is crimped under non-standard, relatively loose, conditions to allow the distal daughter balloon catheter portion freedom of movement even though a portion of the daughter balloon catheter portion is circumferentially enclosed. The mother and daughter catheters are slidably attached to each other via a hollow exchange port. The exchange port is embedded in the side of the mother over the wire catheter and has an inner diameter just large enough to allow the insertion of the rapid exchange daughter catheter and balloon. The exchange port may be any length that extends between a proximal portion of the balloons and a distal portion of the catheter connectors, and in this example is about 10 centimeters long, but in some examples varies from about 1 centimeter to about 30 centimeters, and in more some examples is about 5 cm to about 10 cm long. The entry for the daughter catheter on the exchange port is proximal and the exit for the daughter catheter is on the distal end of the exchange port. The daughter catheter is loaded through the exchange port and the daughter balloon extends distally from the exit of the exchange port, for example about 5 centimeters. However, it is possible to have the exchange port any distance from the mother balloon, but for example about 1 to about 30 centimeters proximal to the mother balloon. The daughter stent can be crimped on to the balloon after it has been loaded through the exchange port. The exchange port for example has a tight fit to reduce catheter profile and for example has low friction to allow the operator to easily slide the catheters relative to each other.

FIG. 1B more clearly illustrates the features of the catheter system 100 in FIG. 1A. The catheter system 100 includes a first catheter 102 and a second catheter 130. The first catheter 102 includes an elongate shaft 104 with a radially expandable balloon 106 disposed near a distal end of the elongate shaft 104. A stent 108 having a proximal portion 122, a distal portion 114 and a side hole 120 is disposed over the balloon 106. The distal portion 114 is crimped to the balloon 106 to prevent ejection during delivery, while the proximal portion 122 is partially crimped to the balloon 106 so the second catheter 130 may be slidably advanced or retracted under the proximal portion 122 of stent 108. The first catheter 102 is an over-the-wire (OTW) catheter having a guidewire lumen 112 extending from the distal guidewire port 110 at the distal end of the elongate shaft 104 to the proximal end of the elongate shaft 104 into Y-adapter 113 having a connector 116. The connector 116 is for example a Luer connector, and this allows easy coupling with a syringe or other device for lumen flushing or injecting contrast media. When unconnected, the guidewire lumen 112 exits via connector 116. A second connector 118, also for example a Luer connector, allows attachment of an Indeflator or other device to the catheter 102 for inflation of the balloon 106 via an inflation lumen (not shown) in the elongate shaft 104. The first catheter 102 also includes a hollow exchange port tube 124 coupled to the elongate shaft 104. The hollow exchange port tube 124 may be coextruded with the elongate shaft 104, or it may be bonded or otherwise attached thereto using techniques known to those skilled in the art. The hollow exchange port 124 may alternatively be coupled with the elongate shaft 132 of the second catheter 130. The hollow exchange port tube 124 includes a central channel 126 extending therethrough and is sized to slidably receive a portion of the second catheter 130. Radiopaque markers may be placed at different locations along the shaft 104, often near the balloon 106 and/or stent 108, to help mark the proximal and distal ends of the stent or balloon, as well as to facilitate alignment of the two catheters during stent deployment, as discussed elsewhere in this specification.

The second catheter 130 includes an elongate shaft 132 with a radially expandable balloon 140 disposed near a distal end of the elongate shaft 132. A stent 142 is disposed over balloon 140. The stent 142 may have a length that matches the working length of the balloon 140, or the stent length may be shorter than the balloon working length. In some examples, the stent 142 is shorter than the working length of the balloon 140 so that a proximal portion of the balloon 140 is unconstrained by the stent 142, and this unconstrained portion of the balloon 140 may be slidably advanced or retracted through side hole 120 and under proximal portion 122 of stent 108 as will be discussed below. Stent 142 is crimped to balloon 140 to prevent ejection during delivery. At least a portion of balloon 140 and stent 142 are distally offset relative to balloon 106 and stent 108 so as to minimize profile of the device. In this example, the distal stent 142 may be deployed in a main branch of the vessel and the other stent 108 may be deployed in a side branch of the vessel. Alternatively, the distal stent 142 may be deployed in a side branch of a vessel and the other stent 108 may be deployed in the main branch of a vessel.

The second catheter 130 is a rapid exchange catheter (RX) having a guidewire lumen 134 extending from the distal guidewire port 138 at the distal end of the elongate shaft 132 to a proximal guidewire port 136, which is closer to the distal guidewire port 138 than the proximal end of the catheter shaft 132. The proximal guidewire port 136 is also unobstructed by the hollow exchange port tube 124 and for example proximal thereto. A connector 144, for example a Luer connector, is connected to the proximal end of the elongate shaft 132 and allows an Indeflator or other device to be coupled with an inflation lumen (not shown) in elongate shaft 132 for inflation of balloon 140. A portion of shaft 132 is disposed in the central channel 126 of the hollow exchange port tube 124, and this helps keep the two catheter shafts 104, 132 parallel and prevents tangling during delivery and as shaft 132 is slidably advanced or retracted relative to shaft 104. Also, another portion of shaft 132 is disposed under proximal portion 122 of stent 108. The second catheter 130 may also be slidably advanced or retracted under the proximal portion 122 of stent 108 so that the shaft 132 passes through the side hole 120 in stent 108. Radiopaque markers may be placed at different locations on the shaft 132, often near the balloon 140 or stent 142, to help mark the proximal and distal ends of the stent 142 or balloon 140, as well as to facilitate alignment of the two catheters during stent deployment, as discussed elsewhere in this specification.

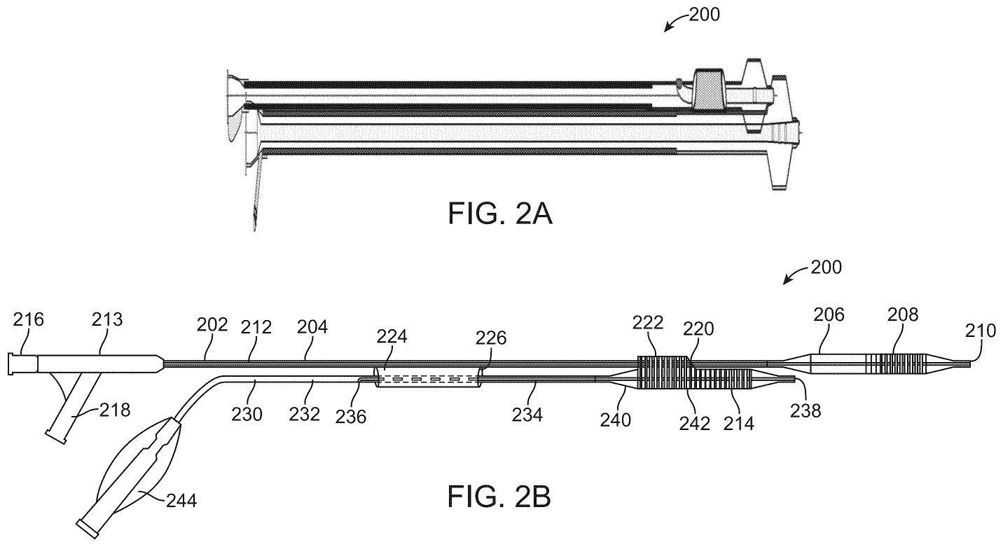

FIG. 2A illustrates a cross-sectional view of one example of a catheter system 200 with the daughter catheter balloon portion distal to the mother balloon portion utilizing the same exchange port as described in FIG. 1A. The mother balloon is for example at least about 5 centimeters distal from the exit of the exchange port. As disclosed above, the mother balloon could be distal from the exchange port from about 1 cm to about 30 centimeters.

FIG. 2B more clearly illustrates the features of the catheter system 200 in FIG. 2A. The catheter system 200 includes a first catheter 202 and a second catheter 230. The first catheter 202 includes an elongate shaft 204 with a radially expandable balloon 206 disposed near a distal end of the elongate shaft 204, and a stent 208 disposed over the balloon 206. The stent 208 may be the same length as the working length of the balloon 206, or it may be shorter. In some examples, the stent 208 is shorter than the working length of balloon 206 such that a proximal portion of balloon 206 remains unconstrained by stent 208. The proximal portion of balloon 206 may be slidably advanced and retracted under stent 242 via side hole 220. Stent 208 is crimped to the balloon 206 to prevent ejection during delivery. The first catheter 202 is an over-the-wire (OTW) catheter having a guidewire lumen 212 extending from the distal guidewire port 210 at the distal end of the elongate shaft 204 to the proximal end of the elongate shaft 204 into Y-adapter 213 having a connector 216. The connector 216 is for example a Luer connector, and this allows easy coupling with a syringe or other device for lumen flushing or injecting contrast media. When unconnected, the guidewire lumen 212 exits via connector 216. A second connector 218, also for example a Luer connector, allows attachment of an Indeflator or other device to the first catheter 202 for inflation of the balloon 206 via an inflation lumen (not shown) in the elongate shaft 204. The first catheter 202 also includes a hollow exchange port tube 224 coupled to the elongate shaft 204. The hollow exchange port tube 224 may be coextruded with the first shaft 204, or it may be bonded or otherwise attached thereto using techniques known to those skilled in the art. The hollow exchange port tube 224 may alternatively be coupled with the other shaft 232. The hollow exchange port tube 224 includes a central channel 226 extending therethrough and is sized to slidably receive a portion of the second catheter 230. Radiopaque markers may be placed at different locations along the shaft 204, often near the balloon 206 and/or stent 208, to help mark the proximal and distal ends of the stent 208 or balloon 206, as well as to facilitate alignment of the two catheters during stent deployment, as discussed elsewhere in this specification.

The second catheter 230 includes an elongate shaft 232 with a radially expandable balloon 240 disposed near a distal end of the elongate shaft 232. A stent 242 having a proximal portion 222, a distal portion 214, and a side hole 220 is disposed over balloon 240. The distal portion 214 is crimped to balloon 240 to prevent ejection during delivery, while the proximal portion 222 is partially crimped to balloon 240 so elongate shaft 204 may be slidably advanced or retracted under the proximal portion 222 of stent 242. The stent 242 may for example have a length that matches the working length of the balloon 240, or the stent length may be shorter than the balloon working length. At least a portion of balloon 206 and stent 208 are distally offset relative to balloon 240 and stent 242 so as to minimize profile of the device. In this example the distal stent 208 may be deployed in a main branch of the vessel and the other stent 242 may be deployed in a side branch of the vessel. Alternatively, the distal stent 208 may be deployed in a side branch of a vessel and the other stent 242 may be deployed in the main branch of a vessel.

The second catheter 230 is a rapid exchange catheter (RX) having a guidewire lumen 234 extending from the distal guidewire port 238 at the distal end of the elongate shaft 232 to a proximal guidewire port 236, which is closer to the distal guidewire port 238 than the proximal end of the catheter shaft 232. The proximal guidewire port 236 is also unobstructed by the hollow exchange port tube 224 and for example proximal thereto. A connector 244, for example a Luer connector, is connected to the proximal end of the elongate shaft 232 and allows an Indeflator or other device to be coupled with an inflation lumen (not shown) in elongate shaft 232 for inflation of balloon 240. A portion of shaft 232 is disposed in the central channel 226 of the hollow exchange port tube 224 and this helps keep the two catheter shafts 204, 232 parallel and prevents tangling during delivery and as shaft 232 is slidably advanced or retracted relative to shaft 204. Also, a portion of shaft 204 is disposed under proximal portion 222 of stent 242. The first catheter 202 may be slidably advanced or retracted under the proximal portion 222 of stent 242 so that the shaft 204 passes through the side hole 220 in stent 242. Radiopaque markers may be placed at different locations on the shaft 232, often near the balloon 240 or stent 242, to help mark the proximal and distal ends of the stent 242 or balloon 240, as well as to facilitate alignment of the two catheters during stent deployment, as discussed elsewhere in this specification.

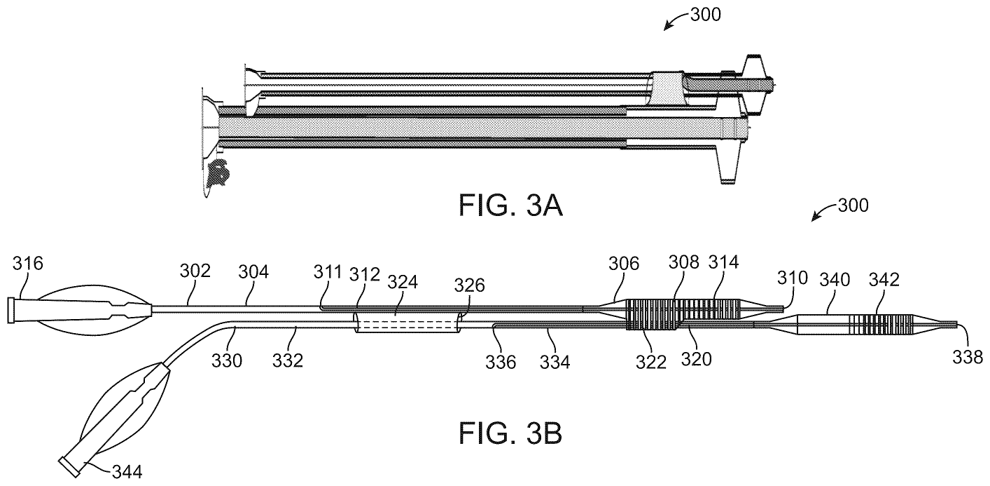

FIG. 3A illustrates a cross-sectional view of one example of a catheter system 300 with the mother and daughter catheters both having a rapid exchange design. In this particular example one of the catheters has a hollow exchange port embedded in its side and the other catheter is loaded through the exchange port. Typically, the catheter is loaded prior to having a stent crimped over the balloon portion.

FIG. 3B more clearly illustrates the features of the catheter system 300 in FIG. 3A. The catheter system 300 includes a first catheter 302 and a second catheter 330. The first catheter 302 includes an elongate shaft 304 with a radially expandable balloon 306 disposed near a distal end of the elongate shaft 304. A stent 308 having a proximal portion 322, a distal portion 314 and a side hole 320 is disposed over the balloon 306. The distal portion 314 is crimped to the balloon 306 to prevent ejection during delivery, while the proximal portion 322 is partially crimped to the balloon 306 so the second catheter 330 may be slidably advanced under the proximal portion 322 of stent 308. The first catheter 302 is a rapid exchange catheter (RX) having a guidewire lumen 312 extending from the distal guidewire port 310 at the distal end of the elongate shaft 304 to a proximal guidewire port 311, which is closer to the distal guidewire port 310 than the proximal end of the catheter shaft 304. A connector 316 is coupled with the proximal end of the elongate shaft 304. The connector 316 is for example a Luer connector, and this allows easy coupling with an Indeflator or other device for inflation of the balloon 306. The first catheter 302 also includes a hollow exchange port tube 324 coupled to the elongate shaft 304. The hollow exchange port tube 324 may be coextruded with the first shaft 304, or it may be bonded or otherwise attached thereto using techniques known to those skilled in the art. The hollow exchange port tube 324 may alternatively be coupled with the other shaft 332. The hollow exchange port tube 324 includes a central channel 326 extending therethrough and is sized to slidably receive a portion of the second catheter 330. Radiopaque markers may be placed at different locations along the shaft 304, often near the balloon 306 and/or stent 308, to help mark the proximal and distal ends of the stent 308 or balloon 306, as well as to facilitate alignment of the two catheters during stent deployment, as discussed elsewhere in this specification.

The second catheter 330 includes an elongate shaft 332 with a radially expandable balloon 340 disposed near a distal end of the elongate shaft 332. A stent 342 is disposed over balloon 340. The stent 342 may have a length that matches the working length of the balloon 340, or the stent length may be shorter than the balloon working length. In some examples, the stent 342 is shorter than the working length of the balloon 340 so that a proximal portion of the balloon 340 is unconstrained by the stent 342, and this unconstrained portion of the balloon 340 may be slidably advanced or retracted through side hole 320 and under proximal portion 322 of stent 308 as will be discussed below. Stent 342 is crimped to balloon 340 to prevent ejection during delivery. At least a portion of balloon 340 and stent 342 are distally offset relative to balloon 306 and stent 308 so as to minimize profile of the device. In this example the distal stent 342 may be deployed in a main branch of the vessel and the other stent 308 may be deployed in a side branch of the vessel. Alternatively, the distal stent 342 may be deployed in a side branch of a vessel and the other stent 308 may be deployed in the main branch of a vessel.

The second catheter 330 is a rapid exchange catheter (RX) having a guidewire lumen 334 extending from the distal guidewire port 338 at the distal end of the elongate shaft 332 to a proximal guidewire port 336, which is closer to the distal port 338 than the proximal end of the catheter shaft 332. The proximal guidewire port 336 is also unobstructed by the hollow exchange port tube 324 and may be distal thereto. A connector 344, for example a Luer connector, is connected to the proximal end of the elongate shaft 332 and allows an Indeflator or other device to be coupled with an inflation lumen (not shown) in elongate shaft 332 for inflation of balloon 340. A portion of shaft 332 is disposed in the central channel 326 of the hollow exchange port tube 324, and this helps keep the two catheter shafts 304, 332 parallel and prevents tangling during delivery and as shaft 332 is slidably advanced or retracted relative to shaft 304. Also, another portion of shaft 332 is disposed under proximal portion 322 of stent 308. The second catheter 330 may also be slidably advanced or retracted under the proximal portion 322 of stent 308 so that the shaft 332 passes through the side hole 320 in stent 308. Radiopaque markers may be placed at different locations on the shaft 332, often near the balloon 340 or stent 342, to help mark the proximal and distal ends of the stent 342 or balloon 340, as well as to facilitate alignment of the two catheters during stent deployment, as discussed elsewhere in this specification.

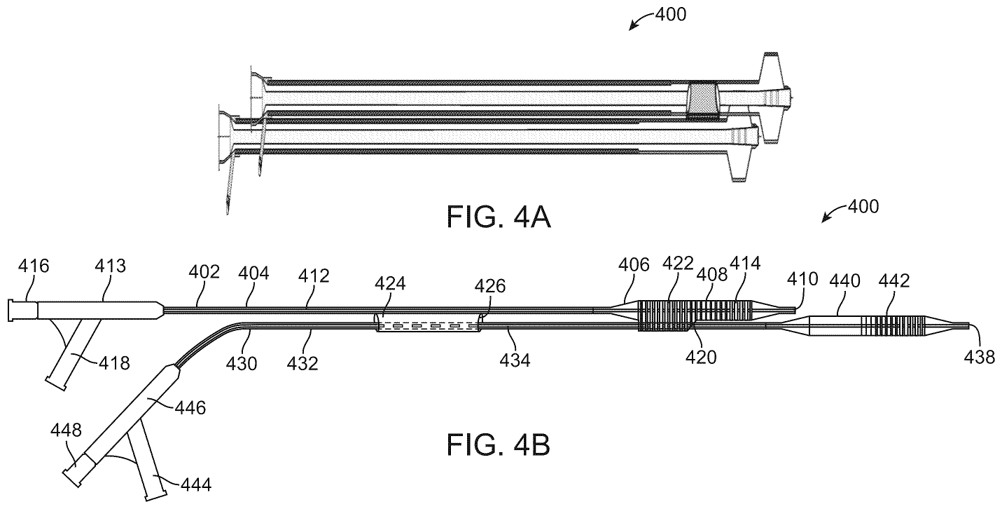

FIG. 4A illustrates a cross-sectional view of one example of a catheter system 400 with the mother and daughter catheters both having an over-the-wire design. In this particular example one of the catheters has a hollow exchange port embedded in its side and the other catheter does not have a hollow exchange port. The catheter without the exchange port is loaded onto the catheter with an exchange port. Typically, the catheter would have to be loaded prior to having a stent crimped over the balloon portion.

FIG. 4B more clearly illustrates the features of the catheter system 400 in FIG. 4A. The catheter system 400 includes a first catheter 402 and a second catheter 430. The first catheter 402 includes an elongate shaft 404 with a radially expandable balloon 406 disposed near a distal end of the elongate shaft 404. A stent 408 having a proximal portion 422, a distal portion 414 and a side hole 420 is disposed over the balloon 406. The distal portion 414 is crimped to the balloon 406 to prevent ejection during delivery, while the proximal portion 422 is partially crimped to the balloon 406 so the second catheter 430 may be slidably advanced under the proximal portion 422 of stent 408. The first catheter 402 is an over-the-wire (OTW) catheter having a guidewire lumen 412 extending from the distal guidewire port 410 at the distal end of the elongate shaft 404 to the proximal end of the elongate shaft 404 into Y-adapter 413 having a connector 416. The connector 416 is for example a Luer connector, and this allows easy coupling with a syringe or other device for lumen flushing or injecting contrast media. When unconnected, the guidewire lumen 412 exits via connector 416. A second connector 418, also for example a Luer connector, allows attachment of an Indeflator or other device to the catheter for inflation of the balloon 406 via an inflation lumen (not shown) in the elongate shaft 404. The first catheter 402 also includes a hollow exchange port tube 424 coupled to the elongate shaft 404. The hollow exchange port tube 424 may be coextruded with the first shaft 404, or it may be bonded or otherwise attached thereto using techniques known to those skilled in the art. The hollow exchange port tube 424 may alternatively be coupled with the other shaft 432. The hollow exchange port tube 424 includes a central channel 426 extending therethrough and is sized to slidably receive a portion of the second catheter 430. Radiopaque markers may be placed at different locations along the shaft 404, often near the balloon 406 and/or stent 408, to help mark the proximal and distal ends of the stent 408 or balloon 406, as well as to facilitate alignment of the two catheters during stent deployment, as discussed elsewhere in this specification.

The second catheter 430 includes an elongate shaft 432 with a radially expandable balloon 440 disposed near a distal end of the elongate shaft 432. A stent 442 is disposed over balloon 440. The stent 442 may have a length that matches the working length of the balloon 440, or the stent length may be shorter than the balloon working length. In some examples, the stent 442 is shorter than the working length of the balloon 440 so that a proximal portion of the balloon 440 is unconstrained by the stent 442 and this unconstrained portion of the balloon 440 may be slidably advanced or retracted through side hole 420 and under proximal portion 422 of stent 408 as will be discussed below. Stent 442 is crimped to balloon 440 to prevent ejection during delivery. At least a portion of balloon 440 and stent 442 are distally offset relative to balloon 406 and stent 408 so as to minimize profile of the device. In this example the distal stent 442 may be deployed in a main branch of the vessel and the other stent 408 may be deployed in a side branch of the vessel. Alternatively, the distal stent 442 may be deployed in a side branch of a vessel and the other stent 408 may be deployed in the main branch of a vessel.

The second catheter 430 is an over-the-wire (OTW) catheter having a guidewire lumen 434 extending from the distal guidewire port 438 at the distal end of the elongate shaft 432 to the proximal end of the elongate shaft 432 into Y-adapter 446 having a connector 448. The connector 448 is for example a Luer connector, and this allows easy coupling with a syringe or other device for lumen flushing or injecting contrast media. When unconnected, the guidewire lumen 434 exits via connector 448. A second connector 444, also for example a Luer connector, allows attachment of an Indeflator or other device to the catheter for inflation of the balloon 440 via an inflation lumen (not shown) in the elongate shaft 432. A portion of shaft 432 is disposed in the central channel 426 of the hollow exchange port tube 424 and this helps keep the two catheter shafts 404, 432 parallel and prevents tangling during delivery and as shaft 432 is slidably advanced or retracted relative to shaft 404. Also, another portion of shaft 432 is disposed under proximal portion 422 of stent 408. The second catheter 430 may also be slidably advanced or retracted under the proximal portion 422 of stent 408 so that the shaft 432 passes through the side hole 420 in stent 408. Radiopaque markers may be placed at different locations on the shaft 432, often near the balloon 440 or stent 442, to help mark the proximal and distal ends of the stent 442 or balloon 440, as well as to facilitate alignment of the two catheters during stent deployment, as discussed elsewhere in this specification.

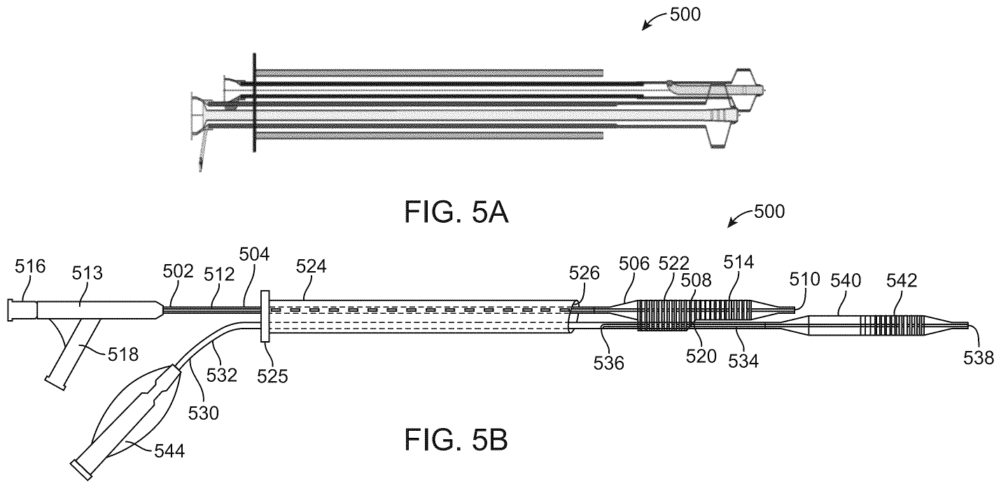

FIGS. 5A, 6A, 7A, and 8A illustrate an end-to-end capture tube that connects the catheters together. The capture tube keeps the catheters from tangling. The capture tube for example remains in place during the entire clinical procedure. In these examples, the capture tube is a thin polymer hollow straw that covers the mother and daughter catheters from a point about 10 centimeters distal to the Indeflator attachment to a distal point that is about 10 centimeters proximal from the rapid exchange catheter's proximal rapid exchange port.

FIG. 5A illustrates a catheter system 500 having a distal daughter catheter with a rapid exchange configuration and a proximal mother catheter with an over-the-wire configuration. FIG. 5B more clearly illustrates the features of the catheter system 500 seen in FIG. 5A. The catheter system 500 includes a first catheter 502 and a second catheter 530. The first catheter 502 includes an elongate shaft 504 with a radially expandable balloon 506 disposed near a distal end of the elongate shaft 504. A stent 508 having a proximal portion 522 a distal portion 514 and a side hole 520 is disposed over the balloon 506. The distal portion 514 is crimped to the balloon 506 to prevent ejection during delivery, while the proximal portion 522 is partially crimped to the balloon 506 so the second catheter 530 may be slidably advanced under the proximal portion 522 of stent 508. The first catheter 502 is an over-the-wire (OTW) catheter having a guidewire lumen 512 extending from the distal guidewire port 510 at the distal end of the elongate shaft 504 to the proximal end of the elongate shaft 504 into Y-adapter 513 having a connector 516. The connector 516 is for example a Luer connector, and this allows easy coupling with a syringe or other device for lumen flushing or injecting contrast media. When unconnected, the guidewire lumen 512 exits via connector 516. A second connector 518, also for example a Luer connector, allows attachment of an Indeflator or other device to the first catheter 502 for inflation of the balloon 506 via an inflation lumen (not shown) in the elongate shaft 504. The first catheter 502 is disposed in the central channel 526 of a capture tube 524. Central channel 526 is sized to fit both shafts 504, 532 and allow slidable movement thereof. Shaft 504 is slidable in the central channel 526, or it may be locked with a compression fitting such as a locking collar 525 such as a Tuohy-Borst compression fitting. Radiopaque markers may be placed at different locations along the shaft 504, often near the balloon 506 and/or stent 508, to help mark the proximal and distal ends of the stent or balloon, as well to facilitate alignment of the two catheters during stent deployment, as discussed elsewhere in this specification.

The second catheter 530 includes an elongate shaft 532 with a radially expandable balloon 540 disposed near a distal end of the elongate shaft 532. A stent 542 is disposed over balloon 540. The stent 542 may have a length that matches the working length of the balloon 540, or the stent length may be shorter than the balloon working length. In some examples, the stent 542 is shorter than the working length of the balloon 540 so that a proximal portion of the balloon 540 is unconstrained by the stent 542, and this unconstrained portion of the balloon 540 may be slidably advanced or retracted through side hole 520 and under proximal portion 522 of stent 508 as will be discussed below. Stent 542 is crimped to balloon 540 to prevent ejection during delivery. At least a portion of balloon 540 and stent 542 are distally offset relative to balloon 506 and stent 508 so as to minimize profile of the device. In this example the distal stent 542 may be deployed in a main branch of the vessel and the other stent 508 may be deployed in a side branch of the vessel. Alternatively, the distal stent 542 may be deployed in a side branch of a vessel and the other stent 508 may be deployed in the main branch of a vessel.

The second catheter 530 is a rapid exchange catheter (RX) having a guidewire lumen 534 extending from the distal guidewire port 538 at the distal end of the elongate shaft 532 to a proximal guidewire port 536, which is closer to the distal guidewire port 538 than the proximal end of the catheter shaft 532. The proximal guidewire port 536 is also unobstructed by the capture tube 524 and may be distal thereto. A connector 544, for example a Luer connector, is connected to the proximal end of the elongate shaft 532 and allows an Indeflator or other device to be coupled with an inflation lumen (not shown) in elongate shaft 532 for inflation of balloon 540. A portion of shaft 532 is disposed in the central channel 526 of the capture tube 524, and this helps keep the two catheter shafts 504, 532 parallel and prevents tangling during delivery and as shaft 532 is slidably advanced in the central channel 526. Locking collar 525 may be used to lock elongate shafts 504, 532 in the capture tube 524 to prevent axial movement. The compression fitting may be a Tuohy-Borst fitting. Also, another portion of shaft 532 is disposed under proximal portion 522 of stent 508. The second catheter 530 may also be slidably advanced or retracted under the proximal portion 522 of stent 508 so that the shaft 532 passes through the side hole 520 in stent 508. Radiopaque markers may be placed at different locations on the shaft 532, often near the balloon 540 or stent 542, to help mark the proximal and distal ends of the stent or balloon, as well as to facilitate alignment of the two catheters during stent deployment, as discussed elsewhere in this specification.

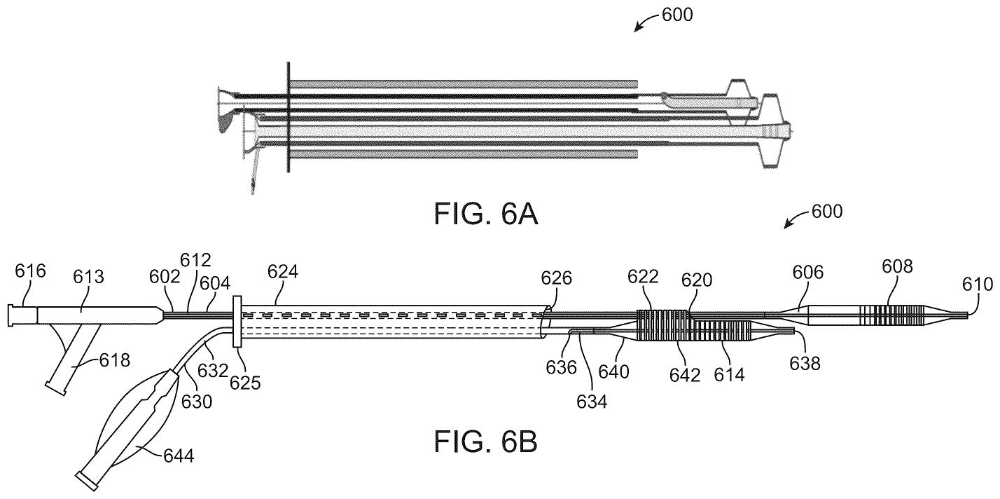

FIG. 6A illustrates a catheter system 600 having a distal daughter catheter with an over-the-wire design and a proximal mother catheter with a rapid exchange design. FIG. 6B more clearly illustrates the features of the catheter system 600 in FIG. 6A. The catheter system 600 includes a first catheter 602 and a second catheter 630. The first catheter 602 includes an elongate shaft 604 with a radially expandable balloon 606 disposed near a distal end of the elongate shaft 604, and a stent 608 disposed over the balloon 606. The stent 608 may be the same length as the working length of the balloon 606, or it may be shorter. In some examples, the stent 608 is shorter than the working length of balloon 606 such that a proximal portion of balloon 606 remains unconstrained by stent 608. The proximal portion of balloon 606 may be slidably advanced and retracted under stent 642 via side hole 620. Stent 608 is crimped to the balloon 606 to prevent ejection during delivery. The first catheter 602 is an over-the-wire (OTW) catheter having a guidewire lumen 612 extending from the distal guidewire port 610 at the distal end of the elongate shaft 604 to the proximal end of the elongate shaft 604 into Y-adapter 613 having a connector 616. The connector 616 is for example a Luer connector, and this allows easy coupling with a syringe or other device for lumen flushing or injecting contrast media. When unconnected, the guidewire lumen 612 exits via connector 616. A second connector 618, also for example a Luer connector, allows attachment of an Indeflator or other device to the first catheter 602 for inflation of the balloon 606 via an inflation lumen (not shown) in the elongate shaft 604. The first catheter 602 is disposed in the central channel 626 of a capture tube 624. Central channel 626 is sized to fit both shafts 604, 632 and allow slidable movement thereof. Shaft 604 is slidable in the central channel 626, or it may be locked with a locking collar 625 such as a Tuohy-Borst compression fitting. Radiopaque markers may be placed at different locations along the shaft 604, often near the balloon 606 and/or stent 608, to help mark the proximal and distal ends of the stent 608 or balloon 606, as well to facilitate alignment of the two catheters during stent deployment, as discussed elsewhere in this specification.

The second catheter 630 includes an elongate shaft 632 with a radially expandable balloon 640 disposed near a distal end of the elongate shaft 632. A stent 642 having a proximal portion 622, a distal portion 614, and a side hole 620 is disposed over balloon 640. The distal portion 614 is crimped to balloon 640 to prevent ejection during delivery, while the proximal portion 622 is partially crimped to balloon 640 so elongate shaft 604 may be slidably advanced or retracted under the proximal portion 622 of stent 642. The stent 642 may for example have a length that matches the working length of the balloon 640, or the stent length may be shorter than the balloon working length. At least a portion of balloon 606 and stent 608 are distally offset relative to balloon 640 and stent 642 so as to minimize the profile of the device. In this example the distal stent 608 may be deployed in a main branch of the vessel and the other stent 642 may be deployed in a side branch of the vessel. Alternatively, the distal stent 608 may be deployed in a side branch of a vessel and the other stent 642 may be deployed in the main branch of a vessel.

The second catheter 630 is a rapid exchange catheter (RX) having a guidewire lumen 634 extending from the distal guidewire port 638 at the distal end of the elongate shaft 632 to a proximal guidewire port 636, which is closer to the distal guidewire port 638 than the proximal end of the catheter shaft 632. The proximal guidewire port 636 is also unobstructed by the capture tube 624 and may be distal thereto. A connector 644, for example a Luer connector, is connected to the proximal end of the elongate shaft 632 and allows an Indeflator or other device to be coupled with an inflation lumen (not shown) in elongate shaft 632 for inflation of balloon 640. A portion of shaft 632 is disposed in the central channel 626 of the capture tube 624 and this helps keep the two catheter shafts 604, 632 parallel and prevents tangling during delivery and as shaft 604 is slidably advanced in the central channel 626. Locking collar 625 may be used to lock elongate shafts 604, 632 in the capture tube 624 to prevent axial movement. The compression fitting may be a Tuohy-Borst fitting. Also, a portion of shaft 604 is disposed under proximal portion 622 of stent 642. The first catheter 602 may be slidably advanced or retracted under the proximal portion 622 of stent 642 so that the shaft 604 passes through the side hole 620 in stent 642. Radiopaque markers may be placed at different locations on the shaft 632, often near the balloon 640 or stent 642, to help mark the proximal and distal ends of the stent or balloon, as well to facilitate alignment of the two catheters during stent deployment, as discussed elsewhere in this specification.

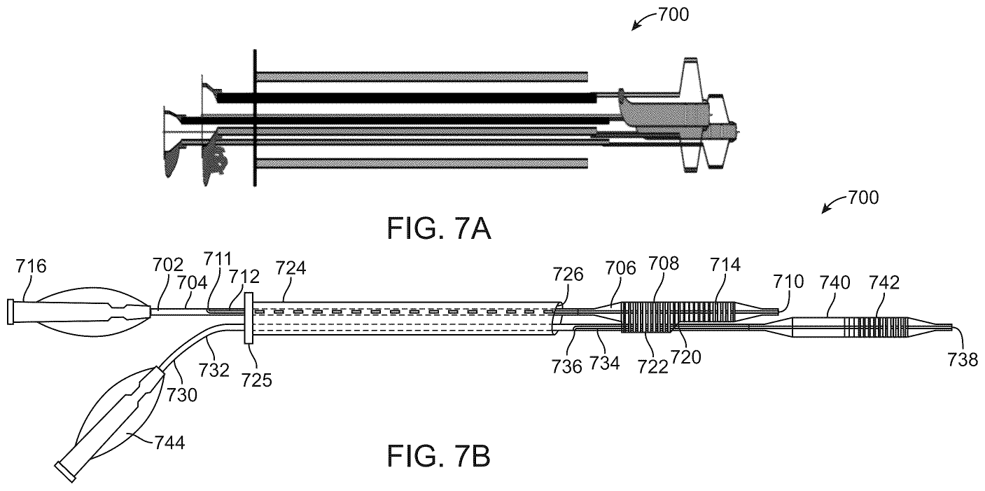

FIG. 7A shows a catheter system 700 having dual rapid exchange mother and daughter catheters so the end point of the capture tube is for example about 10 centimeters proximal from the rapid exchange port on the distal most catheter. FIG. 7B more clearly illustrates the features of the catheter system 700 in FIG. 7A. The catheter system 700 includes a first catheter 702 and a second catheter 730. The first catheter 702 includes an elongate shaft 704 with a radially expandable balloon 706 disposed near a distal end of the elongate shaft 704. A stent 708 having a proximal portion 722, a distal portion 714 and a side hole 720 is disposed over the balloon 706. The distal portion 714 is crimped to the balloon 706 to prevent ejection during delivery, while the proximal portion 722 is partially crimped to the balloon 706 so the second catheter 730 may be slidably advanced under the proximal portion 722 of stent 708. The first catheter 702 is a rapid exchange catheter (RX) having a guidewire lumen 712 extending from the distal guidewire port 710 at the distal end of the elongate shaft 704 to a proximal guidewire port 711, which is closer to the distal guidewire port 710 than the proximal end of the catheter shaft 704. A connector 716 is coupled with the proximal end of the elongate shaft 704. The connector 716 is for example a Luer connector, and this allows easy coupling with an Indeflator or other device for inflation of the balloon 706. The first catheter 702 is disposed in the central channel 726 of a capture tube 724. Central channel 726 is sized to fit both shafts 704, 732 and allow slidable movement thereof. Shaft 704 is slidable in the central channel 726, or it may be locked with a locking collar 725 such as a Tuohy-Borst compression fitting. Radiopaque markers may be placed at different locations along the shaft 704, often near the balloon 706 and/or stent 708, to help mark the proximal and distal ends of the stent 708 or balloon 706, as well as to facilitate alignment of the two catheters during stent deployment, as discussed elsewhere in this specification.

The second catheter 730 includes an elongate shaft 732 with a radially expandable balloon 740 disposed near a distal end of the elongate shaft 732. A stent 742 is disposed over balloon 740. The stent 742 may have a length that matches the working length of the balloon 740, or the stent length may be shorter than the balloon working length. In some examples, the stent 742 is shorter than the working length of the balloon 740 so that a proximal portion of the balloon 740 is unconstrained by the stent 742, and this unconstrained portion of the balloon 740 may be slidably advanced or retracted through side hole 720 and under proximal portion 722 of stent 708 as will be discussed below. Stent 742 is crimped to balloon 740 to prevent ejection during delivery. At least a portion of balloon 740 and stent 742 are distally offset relative to balloon 706 and stent 708 so as to minimize profile of the device. In this example the distal stent 742 may be deployed in a main branch of the vessel and the other stent 708 may be deployed in a side branch of the vessel. Alternatively, the distal stent 742 may be deployed in a side branch of a vessel and the other stent 708 may be deployed in the main branch of a vessel.

The second catheter 730 is a rapid exchange catheter (RX) having a guidewire lumen 734 extending from the distal guidewire port 738 at the distal end of the elongate shaft 732 to a proximal guidewire port 736, which is closer to the distal guidewire port 738 than the proximal end of the catheter shaft 732. The proximal guidewire port 736 is also unobstructed by the capture tube 724 and may be distal thereto. A connector 744, for example a Luer connector, is connected to the proximal end of the elongate shaft 732 and allows an Indeflator or other device to be coupled with an inflation lumen (not shown) in elongate shaft 732 for inflation of balloon 740. A portion of shaft 732 is disposed in the central channel 726 of the capture tube 724 and this helps keep the two catheter shafts 704, 732 parallel and prevents tangling during delivery and as shaft 732 is slidably advanced in the central channel 726. Locking collar 725 may be used to lock elongate shafts 704, 732 in the capture tube 724 to prevent axial movement. The compression fitting may be a Tuohy-Borst fitting. Also, another portion of shaft 732 is disposed under proximal portion 722 of stent 708. The second catheter 730 may also be slidably advanced or retracted under the proximal portion 722 of stent 708 so that the shaft 732 passes through the side hole 720 in stent 708. Radiopaque markers may be placed at different locations on the shaft 732, often near the balloon 740 or stent 742, to help mark the proximal and distal ends of the stent 742 or balloon 740, as well as to facilitate alignment of the two catheters during stent deployment, as discussed elsewhere in this specification.

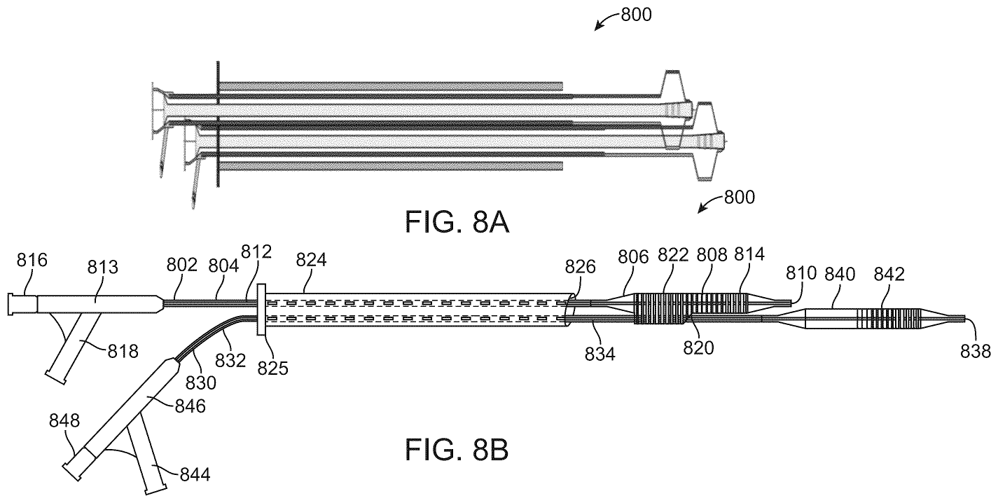

FIG. 8A embodies a catheter system 800 with dual over-the-wire designs; therefore, the capture tube ending point ends for example about 30 centimeters proximal from the balloon portion of the most distal catheter. FIG. 8B more clearly illustrates the features of the catheter system 800 in FIG. 8A. The catheter system 800 includes a first catheter 802 and a second catheter 830. The first catheter 802 includes an elongate shaft 804 with a radially expandable balloon 806 disposed near a distal end of the elongate shaft 804. A stent 808 having a proximal portion 822, a distal portion 814 and a side hole 820 is disposed over the balloon 806. The distal portion 814 is crimped to the balloon 806 to prevent ejection during delivery, while the proximal portion 822 is partially crimped to the balloon 806 so the second catheter 830 may be slidably advanced under the proximal portion 822 of stent 808. The first catheter 802 is an over-the-wire (OTW) catheter having a guidewire lumen 812 extending from the distal guidewire port 810 at the distal end of the elongate shaft 804 to the proximal end of the elongate shaft 804 into Y-adapter 813 having a connector 816. The connector 816 is for example a Luer connector, and this allows easy coupling with a syringe or other device for lumen flushing or injecting contrast media. When unconnected, the guidewire lumen 812 exits via connector 816. A second connector 818, also for example a Luer connector, allows attachment of an Indeflator or other device to the first catheter 802 for inflation of the balloon 806 via an inflation lumen (not shown) in the elongate shaft 804. The first catheter 802 is disposed in the central channel 826 of a capture tube 824. Central channel 826 is sized to fit both shafts 804, 832 and allow slidable movement thereof. Shaft 804 is slidable in the central channel 826, or it may be locked with a locking collar 825 such as a Tuohy-Borst compression fitting. Radiopaque markers may be placed at different locations along the shaft 804, often near the balloon 806 and/or stent 808, to help mark the proximal and distal ends of the stent 808 or balloon 806, as well to facilitate alignment of the two catheters during stent deployment, as discussed elsewhere in this specification.

The second catheter 830 includes an elongate shaft 832 with a radially expandable balloon 840 disposed near a distal end of the elongate shaft 832. A stent 842 is disposed over balloon 840. The stent 842 may have a length that matches the working length of the balloon 840, or the stent length may be shorter than the balloon working length. In some examples, the stent 842 is shorter than the working length of the balloon 840 so that a proximal portion of the balloon 840 is unconstrained by the stent 842, and this unconstrained portion of the balloon 840 may be slidably advanced or retracted through side hole 820 and under proximal portion 822 of stent 808 as will be discussed below. Stent 842 is crimped to balloon 840 to prevent ejection during delivery. At least a portion of balloon 840 and stent 842 are distally offset relative to balloon 806 and stent 808 so as to minimize profile of the device. In this example the distal stent 842 may be deployed in a main branch of the vessel and the other stent 808 may be deployed in a side branch of the vessel. Alternatively, the distal stent 842 may be deployed in a side branch of a vessel and the other stent 808 may be deployed in the main branch of a vessel.

The second catheter 830 is an over-the-wire (OTW) catheter having a guidewire lumen 834 extending from the distal guidewire port 838 at the distal end of the elongate shaft 832 to the proximal end of the elongate shaft 832 into Y-adapter 846 having a connector 848. The connector 848 is for example a Luer connector, and this allows easy coupling with a syringe or other device for lumen flushing or injecting contrast media. When unconnected, the guidewire lumen 834 exits via connector 848. A second connector 844, also for example a Luer connector, allows attachment of an Indeflator or other device to the catheter for inflation of the balloon 840 via an inflation lumen (not shown) in the elongate shaft 832. A portion of shaft 832 is disposed in the central channel 826 of the capture tube 824, and this helps keep the two catheter shafts 804, 832 parallel and prevents tangling during delivery and as shaft 832 is slidably advanced in the central channel 826. Locking collar 825 may be used to lock elongate shafts 804, 832 in the capture tube 824 to prevent axial movement. The compression fitting may be a Tuohy-Borst fitting. Also, another portion of shaft 832 is disposed under proximal portion 822 of stent 808. The second catheter 830 may also be slidably advanced or retracted under the proximal portion 822 of stent 808 so that the shaft 832 passes through the side hole 820 in stent 808. Radiopaque markers may be placed at different locations on the shaft 832, often near the balloon 840 or stent 842, to help mark the proximal and distal ends of the stent 842 or balloon 840, as well as to facilitate alignment of the two catheters during stent deployment, as discussed elsewhere in this specification.

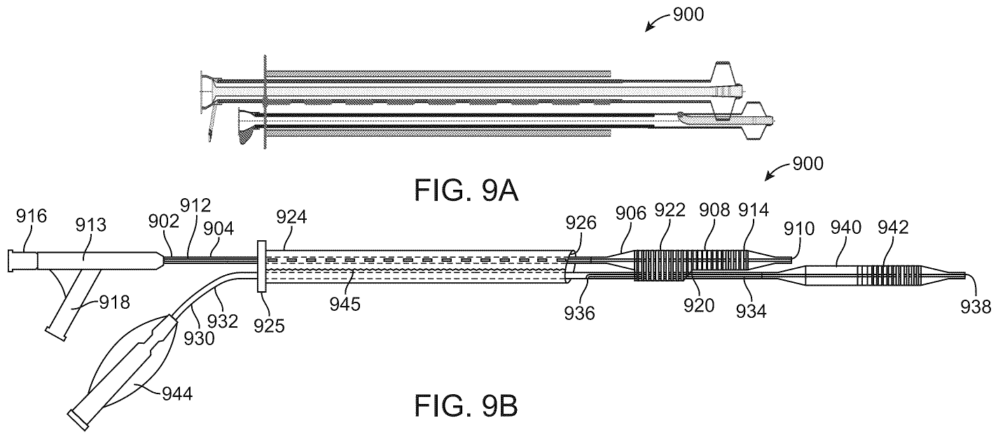

FIGS. 9A, 10A, 11A, and 12A illustrate a removable capture tube that is fitted over the dual catheters as described above, but the capture tube has a polymer appendage. Once the operator has the catheter system placed near the bifurcation, the operator can grab hold of the polymer appendage and pull the capture tube off of the catheters.

FIG. 9A illustrates a catheter system 900 having a distal daughter catheter with a rapid exchange configuration and a proximal mother catheter with an over-the-wire configuration. FIG. 9B more clearly illustrates the features of the catheter system 900 seen in FIG. 9A. The catheter system 900 includes a first catheter 902 and a second catheter 930. The first catheter 902 includes an elongate shaft 904 with a radially expandable balloon 906 disposed near a distal end of the elongate shaft 904. A stent 908 having a proximal portion 922, a distal portion 914 and a side hole 920 is disposed over the balloon 906. The distal portion 914 is crimped to the balloon 906 to prevent ejection during delivery, while the proximal portion 922 is partially crimped to the balloon 906 so the second catheter 930 may be slidably advanced under the proximal portion 922 of stent 908. The first catheter is an over-the-wire (OTW) catheter having a guidewire lumen 912 extending from the distal guidewire port 910 at the distal end of the elongate shaft 904 to the proximal end of the elongate shaft 904 into Y-adapter 913 having a connector 916. The connector 916 is for example a Luer connector, and this allows easy coupling with a syringe or other device for lumen flushing or injecting contrast media. When unconnected, the guidewire lumen 912 exits via connector 916. A second connector 918, also for example a Luer connector, allows attachment of an Indeflator or other device to the catheter for inflation of the balloon 906 via an inflation lumen (not shown) in the elongate shaft 904. The first catheter 902 is disposed in the central channel 926 of a capture tube 924 having a perforated region 945 along its longitudinal length. Central channel 926 is sized to fit both shafts 904, 932 and allow slidable movement thereof. Shaft 904 is slidable in the central channel 926, or it may be locked with a locking collar 925 such as a Tuohy-Borst compression fitting. Radiopaque markers may be placed at different locations along the shaft 904, often near the balloon 906 and/or stent 908, to help mark the proximal and distal ends of the stent 908 or balloon 906, as well to facilitate alignment of the two catheters during stent deployment, as discussed elsewhere in this specification. The perforated region 945 along the capture tube 924 allows the capture tube 924 to be easily peeled away from both catheter shafts 904, 932 once the catheters have been properly positioned and when no longer needed.

The second catheter 930 includes an elongate shaft 932 with a radially expandable balloon 940 disposed near a distal end of the elongate shaft 932. A stent 942 is disposed over balloon 940. The stent 942 may have a length that matches the working length of the balloon 940, or the stent length may be shorter than the balloon working length. In some examples, the stent 942 is shorter than the working length of the balloon 940 so that a proximal portion of the balloon 940 is unconstrained by the stent 942, and this unconstrained portion of the balloon 940 may be slidably advanced or retracted through side hole 920 and under proximal portion 922 of stent 908 as will be discussed below. Stent 942 is crimped to balloon 940 to prevent ejection during delivery. At least a portion of balloon 940 and stent 942 are distally offset relative to balloon 906 and stent 908 so as to minimize profile of the device. In this example the distal stent 942 may be deployed in a main branch of the vessel and the other stent 908 may be deployed in a side branch of the vessel. Alternatively, the distal stent 942 may be deployed in a side branch of a vessel and the other stent 908 may be deployed in the main branch of a vessel.

The second catheter 930 is a rapid exchange catheter (RX) having a guidewire lumen 934 extending from the distal guidewire port 938 at the distal end of the elongate shaft 932 to a proximal guidewire port 936, which is closer to the distal guidewire port 938 than the proximal end of the catheter shaft 932. The proximal guidewire port 936 is also unobstructed by the capture tube 924 and may be distal thereto. A connector 944, for example a Luer connector, is connected to the proximal end of the elongate shaft 932 and allows an Indeflator or other device to be coupled with an inflation lumen (not shown) in elongate shaft 932 for inflation of balloon 940. A portion of shaft 932 is disposed in the central channel 926 of the capture tube 924 and this helps keep the two catheter shafts 904, 932 parallel and prevents tangling during delivery and as shaft 932 is slidably advanced in the central channel 926. Locking collar 925 may be used to lock elongate shafts 904, 932 in the capture tube 924 to prevent axial movement. The compression fitting may be a Tuohy-Borst fitting. Also, another portion of shaft 932 is disposed under proximal portion 922 of stent 908. The second catheter 930 may also be slidably advanced or retracted under the proximal portion 922 of stent 908 so that the shaft 932 passes through the side hole 920 in stent 908. Capture tube 924 may be peeled away from shaft 932 by severing the perforated region 945. Radiopaque markers may be placed at different locations on the shaft 932, often near the balloon 940 or stent 942, to help mark the proximal and distal ends of the stent 942 or balloon 940, as well to facilitate alignment of the two catheters during stent deployment, as discussed elsewhere in this specification.

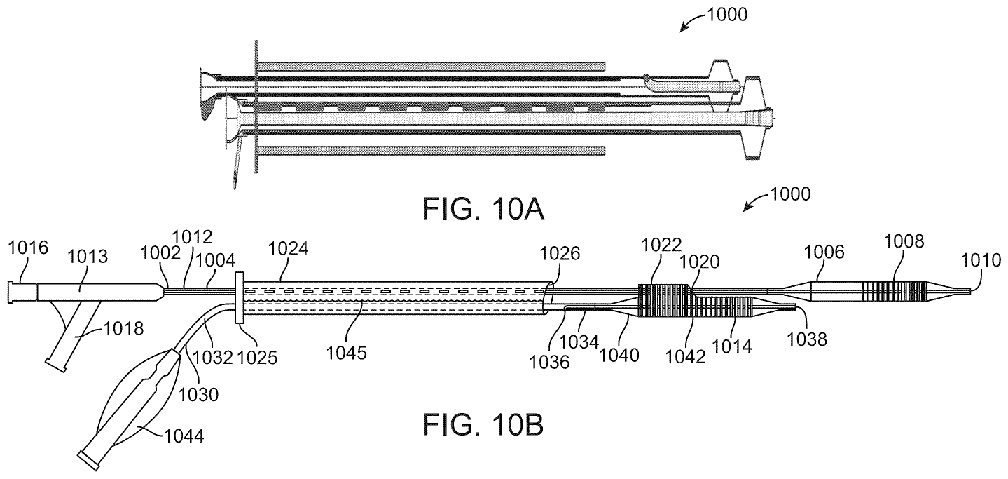

FIG. 10A illustrates a catheter system 1000 having a distal daughter catheter with an over-the-wire design and a proximal mother catheter with a rapid exchange design. FIG. 10B more clearly illustrates the features of the catheter system 1000 in FIG. 10A. The catheter system 1000 includes a first catheter 1002 and a second catheter 1030. The first catheter 1002 includes an elongate shaft 1004 with a radially expandable balloon 1006 disposed near a distal end of the elongate shaft 1004, and a stent 1008 disposed over the balloon 1006. The stent 1008 may be the same length as the working length of the balloon 1006, or it may be shorter. In some examples, the stent 1008 is shorter than the working length of balloon 1006 such that a proximal portion of balloon 1006 remains unconstrained by stent 1008. The proximal portion of balloon 1006 may be slidably advanced and retracted under stent 1042 via side hole 1020. Stent 1008 is crimped to the balloon 1006 to prevent ejection during delivery. The first catheter 1002 is an over-the-wire (OTW) catheter having a guidewire lumen 1012 extending from the distal guidewire port 1010 at the distal end of the elongate shaft 1004 to the proximal end of the elongate shaft 1004 into Y-adapter 1013 having a connector 1016. The connector 1016 is for example a Luer connector, and this allows easy coupling with a syringe or other device for lumen flushing or injecting contrast media. When unconnected, the guidewire lumen 1012 exits via connector 1016. A second connector 1018, also for example a Luer connector, allows attachment of an Indeflator or other device to the catheter for inflation of the balloon 1006 via an inflation lumen (not shown) in the elongate shaft 1004. The first catheter 1002 is disposed in the central channel 1026 of a capture tube 1024 having perforated region 1045. Central channel 1026 is sized to fit both shafts 1004, 1032 and allow slidable movement thereof. Shaft 1004 is slidable in the central channel 1026, or it may be locked with a locking collar 1025 such as a Tuohy-Borst compression fitting. Radiopaque markers may be placed at different locations along the shaft 1004, often near the balloon 1006 and/or stent 1008, to help mark the proximal and distal ends of the stent 1008 or balloon 1006, as well to facilitate alignment of the two catheters during stent deployment, as discussed elsewhere in this specification. The perforated region 1045 along the capture tube 1024 allows the capture tube 1024 to be easily peeled away from both catheter shafts 1004, 1032 once the catheters have been properly positioned and when no longer needed.

The second catheter 1030 includes an elongate shaft 1032 with a radially expandable balloon 1040 disposed near a distal end of the elongate shaft 1032. A stent 1042 having a proximal portion 1022, a distal portion 1014, and a side hole 1020 is disposed over balloon 1040. The distal portion 1014 is crimped to balloon 1040 to prevent ejection during delivery, while the proximal portion 1022 is partially crimped to balloon 1040 so elongate shaft 1004 may be slidably advanced or retracted under the proximal portion 1022 of stent 1042. The stent 1042 may for example have a length that matches the working length of the balloon 1040, or the stent length may be shorter than the balloon working length. At least a portion of balloon 1006 and stent 1008 are distally offset relative to balloon 1040 and stent 1042 so as to minimize profile of the device. In this example the distal stent 1008 may be deployed in a main branch of the vessel and the other stent 1042 may be deployed in a side branch of the vessel. Alternatively, the distal stent 1008 may be deployed in a side branch of a vessel and the other stent 1042 may be deployed in the main branch of a vessel.

The second catheter 1030 is a rapid exchange catheter (RX) having a guidewire lumen 1034 extending from the distal guidewire port 1038 at the distal end of the elongate shaft 1032 to a proximal guidewire port 1036, which is closer to the distal guidewire port 1038 than the proximal end of the catheter shaft 1032. The proximal guidewire port 1036 is also unobstructed by the capture tube 1024 and may be distal thereto. A connector 1044, for example a Luer connector, is connected to the proximal end of the elongate shaft 1032 and allows an Indeflator or other device to be coupled with an inflation lumen (not shown) in elongate shaft 1032 for inflation of balloon 1040. A portion of shaft 1032 is disposed in the central channel 1026 of the capture tube 1024 and this helps keep the two catheter shafts 1004, 1032 parallel and prevents tangling during delivery and as shaft 1032 is slidably advanced in the central channel 1026. Locking collar 1025 may be used to lock elongate shafts 1004, 1032 in the capture tube 1024 to prevent axial movement. The compression fitting may be a Tuohy-Borst fitting. Also, a portion of shaft 1004 is disposed under proximal portion 1022 of stent 1042. The first catheter 1002 may be slidably advanced or retracted under the proximal portion 1022 of stent 1042 so that the shaft 1004 passes through the side hole 1020 in stent 1042. Capture tube 1024 may be peeled away from shaft 1032 by severing the perforated region 1045. Radiopaque markers may be placed at different locations on the shaft 1032, often near the balloon 1040 or stent 1042, to help mark the proximal and distal ends of the stent 1042 or balloon 1040, as well as to facilitate alignment of the two catheters during stent deployment, as discussed elsewhere in this specification.

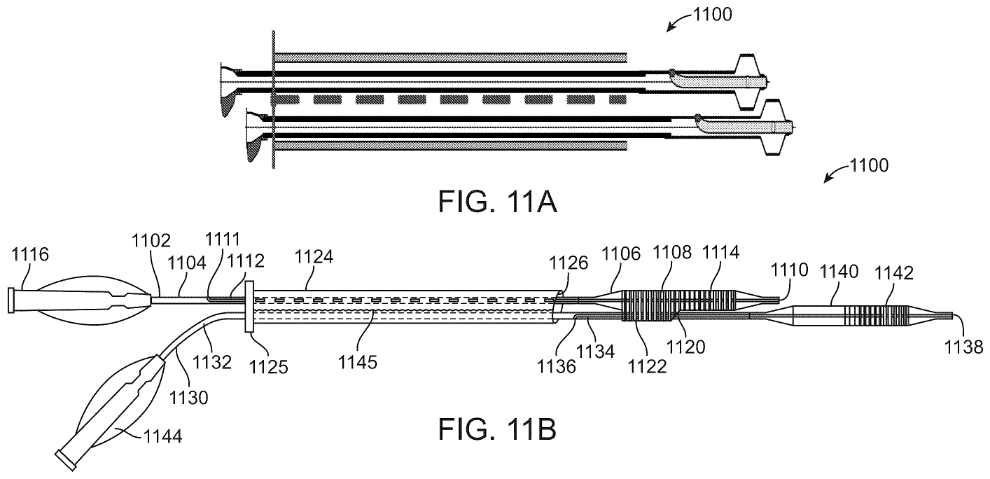

FIG. 11A illustrates a catheter system 1100 having dual rapid exchange design with a removable capture tube. FIG. 11B more clearly illustrates the features of the catheter system 1100 in FIG. 11A. The catheter system 1100 includes a first catheter 1102 and a second catheter 1130. The first catheter 1102 includes an elongate shaft 1104 with a radially expandable balloon 1106 disposed near a distal end of the elongate shaft 1104. A stent 1108 having a proximal portion 1122, a distal portion 1114 and a side hole 1120 is disposed over the balloon 1106. The distal portion 1114 is crimped to the balloon 1106 to prevent ejection during delivery, while the proximal portion 1122 is partially crimped to the balloon 1106 so the second catheter 1130 may be slidably advanced under the proximal portion 1122 of stent 1108. The first catheter 1102 is a rapid exchange catheter (RX) having a guidewire lumen 1112 extending from the distal guidewire port 1110 at the distal end of the elongate shaft 1104 to a proximal guidewire port 1111, which is closer to the distal guidewire port 1110 than the proximal end of the catheter shaft 1104. A connector 1116 is coupled with the proximal end of the elongate shaft 1104. The connector 1116 is for example a Luer connector, and this allows easy coupling with an Indeflator or other device for inflation of the balloon 1106. The first catheter 1102 is disposed in the central channel 1126 of a capture tube 1124 having a perforated region 1145. Central channel 1126 is sized to fit both shafts 1104, 1132 and allow slidable movement thereof. Shaft 1104 is slidable in the central channel 1126, or it may be locked with a locking collar 1125 such as a Tuohy-Borst compression fitting. Radiopaque markers may be placed at different locations along the shaft 1104, often near the balloon 1106 and/or stent 1108, to help mark the proximal and distal ends of the stent 1108 or balloon 1106, as well to facilitate alignment of the two catheters during stent deployment, as discussed elsewhere in this specification. The perforated region 1145 along the capture tube 1124 allows the capture tube 1124 to be easily peeled away from both catheter shafts 1104, 1132 once the catheters have been properly positioned and when no longer needed.

The second catheter 1130 includes an elongate shaft 1132 with a radially expandable balloon 1140 disposed near a distal end of the elongate shaft 1132. A stent 1142 is disposed over balloon 1140. The stent 1142 may have a length that matches the working length of the balloon 1140, or the stent length may be shorter than the balloon working length. In some examples, the stent 1142 is shorter than the working length of the balloon 1140 so that a proximal portion of the balloon 1140 is unconstrained by the stent 1142, and this unconstrained portion of the balloon 1140 may be slidably advanced or retracted through side hole 1120 and under proximal portion 1122 of stent 1108 as will be discussed below. Stent 1142 is crimped to balloon 1140 to prevent ejection during delivery. At least a portion of balloon 1140 and stent 1142 are distally offset relative to balloon 1106 and stent 1108 so as to minimize profile of the device. In this example the distal stent 1142 may be deployed in a main branch of the vessel and the other stent 1108 may be deployed in a side branch of the vessel. Alternatively, the distal stent 1142 may be deployed in a side branch of a vessel and the other stent 1108 may be deployed in the main branch of a vessel.

The second catheter 1130 is a rapid exchange catheter (RX) having a guidewire lumen 1134 extending from the distal guidewire port 1138 at the distal end of the elongate shaft 1132 to a proximal guidewire port 1136, which is closer to the distal guidewire port 1138 than the proximal end of the catheter shaft 1132. The proximal guidewire port 1136 is also unobstructed by the capture tube 1124 and may be distal thereto. A connector 1144, for example a Luer connector, is connected to the proximal end of the elongate shaft 1132 and allows an Indeflator or other device to be coupled with an inflation lumen (not shown) in elongate shaft 1132 for inflation of balloon 1140. A portion of shaft 1132 is disposed in the central channel 1126 of the capture tube 1124 and this helps keep the two catheter shafts 1104, 1132 parallel and prevents tangling during delivery and as shaft 1132 is slidably advanced in the central channel 1126. Locking collar 1125 may be used to lock elongate shafts 1104, 1132 in the capture tube 1124 to prevent axial movement. The compression fitting may be a Tuohy-Borst fitting. Also, another portion of shaft 1132 is disposed under proximal portion 1122 of stent 1108. The second catheter 1130 may also be slidably advanced or retracted under the proximal portion 1122 of stent 1108 so that the shaft 1132 passes through the side hole 1120 in stent 1108. Capture tube 1124 may be peeled away from shaft 1132 by severing the perforated region 1145. Radiopaque markers may be placed at different locations on the shaft 1132, often near the balloon 1140 or stent 1142, to help mark the proximal and distal ends of the stent 1142 or balloon 1140, as well to facilitate alignment of the two catheters during stent deployment, as discussed elsewhere in this specification.

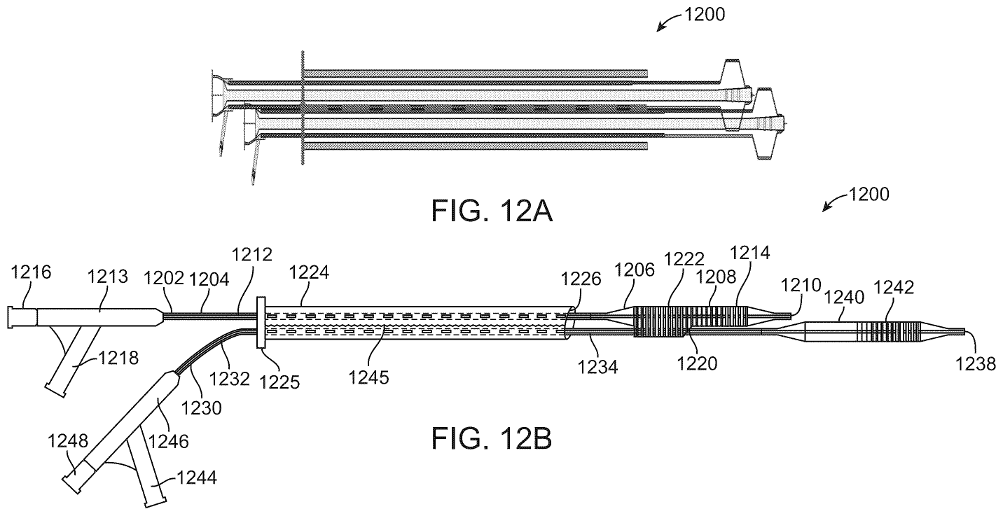

FIG. 12A illustrates a catheter system 1200 having dual over-the-wire design with a removable capture tube. FIG. 12B more clearly illustrates the features of the catheter system 1200 in FIG. 12A. The catheter system 1200 includes a first catheter 1202 and a second catheter 1230. The first catheter 1202 includes an elongate shaft 1204 with a radially expandable balloon 1206 disposed near a distal end of the elongate shaft 1204. A stent 1208 having a proximal portion 1222, a distal portion 1214 and a side hole 1220 is disposed over the balloon 1206. The distal portion 1214 is crimped to the balloon 1206 to prevent ejection during delivery, while the proximal portion 1222 is partially crimped to the balloon 1206 so the second catheter 1230 may be slidably advanced under the proximal portion 1222 of stent 1208. The first catheter 1202 is an over-the-wire (OTW) catheter having a guidewire lumen 1212 extending from the distal guidewire port 1210 at the distal end of the elongate shaft 1204 to the proximal end of the elongate shaft 1204 into Y-adapter 1213 having a connector 1216. The connector 1216 is for example a Luer connector, and this allows easy coupling with a syringe or other device for lumen flushing or injecting contrast media. When unconnected, the guidewire lumen 1212 exits via connector 1216. A second connector 1218, also for example a Luer connector, allows attachment of an Indeflator or other device to the first catheter 1202 for inflation of the balloon 1206 via an inflation lumen (not shown) in the elongate shaft 1204. The first catheter 1202 is disposed in the central channel 1226 of a capture tube 1224 having a perforated region 1245. Central channel 1226 is sized to fit both shafts 1204, 1232 and allow slidable movement thereof. Shaft 1204 is slidable in the central channel 1226, or it may be locked with a locking collar 1225 such as a Tuohy-Borst compression fitting. Radiopaque markers may be placed at different locations along the shaft 1204, often near the balloon 1206 and/or stent 1208, to help mark the proximal and distal ends of the stent 1208 or balloon 1206, as well to facilitate alignment of the two catheters during stent deployment, as discussed elsewhere in this specification. The perforated region 1245 along the capture tube 1224 allows the capture tube 1224 to be easily peeled away from both catheter shafts 1204, 1232 once the catheters have been properly positioned and when no longer needed.

The second catheter 1230 includes an elongate shaft 1232 with a radially expandable balloon 1240 disposed near a distal end of the elongate shaft 1232. A stent 1242 is disposed over balloon 1240. The stent 1242 may have a length that matches the working length of the balloon 1240, or the stent length may be shorter than the balloon working length. In some examples, the stent 1242 is shorter than the working length of the balloon 1240 so that a proximal portion of the balloon 1240 is unconstrained by the stent 1242, and this unconstrained portion of the balloon 1240 may be slidably advanced or retracted through side hole 1220 and under proximal portion 1222 of stent 1208 as will be discussed below. Stent 1242 is crimped to balloon 1240 to prevent ejection during delivery. At least a portion of balloon 1240 and stent 1242 are distally offset relative to balloon 1206 and stent 1208 so as to minimize profile of the device. In this example the distal stent 1242 may be deployed in a main branch of the vessel and the other stent 1208 may be deployed in a side branch of the vessel. Alternatively, the distal stent 1242 may be deployed in a side branch of a vessel and the other stent 1208 may be deployed in the main branch of a vessel.

The second catheter 1230 is an over-the-wire (OTW) catheter having a guidewire lumen 1234 extending from the distal guidewire port 1238 at the distal end of the elongate shaft 1232 to the proximal end of the elongate shaft 1232 into Y-adapter 1246 having a connector 1248. The connector 1248 is for example a Luer connector, and this allows easy coupling with a syringe or other device for lumen flushing or injecting contrast media. When unconnected, the guidewire lumen 1234 exits via connector 1248. A second connector 1244, also for example a Luer connector, allows attachment of an Indeflator or other device to the second catheter 1230 for inflation of the balloon 1240 via an inflation lumen (not shown) in the elongate shaft 1232. A portion of shaft 1232 is disposed in the central channel 1226 of the capture tube 1224 and this helps keep the two catheter shafts 1204, 1232 parallel and prevents tangling during delivery and as shaft 1232 is slidably advanced in the central channel 1226. Locking collar 1225 may be used to lock elongate shafts 1204, 1232 in the capture tube 1224 to prevent axial movement. The compression fitting may be a Tuohy-Borst fitting. Also, another portion of shaft 1232 is disposed under proximal portion 1222 of stent 1208. The second catheter 1230 may also be slidably advanced or retracted under the proximal portion 1222 of stent 1208 so that the shaft 1232 passes through the side hole 1220 in stent 1208. Capture tube 1224 may be peeled away from shaft 1232 by severing the perforated region 1245. Radiopaque markers may be placed at different locations on the shaft 1232, often near the balloon 1240 or stent 1242, to help mark the proximal and distal ends of the stent 1242 or balloon 1240, as well to facilitate alignment of the two catheters during stent deployment, as discussed elsewhere in this specification.

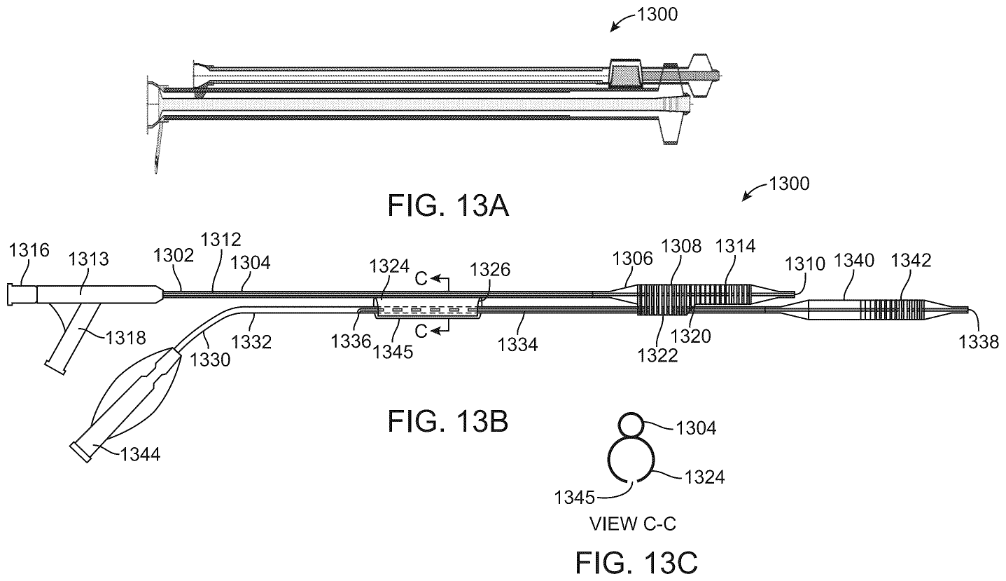

FIGS. 13A, 14A, 15A, and 16A illustrate a zipper that allows one catheter to snap into the other catheter. The zipper is essentially a groove that forms a concave receiving cross-section and is carved into a catheter's outer surface in a straight line. The groove can be a single groove over a certain portion of a catheter or it can run from end to end. Alternatively, the catheter can have a series of short grooves of 1 to 10 centimeters in length that run the length of the catheter or only a certain portion. Full length end-to-end zippers will have reduced profile and reduced friction with the vessel. The resulting groove can receive another catheter and prevent the catheters from dislodging while the operator is advancing the catheters to the bifurcation. Once at the site, the operator can still slidably move the catheters forward and back relative to each other. Mother catheters that utilize the groove can have fully crimped stents as described in several of the examples above; however, it is possible to allow operators to choose any commercially available catheter with or without a stent and mount the commercially available catheter via the zipper. The mother catheters with an empty zipper would have a mother stent fully crimped on the distal balloon portion. After loading the commercially available catheter, the operator would have to crimp the proximal portion of the mother stent in situ prior to beginning the clinical procedure. This option may be extremely valuable to operators who can reduce their total inventory of catheters but have more options for treating bifurcated lesions.

FIG. 13A illustrates a catheter system 1300 having a distal daughter catheter with an over-the-wire design and a proximal mother catheter with a rapid exchange design and a short zipper. FIG. 13B more clearly illustrates the features of the catheter system 1300 in FIG. 13A. The catheter system 1300 includes a first catheter 1302 and a second catheter 1330. The first catheter 1302 includes an elongate shaft 1304 with a radially expandable balloon 1306 disposed near a distal end of the elongate shaft 1304. A stent 1308 having a proximal portion 1322, a distal portion 1314 and a side hole 1320 is disposed over the balloon 1306. The distal portion 1314 is crimped to the balloon 1306 to prevent ejection during delivery, while the proximal portion 1322 is partially crimped to the balloon 1306 so the second catheter 1330 may be slidably advanced under the proximal portion 1322 of stent 1308. The first catheter is an over-the-wire (OTW) catheter having a guidewire lumen 1312 extending from the distal guidewire port 1310 at the distal end of the elongate shaft 1304 to the proximal end of the elongate shaft 1304 into Y-adapter 1313 having a connector 1316. The connector 1316 is for example a Luer connector, and this allows easy coupling with a syringe or other device for lumen flushing or injecting contrast media. When unconnected, the guidewire lumen 1312 exits via connector 1316. A second connector 1318, also for example a Luer connector, allows attachment of an Indeflator or other device to the first catheter 1302 for inflation of the balloon 1306 via an inflation lumen (not shown) in the elongate shaft 1304. The first catheter 1302 also includes a zipper or snap fitting 1324 coupled to the elongate shaft 1304. The snap fitting 1324 may be coextruded with the first shaft 1304, or it may be bonded or otherwise attached thereto using techniques known to those skilled in the art. The snap fitting 1324 may alternatively be coupled with the other shaft 1332. The snap fitting 1324 includes a central channel 1326 extending therethrough and is sized to slidably receive a portion of the second catheter 1330. An elongate slot 1345 extends along the entire length of the snap fitting 1324 and is sized so that shaft 1332 may snapped into the central channel 1326. FIG. 13C illustrates a partial cross-section of FIG. 13B taken along the line C-C and shows shaft 1304 with the snap fitting 1324. Radiopaque markers may be placed at different locations along the shaft 1304, often near the balloon 1306 and/or stent 1308, to help mark the proximal and distal ends of the stent 1308 or balloon 1306, as well as to facilitate alignment of the two catheters during stent deployment, as discussed elsewhere in this specification.

The second catheter 1330 includes an elongate shaft 1332 with a radially expandable balloon 1340 disposed near a distal end of the elongate shaft 1332. A stent 1342 is disposed over balloon 1340. The stent 1342 may have a length that matches the working length of the balloon 1340, or the stent length may be shorter than the balloon working length. In some examples, the stent 1342 is shorter than the working length of the balloon 1340 so that a proximal portion of the balloon 1340 is unconstrained by the stent 1342, and this unconstrained portion of the balloon 1340 may be slidably advanced or retracted through side hole 1320 and under proximal portion 1322 of stent 1308 as will be discussed below. Stent 1342 is crimped to balloon 1340 to prevent ejection during delivery. At least a portion of balloon 1340 and stent 1342 are distally offset relative to balloon 1306 and stent 1308 so as to minimize profile of the device. In this example the distal stent 1342 may be deployed in a main branch of the vessel and the other stent 1308 may be deployed in a side branch of the vessel. Alternatively, the distal stent 1342 may be deployed in a side branch of a vessel and the other stent 1308 may be deployed in the main branch of a vessel.

The second catheter 1330 is a rapid exchange catheter (RX) having a guidewire lumen 1334 extending from the distal guidewire port 1338 at the distal end of the elongate shaft 1332 to a proximal guidewire port 1336, which is closer to the distal guidewire port 1338 than the proximal end of the catheter shaft 1332. The proximal guidewire port 1336 is also unobstructed by the snap fitting 1324 and for example proximal thereto. A connector 1344, for example a Luer connector, is connected to the proximal end of the elongate shaft 1332 and allows an Indeflator or other device to be coupled with an inflation lumen (not shown) in elongate shaft 1332 for inflation of balloon 1340. A portion of shaft 1332 is snapped into the central channel 1326 of the snap fitting 1324 via slot 1345, and thus shaft 1332 may slide in channel 1326. This helps keep the two catheter shafts 1304, 1332 parallel and prevents tangling during delivery and as shaft 1332 is slidably advanced or retracted relative to shaft 1304. Also, another portion of shaft 1332 is disposed under proximal portion 1322 of stent 1308. The second catheter 1330 may also be slidably advanced or retracted under the proximal portion 1322 of stent 1308 so that the shaft 1332 passes through the side hole 1320 in stent 1308. Radiopaque markers may be placed at different locations on the shaft 1332, often near the balloon 1340 or stent 1342, to help mark the proximal and distal ends of the stent 1342 or balloon 1340, as well as to facilitate alignment of the two catheters during stent deployment, as discussed elsewhere in this specification.

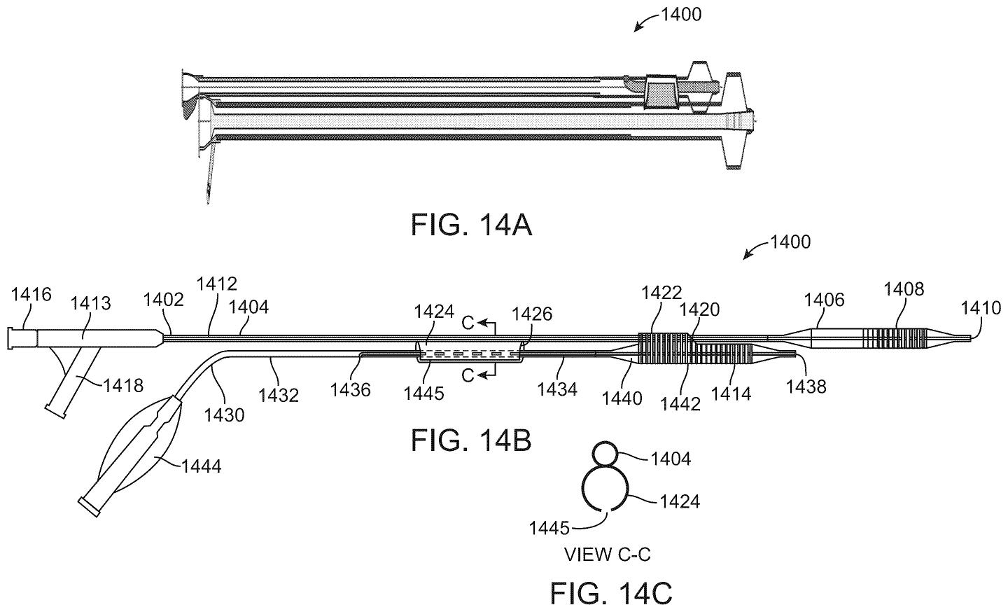

FIG. 14A illustrates a catheter system 1400 having a proximal mother catheter with a rapid exchange configuration and a distal daughter catheter having an over-the-wire configuration and a short zipper or snap fitting. FIG. 14B more clearly illustrates the features of the catheter system 1400 in FIG. 14A. The catheter system 1400 includes a first catheter 1402 and a second catheter 1430. The first catheter 1402 includes an elongate shaft 1404 with a radially expandable balloon 1406 disposed near a distal end of the elongate shaft 1404, and a stent 1408 disposed over the balloon 1406. The stent 1408 may be the same length as the working length of the balloon 1406, or it may be shorter. In some examples, the stent 1408 is shorter than the working length of balloon 1406 such that a proximal portion of balloon 1406 remains unconstrained by stent 1408. The proximal portion of balloon 1406 may be slidably advanced and retracted under stent 1442 via side hole 1420. Stent 1408 is crimped to the balloon 1406 to prevent ejection during delivery. The first catheter 1402 is an over-the-wire (OTW) catheter having a guidewire lumen 1412 extending from the distal guidewire port 1410 at the distal end of the elongate shaft 1404 to the proximal end of the elongate shaft 1404 into Y-adapter 1413 having a connector 1416. The connector 1416 is for example a Luer connector, and this allows easy coupling with a syringe or other device for lumen flushing or injecting contrast media. When unconnected, the guidewire lumen 1412 exits via connector 1416. A second connector 1418, also for example a Luer connector, allows attachment of an Indeflator or other device to the first catheter 1402 for inflation of the balloon 1406 via an inflation lumen (not shown) in the elongate shaft 1404. The first catheter 1402 also includes a zipper or snap fitting 1424 coupled to the elongate shaft 1404. The snap fitting 1424 may be coextruded with the first shaft 1404, or it may be bonded or otherwise attached thereto using techniques known to those skilled in the art. The snap fitting 1424 may alternatively be coupled with the other shaft 1432. The snap fitting 1424 includes a central channel 1426 extending therethrough and is sized to slidably receive a portion of the second catheter 1430. An elongate slot 1445 extends along the entire length of the snap fitting 1424 and is sized so that shaft 1432 may be snapped into the central channel 1426. FIG. 14C illustrates a partial cross-section of FIG. 14B taken along the line C-C and shows shaft 1404 with the snap fitting 1424. Radiopaque markers may be placed at different locations along the shaft 1404, often near the balloon 1406 and/or stent 1408, to help mark the proximal and distal ends of the stent 1408 or balloon 1406, as well as to facilitate alignment of the two catheters during stent deployment, as discussed elsewhere in this specification.

The second catheter 1430 includes an elongate shaft 1432 with a radially expandable balloon 1440 disposed near a distal end of the elongate shaft 1432. A stent 1442 having a proximal portion 1422, a distal portion 1414, and a side hole 1420 is disposed over balloon 1440. The distal portion 1414 is crimped to balloon 1440 to prevent ejection during delivery, while the proximal portion 1422 is partially crimped to balloon 1440 so elongate shaft 1404 may be slidably advanced or retracted under the proximal portion 1422 of stent 1442. The stent 1442 may for example have a length that matches the working length of the balloon 1440, or the stent length may be shorter than the balloon working length. At least a portion of balloon 1406 and stent 1408 are distally offset relative to balloon 1440 and stent 1442 so as to minimize profile of the device. In this example the distal stent 1408 may be deployed in a main branch of the vessel and the other stent 1442 may be deployed in a side branch of the vessel. Alternatively, the distal stent 1408 may be deployed in a side branch of a vessel and the other stent 1442 may be deployed in the main branch of a vessel.

The second catheter 1430 is a rapid exchange catheter (RX) having a guidewire lumen 1434 extending from the distal guidewire port 1438 at the distal end of the elongate shaft 1432 to a proximal guidewire port 1436, which is closer to the distal guidewire port 1438 than the proximal end of the catheter shaft 1432. The proximal guidewire port 1436 is also unobstructed by the snap fitting 1424 and for example proximal thereto. A connector 1444, for example a Luer connector, is connected to the proximal end of the elongate shaft 1432 and allows an Indeflator or other device to be coupled with an inflation lumen (not shown) in elongate shaft 1432 for inflation of balloon 1440. A portion of shaft 1432 is snapped into the central channel 1426 of the snap fitting 1424 via slot 1445, and thus shaft 1432 may slide in channel 1426. This helps keep the two catheter shafts 1404, 1432 parallel and prevents tangling during delivery and as shaft 1432 is slidably advanced or retracted relative to shaft 1404. Also, a portion of shaft 1404 is disposed under proximal portion 1422 of stent 1442. The first catheter 1402 may be slidably advanced or retracted under the proximal portion 1422 of stent 1442 so that the shaft 1404 passes through the side hole 1420 in stent 1442. Radiopaque markers may be placed at different locations on the shaft 1432, often near the balloon 1440 or stent 1442, to help mark the proximal and distal ends of the stent 1442 or balloon 1440, as well to facilitate alignment of the two catheters during stent deployment, as discussed elsewhere in this specification.

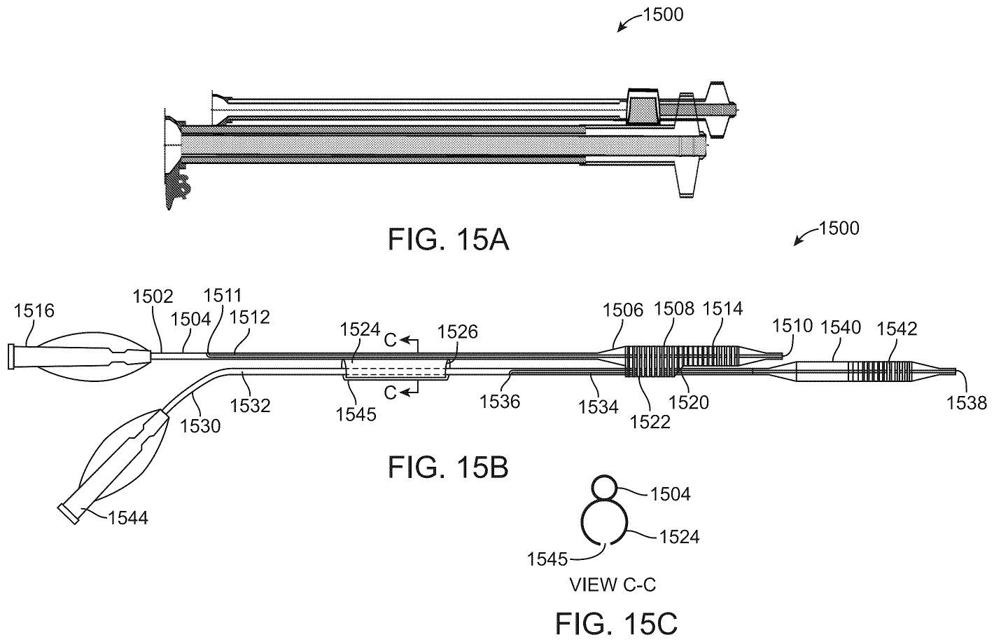

FIG. 15A illustrates a catheter system 1500 having dual rapid exchange design with a short zipper or snap fitting. FIG. 15B more clearly illustrates the features of the catheter system 1500 in FIG. 15A. The catheter system 1500 includes a first catheter 1502 and a second catheter 1530. The first catheter 1502 includes an elongate shaft 1504 with a radially expandable balloon 1506 disposed near a distal end of the elongate shaft 1504. A stent 1508 having a proximal portion 1522, a distal portion 1514 and a side hole 1520 is disposed over the balloon 1506. The distal portion 1514 is crimped to the balloon 1506 to prevent ejection during delivery, while the proximal portion 1522 is partially crimped to the balloon 1506 so the second catheter 1530 may be slidably advanced under the proximal portion 1522 of stent 1508. The first catheter 1502 is a rapid exchange catheter (RX) having a guidewire lumen 1512 extending from the distal guidewire port 1510 at the distal end of the elongate shaft 1504 to a proximal guidewire port 1511, which is closer to the distal guidewire port 1510 than the proximal end of the catheter shaft 1504. A connector 1516 is coupled with the proximal end of the elongate shaft 1504. The connector 1516 is for example a Luer connector, and this allows easy coupling with an Indeflator or other device for inflation of the balloon 1506. The first catheter 1502 also includes a zipper or snap fitting 1524 coupled to the elongate shaft 1504. The snap fitting 1524 may be coextruded with the first shaft 1504, or it may be bonded or otherwise attached thereto using techniques known to those skilled in the art. The snap fitting 1524 may alternatively be coupled with the other shaft 1532. The snap fitting 1524 includes a central channel 1526 extending therethrough and is sized to slidably receive a portion of the second catheter 1530. An elongate slot 1545 extends along the entire length of the snap fitting 1524 and is sized so that shaft 1532 may snapped into the central channel 1526. FIG. 15C illustrates a partial cross-section of FIG. 15B taken along the line C-C and shows shaft 1504 with the snap fitting 1524. Radiopaque markers may be placed at different locations along the shaft 1504, often near the balloon 1506 and/or stent 1508, to help mark the proximal and distal ends of the stent 1508 or balloon 1506, as well as to facilitate alignment of the two catheters during stent deployment, as discussed elsewhere in this specification.

The second catheter 1530 includes an elongate shaft 1532 with a radially expandable balloon 1540 disposed near a distal end of the elongate shaft 1532. A stent 1542 is disposed over balloon 1540. The stent 1542 may have a length that matches the working length of the balloon 1540, or the stent length may be shorter than the balloon working length. In some examples, the stent 1542 is shorter than the working length of the balloon 1540 so that a proximal portion of the balloon 1540 is unconstrained by the stent 1542, and this unconstrained portion of the balloon 1540 may be slidably advanced or retracted through side hole 1520 and under proximal portion 1522 of stent 1508 as will be discussed below. Stent 1542 is crimped to balloon 1540 to prevent ejection during delivery. At least a portion of balloon 1540 and stent 1542 are distally offset relative to balloon 1506 and stent 1508 so as to minimize profile of the device. In this example the distal stent 1542 may be deployed in a main branch of the vessel and the other stent 1508 may be deployed in a side branch of the vessel. Alternatively, the distal stent 1542 may be deployed in a side branch of a vessel and the other stent 1508 may be deployed in the main branch of a vessel.

The second catheter 1530 is a rapid exchange catheter (RX) having a guidewire lumen 1534 extending from the distal guidewire port 1538 at the distal end of the elongate shaft 1532 to a proximal guidewire port 1536, which is closer to the distal guidewire port 1538 than the proximal end of the catheter shaft 1532. The proximal guidewire port 1536 is also unobstructed by the snap fitting 1524 and may be distal thereto. A connector 1544, for example a Luer connector, is connected to the proximal end of the elongate shaft 1532 and allows an Indeflator or other device to be coupled with an inflation lumen (not shown) in elongate shaft 1532 for inflation of balloon 1540. A portion of shaft 1532 is snapped into the central channel 1526 of the snap fitting 1524 via slot 1545, and thus shaft 1532 may slide in channel 1526. This helps keep the two catheter shafts 1504, 1532 parallel and prevents tangling during delivery and as shaft 1532 is slidably advanced or retracted relative to shaft 1504. Also, another portion of shaft 1532 is disposed under proximal portion 1522 of stent 1508. The second catheter 1530 may also be slidably advanced or retracted under the proximal portion 1522 of stent 1508 so that the shaft 1532 passes through the side hole 1520 in stent 1508. Radiopaque markers may be placed at different locations on the shaft 1532, often near the balloon 1540 or stent 1542, to help mark the proximal and distal ends of the stent 1542 or balloon 1540, as well as to facilitate alignment of the two catheters during stent deployment, as discussed elsewhere in this specification.

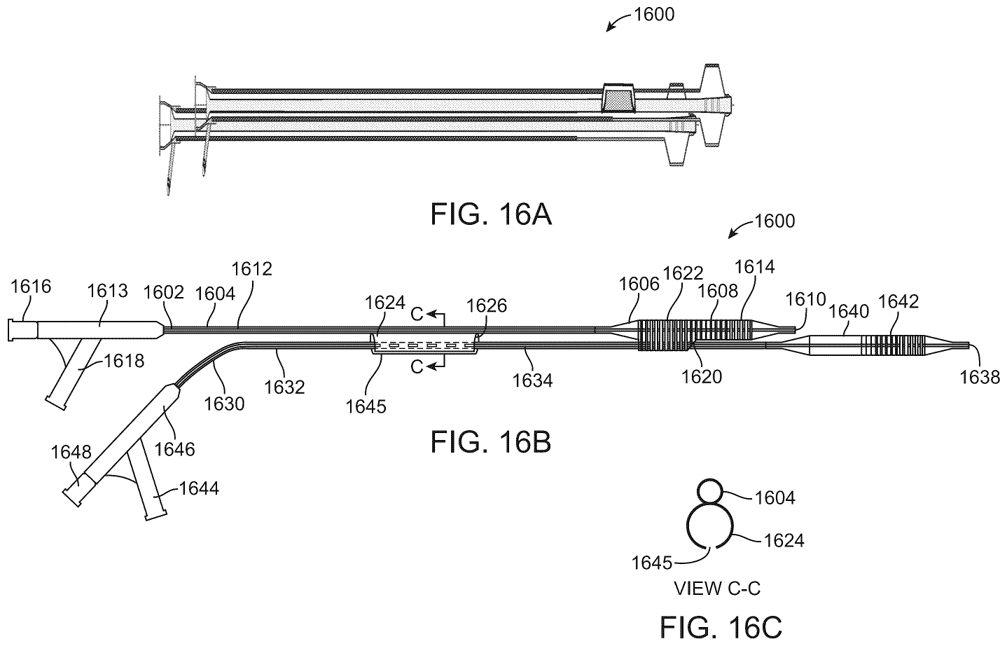

FIG. 16A illustrates a catheter system 1600 having a dual over-the-wire design with a short zipper or snap fitting. FIG. 16B more clearly illustrates the features of the catheter system 1600 in FIG. 16A. The catheter system 1600 includes a first catheter 1602 and a second catheter 1630. The first catheter 1602 includes an elongate shaft 1604 with a radially expandable balloon 1606 disposed near a distal end of the elongate shaft 1604. A stent 1608 having a proximal portion 1622, a distal portion 1614 and a side hole 1620 is disposed over the balloon 1606. The distal portion 1614 is crimped to the balloon 1606 to prevent ejection during delivery, while the proximal portion 1622 is partially crimped to the balloon 1606 so the second catheter 1630 may be slidably advanced under the proximal portion 1622 of stent 1608. The first catheter 1602 is an over-the-wire (OTW) catheter having a guidewire lumen 1612 extending from the distal guidewire port 1610 at the distal end of the elongate shaft 1604 to the proximal end of the elongate shaft 1604 into Y-adapter 1613 having a connector 1616. The connector 1616 is for example a Luer connector, and this allows easy coupling with a syringe or other device for lumen flushing or injecting contrast media. When unconnected, the guidewire lumen 1612 exits via connector 1616. A second connector 1618, also for example a Luer connector, allows attachment of an Indeflator or other device to the first catheter 1602 for inflation of the balloon 1606 via an inflation lumen (not shown) in the elongate shaft 1604. The first catheter 1602 also includes a zipper or snap fitting 1624 coupled to the elongate shaft 1604. The snap fitting 1624 may be coextruded with the first shaft 1604, or it may be bonded or otherwise attached thereto using techniques known to those skilled in the art. The snap fitting 1624 may alternatively be coupled with the other shaft 1632. The snap fitting 1624 includes a central channel 1626 extending therethrough and is sized to slidably receive a portion of the second catheter 1630. An elongate slot 1645 extends along the entire length of the snap fitting 1624 and is sized so that shaft 1636 may snapped into the central channel 1626. FIG. 16C illustrates a partial cross-section of FIG. 16B taken along the line C-C and shows shaft 1604 with the snap fitting 1624. Radiopaque markers may be placed at different locations along the shaft 1604, often near the balloon 1606 and/or stent 1608, to help mark the proximal and distal ends of the stent or balloon, as well as to facilitate alignment of the two catheters during stent deployment, as discussed elsewhere in this specification.

The second catheter 1630 includes an elongate shaft 1632 with a radially expandable balloon 1640 disposed near a distal end of the elongate shaft 1632. A stent 1642 is disposed over balloon 1640. The stent 1642 may have a length that matches the working length of the balloon 1640, or the stent length may be shorter than the balloon working length. In some examples, the stent 1642 is shorter than the working length of the balloon 1640 so that a proximal portion of the balloon 1640 is unconstrained by the stent 1642, and this unconstrained portion of the balloon 1640 may be slidably advanced or retracted through side hole 1620 and under proximal portion 1622 of stent 1608 as will be discussed below. Stent 1642 is crimped to balloon 1640 to prevent ejection during delivery. At least a portion of balloon 1640 and stent 1642 are distally offset relative to balloon 1606 and stent 1608 so as to minimize profile of the device. In this example the distal stent 1642 may be deployed in a main branch of the vessel and the other stent 1608 may be deployed in a side branch of the vessel. Alternatively, the distal stent 1642 may be deployed in a side branch of a vessel and the other stent 1608 may be deployed in the main branch of a vessel.

The second catheter 1630 is an over-the-wire (OTW) catheter having a guidewire lumen 1634 extending from the distal guidewire port 1638 at the distal end of the elongate shaft 1632 to the proximal end of the elongate shaft 1632 into Y-adapter 1646 having a connector 1648. The connector 1648 is for example a Luer connector, and this allows easy coupling with a syringe or other device for lumen flushing or injecting contrast media. When unconnected, the guidewire lumen 1634 exits via connector 1648. A second connector 1644, also for example a Luer connector, allows attachment of an Indeflator or other device to the second catheter 1630 for inflation of the balloon 1640 via an inflation lumen (not shown) in the elongate shaft 1632. A portion of shaft 1632 is snapped into the central channel 1626 of the snap fitting 1624 via slot 1645, and thus shaft 1632 may slide in channel 1626. This helps keep the two catheter shafts 1604, 1632 parallel and prevents tangling during delivery and as shaft 1632 is slidably advanced or retracted relative to shaft 1604. Also, another portion of shaft 1632 is disposed under proximal portion 1622 of stent 1608. The second catheter 1630 may also be slidably advanced or retracted under the proximal portion 1622 of stent 1608 so that the shaft 1632 passes through the side hole 1620 in stent 1608. Radiopaque markers may be placed at different locations on the shaft 1632, often near the balloon 1640 or stent 1642, to help mark the proximal and distal ends of the stent 1642 or balloon 1640, as well to facilitate alignment of the two catheters during stent deployment, as discussed elsewhere in this specification.

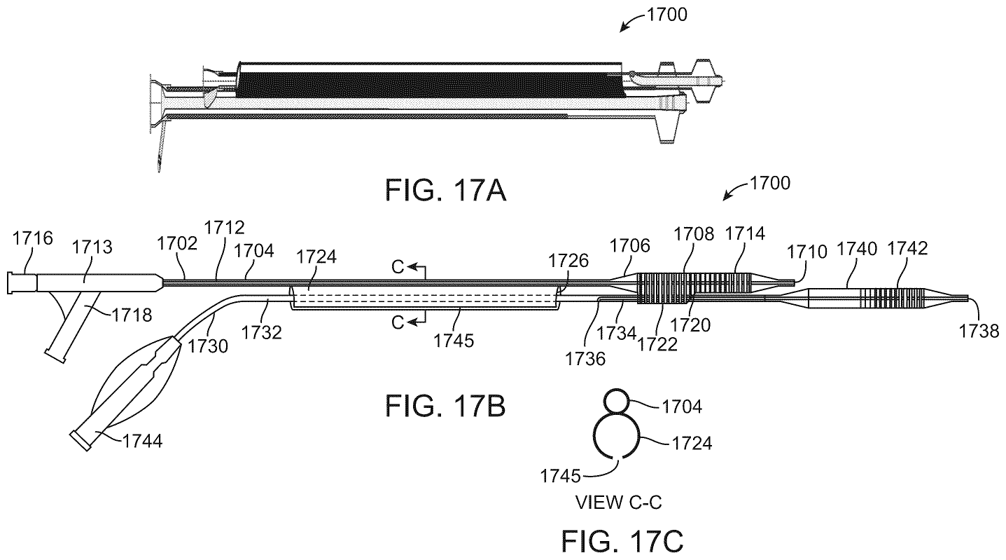

FIG. 17A illustrates a catheter system 1700 having a distal daughter catheter with a rapid-exchange configuration and a proximal mother catheter with an over-the-wire configuration and an end-to-end zipper, or snap fitting. This example is similar to that shown in FIGS. 13A-13B, with the major difference being the length of the snap fitting and the location of one of the guidewire ports.

FIG. 17B more clearly illustrates the features of the catheter system 1700 in FIG. 17A. The catheter system 1700 includes a first catheter 1702 and a second catheter 1730. The first catheter 1702 includes an elongate shaft 1704 with a radially expandable balloon 1706 disposed near a distal end of the elongate shaft 1704. A stent 1708 having a proximal portion 1722, a distal portion 1714 and a side hole 1720 is disposed over the balloon 1706. The distal portion 1714 is crimped to the balloon 1706 to prevent ejection during delivery, while the proximal portion 1722 is partially crimped to the balloon 1706 so the second catheter 1730 may be slidably advanced under the proximal portion 1722 of stent 1708. The first catheter 1702 is an over-the-wire (OTW) catheter having a guidewire lumen 1712 extending from the distal guidewire port 1710 at the distal end of the elongate shaft 1704 to the proximal end of the elongate shaft 1704 into Y-adapter 1713 having a connector 1716. The connector 1716 is for example a Luer connector, and this allows easy coupling with a syringe or other device for lumen flushing or injecting contrast media. When unconnected, the guidewire lumen 1712 exits via connector 1716. A second connector 1718, also for example a Luer connector, allows attachment of an Indeflator or other device to the first catheter 1702 for inflation of the balloon 1706 via an inflation lumen (not shown) in the elongate shaft 1704. The first catheter 1702 also includes a zipper or snap fitting 1724 coupled to the elongate shaft 1704. The snap fitting 1724 may be coextruded with the first shaft 1704, or it may be bonded or otherwise attached thereto using techniques known to those skilled in the art. The snap fitting 1724 may alternatively be coupled with the other shaft 1732. The snap fitting 1724 includes a central channel 1726 extending therethrough and is sized to slidably receive a portion of the second catheter 1730. An elongate slot 1745 extends along the entire length of the snap fitting 1724 and is sized so that shaft 1732 may be snapped into the central channel 1726. The snap fitting 1724 may extend from the distal end of connectors 1714, 1744 to the proximal end of balloon 1706, or it may be shorter, extending only partially between the connectors 1714, 1744 and the balloon 1706. FIG. 17C illustrates a partial cross-section of FIG. 17B taken along the line C-C and shows shaft 1704 with the snap fitting 1724. Radiopaque markers may be placed at different locations along the shaft 1704, often near the balloon 1706 and/or stent 1708, to help mark the proximal and distal ends of the stent 1708 or balloon 1706, as well to facilitate alignment of the two catheters during stent deployment, as discussed elsewhere in this specification.

The second catheter 1730 includes an elongate shaft 1732 with a radially expandable balloon 1740 disposed near a distal end of the elongate shaft 1732. A stent 1742 is disposed over balloon 1740. The stent 1742 may have a length that matches the working length of the balloon 1740, or the stent length may be shorter than the balloon working length. In some examples, the stent 1742 is shorter than the working length of the balloon 1740 so that a proximal portion of the balloon 1740 is unconstrained by the stent 1742, and this unconstrained portion of the balloon 1740 may be slidably advanced or retracted through side hole 1720 and under proximal portion 1722 of stent 1708 as will be discussed below. Stent 1742 is crimped to balloon 1740 to prevent ejection during delivery. At least a portion of balloon 1740 and stent 1742 are distally offset relative to balloon 1706 and stent 1708 so as to minimize profile of the device. In this example the distal stent 1742 may be deployed in a main branch of the vessel and the other stent 1708 may be deployed in a side branch of the vessel. Alternatively, the distal stent 1742 may be deployed in a side branch of a vessel and the other stent 1708 may be deployed in the main branch of a vessel.

The second catheter 1730 is a rapid-exchange catheter (RX) having a guidewire lumen 1734 extending from the distal guidewire port 1738 at the distal end of the elongate shaft 1732 to a proximal guidewire port 1736, which is closer to the distal guidewire port 1738 than the proximal end of the catheter shaft 1732. The proximal guidewire port 1736 is also unobstructed by the snap fitting 1724 and for example distal thereto. A connector 1744, for example a Luer connector, is connected to the proximal end of the elongate shaft 1732 and allows an Indeflator or other device to be coupled with an inflation lumen (not shown) in elongate shaft 1732 for inflation of balloon 1740. A portion of shaft 1732 is snapped into the central channel 1726 of the snap fitting 1724 via slot 1745, and thus shaft 1732 may slide in channel 1726. This helps keep the two catheter shafts 1704, 1732 parallel and prevents tangling during delivery and as shaft 1732 is slidably advanced or retracted relative to shaft 1704. Also, another portion of shaft 1732 is disposed under proximal portion 1722 of stent 1708. The second catheter 1730 may also be slidably advanced or retracted under the proximal portion 1722 of stent 1708 so that the shaft 1732 passes through the side hole 1720 in stent 1708. Radiopaque markers may be placed at different locations on the shaft 1732, often near the balloon 1740 or stent 1742, to help mark the proximal and distal ends of the stent 1742 or balloon 1740, as well to facilitate alignment of the two catheters during stent deployment, as discussed elsewhere in this specification.

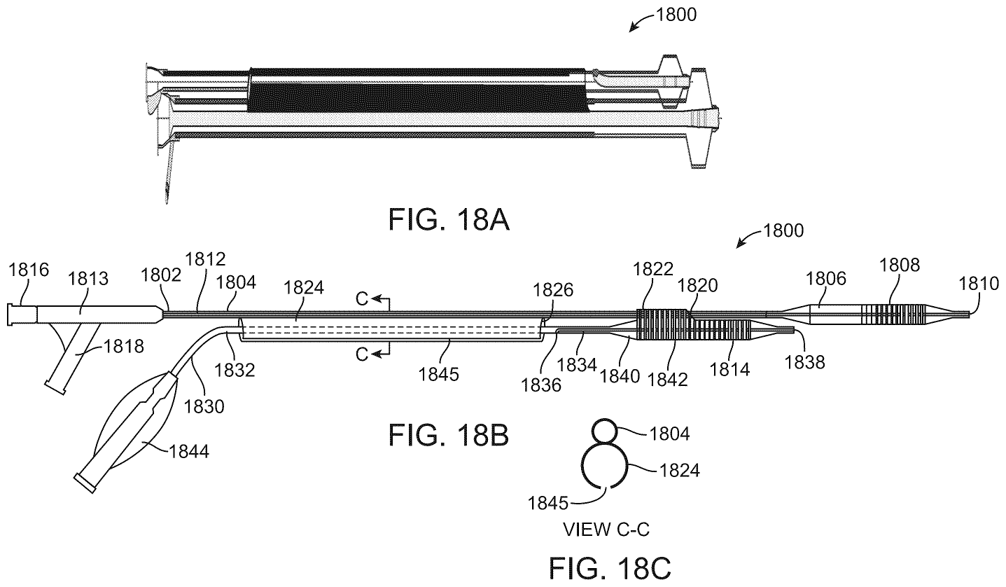

FIG. 18A illustrates a catheter system 1800 having a proximal mother catheter with a rapid-exchange configuration and a distal daughter catheter with an end-to-end zipper or snap fitting. FIG. 18A is similar to the example of FIGS. 14A-14B, with the major difference being the length of the snap fitting and the location of one of the guidewire ports.

FIG. 18B more clearly illustrates the features of the catheter system 1800 in FIG. 18A. The catheter system 1800 includes a first catheter 1802 and a second catheter 1830. The first catheter 1802 includes an elongate shaft 1804 with a radially expandable balloon 1806 disposed near a distal end of the elongate shaft 1804, and a stent 1808 disposed over the balloon 1806. The stent 1808 may be the same length as the working length of the balloon 1806, or it may be shorter. In some examples, the stent 1808 is shorter than the working length of balloon 1806 such that a proximal portion of balloon 1806 remains unconstrained by stent 1808. The proximal portion of balloon 1806 may be slidably advanced and retracted under stent 1842 via side hole 1820. Stent 1808 is crimped to the balloon 1806 to prevent ejection during delivery. The first catheter 1802 is an over-the-wire (OTW) catheter having a guidewire lumen 1812 extending from the distal guidewire port 1810 at the distal end of the elongate shaft 1804 to the proximal end of the elongate shaft 1804 into Y-adapter 1813 having a connector 1816. The connector 1816 is for example a Luer connector, and this allows easy coupling with a syringe or other device for lumen flushing or injecting contrast media. When unconnected, the guidewire lumen 1812 exits via connector 1816. A second connector 1818, also for example a Luer connector, allows attachment of an Indeflator or other device to the first catheter 1802 for inflation of the balloon 1806 via an inflation lumen (not shown) in the elongate shaft 1804. The first catheter 1802 also includes a zipper or snap fitting 1824 coupled to the elongate shaft 1804. The snap fitting 1824 may be coextruded with the first shaft 1804, or it may be bonded or otherwise attached thereto using techniques known to those skilled in the art. The snap fitting 1824 may alternatively be coupled with the other shaft 1832. The snap fitting 1824 includes a central channel 1826 extending therethrough and is sized to slidably receive a portion of the second catheter 1830. An elongate slot 1845 extends along the entire length of the snap fitting 1824 and is sized so that shaft 1832 may be snapped into the central channel 1826. FIG. 18C illustrates a partial cross-section of FIG. 18B taken along the line C-C and shows shaft 1804 with the snap fitting 1824. The snap fitting 1824 may extend from the distal end of connectors 1816, 1844 to the proximal end of balloon 1840, or it may be shorter, extending only partially between the connectors 1816, 1844 and the balloon 1806. Radiopaque markers may be placed at different locations along the shaft 1804, often near the balloon 1806 and/or stent 1808, to help mark the proximal and distal ends of the stent 1808 or balloon 1806, as well as to facilitate alignment of the two catheters during stent deployment, as discussed elsewhere in this specification.

The second catheter 1830 includes an elongate shaft 1832 with a radially expandable balloon 1840 disposed near a distal end of the elongate shaft 1832. A stent 1842 having a proximal portion 1822, a distal portion 1814, and a side hole 1820 is disposed over balloon 1840. The distal portion 1814 is crimped to balloon 1840 to prevent ejection during delivery, while the proximal portion 1822 is partially crimped to balloon 1840 so elongate shaft 1804 may be slidably advanced or retracted under the proximal portion 1822 of stent 1842. The stent 1842 may for example have a length that matches the working length of the balloon 1840, or the stent length may be shorter than the balloon working length. At least a portion of balloon 1806 and stent 1808 are distally offset relative to balloon 1840 and stent 1842 so as to minimize profile of the device. In this example the distal stent 1808 may be deployed in a main branch of the vessel and the other stent 1842 may be deployed in a side branch of the vessel. Alternatively, the distal stent 1808 may be deployed in a side branch of a vessel and the other stent 1842 may be deployed in the main branch of a vessel. The second catheter 1830 is a rapid exchange catheter (RX) having a guidewire lumen 1834 extending from the distal guidewire port 1838 at the distal end of the elongate shaft 1832 to a proximal guidewire port 1836, which is closer to the distal guidewire port 1838 than the proximal end of the catheter shaft 1832. The proximal guidewire port 1836 is also unobstructed by the snap fitting 1824 and for example distal thereto. A connector 1844, for example a Luer connector, is connected to the proximal end of the elongate shaft 1832 and allows an Indeflator or other device to be coupled with an inflation lumen (not shown) in elongate shaft 1832 for inflation of balloon 1840. A portion of shaft 1832 is snapped into the central channel 1826 of the snap fitting 1824 via slot 1845, and thus shaft 1832 may slide in channel 1826. This helps keep the two catheter shafts 1804, 1832 parallel and prevents tangling during delivery and as shaft 1832 is slidably advanced or retracted relative to shaft 1804. Also, a portion of shaft 1804 is disposed under proximal portion 1822 of stent 1842. The first catheter 1802 may be slidably advanced or retracted under the proximal portion 1822 of stent 1842 so that the shaft 1804 passes through the side hole 1820 in stent 1842. Radiopaque markers may be placed at different locations on the shaft 1832, often near the balloon 1840 or stent 1842, to help mark the proximal and distal ends of the stent 1842 or balloon 1840, as well as to facilitate alignment of the two catheters during stent deployment, as discussed elsewhere in this specification.

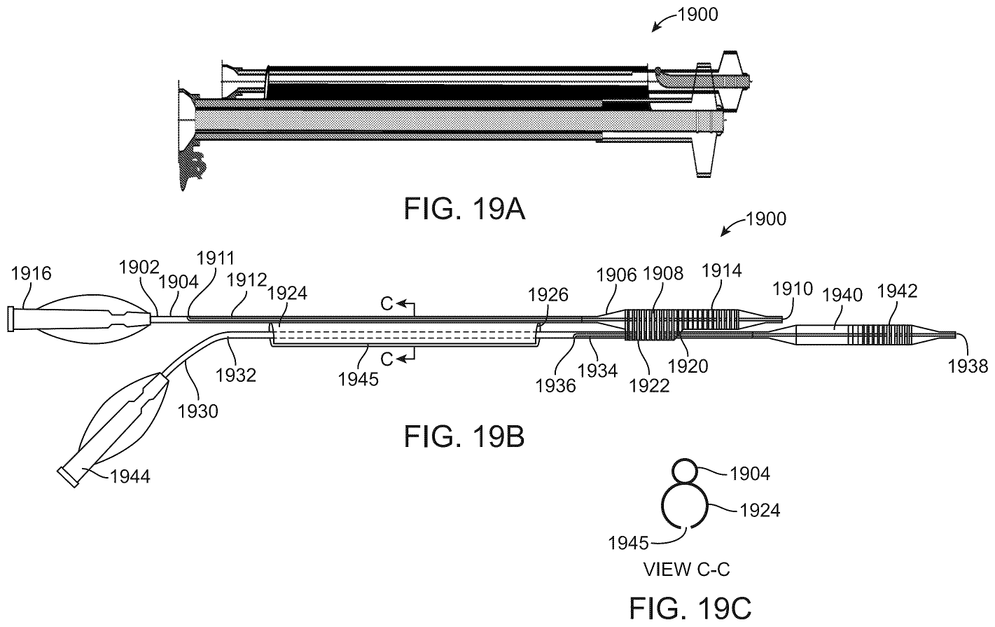

FIG. 19A illustrates a catheter system 1900 having a dual rapid-exchange design with an end-to-end zipper or snap fitting. FIG. 19A is similar to the example of FIGS. 15A-15B, with the major difference being the length of the snap fitting.

FIG. 19B more clearly illustrates the features of the catheter system 1900 in FIG. 19A. The catheter system 1900 includes a first catheter 1902 and a second catheter 1930. The first catheter 1902 includes an elongate shaft 1904 with a radially expandable balloon 1906 disposed near a distal end of the elongate shaft 1904. A stent 1908 having a proximal portion 1922, a distal portion 1914 and a side hole 1920 is disposed over the balloon 1906. The distal portion 1914 is crimped to the balloon 1906 to prevent ejection during delivery, while the proximal portion 1922 is partially crimped to the balloon 1906 so the second catheter 1930 may be slidably advanced under the proximal portion 1922 of stent 1908. The first catheter 1902 is a rapid exchange catheter (RX) having a guidewire lumen 1912 extending from the distal guidewire port 1910 at the distal end of the elongate shaft 1904 to a proximal guidewire port 1911, which is closer to the distal guidewire port 1910 than the proximal end of the catheter shaft 1904. A connector 1916 is coupled with the proximal end of the elongate shaft 1904. The connector 1916 is for example a Luer connector, and this allows easy coupling with an Indeflator or other device for inflation of the balloon 1906. The first catheter 1902 also includes a zipper or snap fitting 1924 coupled to the elongate shaft 1904. The snap fitting 1924 may be coextruded with the first shaft 1904, or it may be bonded or otherwise attached thereto using techniques known to those skilled in the art. The snap fitting 1924 may alternatively be coupled with the other shaft 1932. The snap fitting 1924 includes a central channel 1926 extending therethrough and is sized to slidably receive a portion of the second catheter 1930. An elongate slot 1945 extends along the entire length of the snap fitting 1924 and is sized so that shaft 1932 may snapped into the central channel 1926. FIG. 19C illustrates a partial cross-section of FIG. 19B taken along the line C-C and shows shaft 1904 with the snap fitting 1924. Radiopaque markers may be placed at different locations along the shaft 1904, often near the balloon 1906 and/or stent 1908, to help mark the proximal and distal ends of the stent 1908 or balloon 1906, as well as to facilitate alignment of the two catheters during stent deployment, as discussed elsewhere in this specification.

The second catheter 1930 includes an elongate shaft 1932 with a radially expandable balloon 1940 disposed near a distal end of the elongate shaft 1932. A stent 1942 is disposed over balloon 1940. The stent 1942 may have a length that matches the working length of the balloon 1940, or the stent length may be shorter than the balloon working length. In some examples, the stent 1942 is shorter than the working length of the balloon 1940 so that a proximal portion of the balloon 1940 is unconstrained by the stent 1942 and this unconstrained portion of the balloon 1940 may be slidably advanced or retracted through side hole 1920 and under proximal portion 1922 of stent 1908 as will be discussed below. Stent 1942 is crimped to balloon 1940 to prevent ejection during delivery. At least a portion of balloon 1940 and stent 1942 are distally offset relative to balloon 1906 and stent 1908 so as to minimize profile of the device. In this example the distal stent 1942 may be deployed in a main branch of the vessel and the other stent 1908 may be deployed in a side branch of the vessel. Alternatively, the distal stent 1942 may be deployed in a side branch of a vessel and the other stent 1908 may be deployed in the main branch of a vessel.

The second catheter 1930 is a rapid exchange catheter (RX) having a guidewire lumen 1934 extending from the distal guidewire port 1938 at the distal end of the elongate shaft 1932 to a proximal guidewire port 1936, which is closer to the distal guidewire port 1938 than the proximal end of the catheter shaft 1932. The proximal guidewire port 1936 is also unobstructed by the snap fitting 1924 and may be distal thereto. A connector 1944, for example a Luer connector, is connected to the proximal end of the elongate shaft 1932 and allows an Indeflator or other device to be coupled with an inflation lumen (not shown) in elongate shaft 1932 for inflation of balloon 1940. A portion of shaft 1932 is snapped into the central channel 1926 of the snap fitting 1924 via slot 1945, and thus shaft 1932 may slide in channel 1926. This helps keep the two catheter shafts 1904, 1932 parallel and prevents tangling during delivery and as shaft 1932 is slidably advanced or retracted relative to shaft 1904. Also, another portion of shaft 1932 is disposed under proximal portion 1922 of stent 1908. The second catheter 1930 may also be slidably advanced or retracted under the proximal portion 1922 of stent 1908 so that the shaft 1932 passes through the side hole 1920 in stent 1908. Radiopaque markers may be placed at different locations on the shaft 1932, often near the balloon 1940 or stent 1942, to help mark the proximal and distal ends of the stent 1942 or balloon 1940, as well as to facilitate alignment of the two catheters during stent deployment, as discussed elsewhere in this specification.

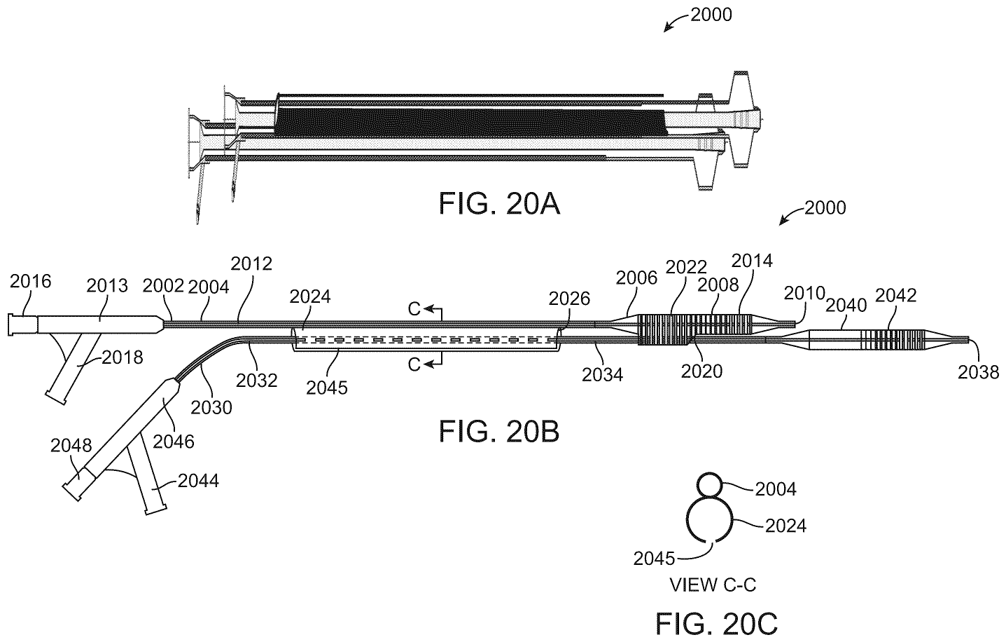

FIG. 20A illustrates a catheter system 2000 having a dual over-the-wire design with an end-to-end zipper or snap fitting. FIG. 20A is similar to the example of FIGS. 16A-16B, with the major difference being the length of the snap fitting.

FIG. 20B more clearly illustrates the features of the catheter system 2000 in FIG. 20A. The catheter system 2000 includes a first catheter 2002 and a second catheter 2030. The first catheter 2002 includes an elongate shaft 2004 with a radially expandable balloon 2006 disposed near a distal end of the elongate shaft 2004. A stent 2008 having a proximal portion 2022, a distal portion 2014 and a side hole 2020 is disposed over the balloon 2006. The distal portion 2014 is crimped to the balloon 2006 to prevent ejection during delivery, while the proximal portion 2022 is partially crimped to the balloon 2006 so the second catheter 2030 may be slidably advanced under the proximal portion 2022 of stent 2008. The first catheter 2002 is an over-the-wire (OTW) catheter having a guidewire lumen 2012 extending from the distal guidewire port 2010 at the distal end of the elongate shaft 2004 to the proximal end of the elongate shaft 2004 into Y-adapter 2013 having a connector 2016. The connector 2016 is for example a Luer connector, and this allows easy coupling with a syringe or other device for lumen flushing or injecting contrast media. When unconnected, the guidewire lumen 2012 exits via connector 2016. A second connector 2018, also for example a Luer connector, allows attachment of an Indeflator or other device to the first catheter 2002 for inflation of the balloon 2006 via an inflation lumen (not shown) in the elongate shaft 2004. The first catheter 2002 also includes a zipper or snap fitting 2024 coupled to the elongate shaft 2004. The snap fitting 2024 may be coextruded with the first shaft 2004, or it may be bonded or otherwise attached thereto using techniques known to those skilled in the art. The snap fitting 2024 may alternatively be coupled with the other shaft 2032. The snap fitting 2024 includes a central channel 2026 extending therethrough and is sized to slidably receive a portion of the second catheter 2030. An elongate slot 2045 extends along the entire length of the snap fitting 2024 and is sized so that shaft 2036 may snapped into the central channel 2026. FIG. 20C illustrates a partial cross-section of FIG. 20B taken along the line C-C and shows shaft 2004 with the snap fitting 2024. Radiopaque markers may be placed at different locations along the shaft 2004, often near the balloon 2006 and/or stent 2008, to help mark the proximal and distal ends of the stent 2008 or balloon 2006, as well as to facilitate alignment of the two catheters during stent deployment, as discussed elsewhere in this specification.

The second catheter 2030 includes an elongate shaft 2032 with a radially expandable balloon 2040 disposed near a distal end of the elongate shaft 2032. A stent 2042 is disposed over balloon 2040. The stent 2042 may have a length that matches the working length of the balloon 2040, or the stent length may be shorter than the balloon working length. In some examples, the stent 2042 is shorter than the working length of the balloon 2040 so that a proximal portion of the balloon 2040 is unconstrained by the stent 2042, and this unconstrained portion of the balloon 2040 may be slidably advanced or retracted through side hole 2020 and under proximal portion 2022 of stent 2008 as will be discussed below. Stent 2042 is crimped to balloon 2040 to prevent ejection during delivery. At least a portion of balloon 2040 and stent 2042 are distally offset relative to balloon 2006 and stent 2008 so as to minimize profile of the device. In this example the distal stent 2042 may be deployed in a main branch of the vessel and the other stent 2008 may be deployed in a side branch of the vessel. Alternatively, the distal stent 2042 may be deployed in a side branch of a vessel and the other stent 2008 may be deployed in the main branch of a vessel.

The second catheter 2030 is an over-the-wire (OTW) catheter having a guidewire lumen 2034 extending from the distal guidewire port 2038 at the distal end of the elongate shaft 2032 to the proximal end of the elongate shaft 2032 into Y-adapter 2046 having a connector 2048. The connector 2048 is for example a Luer connector, and this allows easy coupling with a syringe or other device for lumen flushing or injecting contrast media. When unconnected, the guidewire lumen 2034 exits via connector 2048. A second connector 2044, also for example a Luer connector, allows attachment of an Indeflator or other device to the second catheter 2030 for inflation of the balloon 2040 via an inflation lumen (not shown) in the elongate shaft 2032. A portion of shaft 2032 is snapped into the central channel 2026 of the snap fitting 2024 via slot 2045, and thus shaft 2032 may slide in channel 2026. This helps keep the two catheter shafts 2004, 2032 parallel and prevents tangling during delivery and as shaft 2032 is slidably advanced or retracted relative to shaft 2004. Also, another portion of shaft 2032 is disposed under proximal portion 2022 of stent 2008. The second catheter 2030 may also be slidably advanced or retracted under the proximal portion 2022 of stent 2008 so that the shaft 2032 passes through the side hole 2020 in stent 2008. Radiopaque markers may be placed at different locations on the shaft 2032, often near the balloon 2040 or stent 2042, to help mark the proximal and distal ends of the stent 2042 or balloon 2040, as well to facilitate alignment of the two catheters during stent deployment, as discussed elsewhere in this specification.

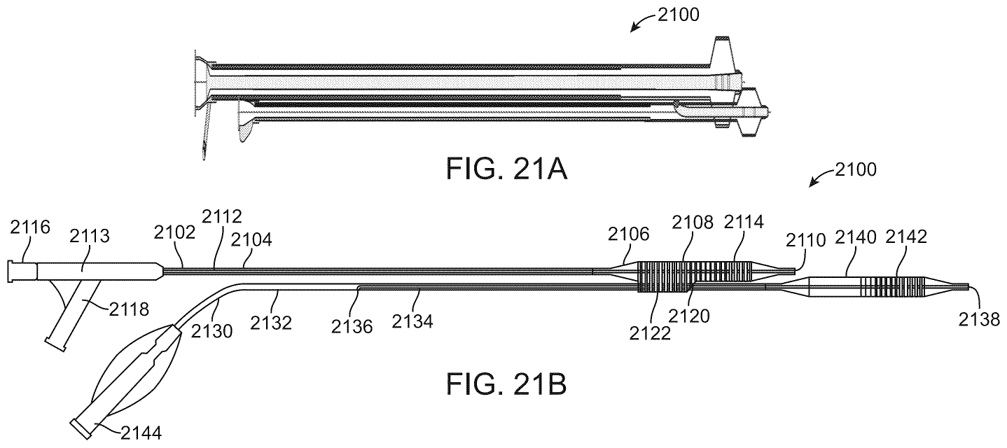

FIGS. 21A, 22A, 23A, and 24A illustrate catheters that can be used with an alternative example where the mother catheter is provided to the operator with a mother stent that is crimped on the distal portion of the mother catheter balloon. The proximal portion of the mother stent is uncrimped or partially crimped. The operator can mount any commercially available catheter or balloon on a wire through the mother stent proximal end and exit out the side hole of the mother stent. The operator can align the catheters to suit the patient's anatomy and crimp the proximal portion of the mother stent. The operator can crimp the stent tightly so that the catheters do not move relative to each other. It is possible for the operator to place the catheters at the bifurcation and, if necessary, pull back on the commercially available catheter to adjust the alignment. Then the operator can gently push the system distally to ensure complete apposition.

FIG. 21A illustrates a catheter system 2100 having a distal daughter catheter with a rapid exchange configuration and a proximal mother catheter with an over-the-wire configuration.

FIG. 21B more clearly illustrates the features of the catheter system 2100 in FIG. 21A. The catheter system 2100 includes a first catheter 2102 and a second catheter 2130. The first catheter 2102 includes an elongate shaft 2104 with a radially expandable balloon 2106 disposed near a distal end of the elongate shaft 2104. A stent 2108 having a proximal portion 2122, a distal portion 2114 and a side hole 2120 is disposed over the balloon 2106. The distal portion 2114 is crimped to the balloon 2106 to prevent ejection during delivery, while the proximal portion 2122 is partially crimped to the balloon 2106 so the second catheter 2130 may be slidably advanced under the proximal portion 2122 of stent 2108. The first catheter 2102 is an over-the-wire (OTW) catheter having a guidewire lumen 2112 extending from the distal guidewire port 2110 at the distal end of the elongate shaft 2104 to the proximal end of the elongate shaft 2104 into Y-adapter 2113 having a connector 2116. The connector 2116 is for example a Luer connector, and this allows easy coupling with a syringe or other device for lumen flushing or injecting contrast media. When unconnected, the guidewire lumen 2112 exits via connector 2116. A second connector 2118, also for example a Luer connector, allows attachment of an Indeflator or other device to the catheter for inflation of the balloon 2106 via an inflation lumen (not shown) in the elongate shaft 2104. Radiopaque markers may be placed at different locations along the shaft 2104, often near the balloon 2106 and/or stent 2108, to help mark the proximal and distal ends of the stent 2108 or balloon 2106, as well as to facilitate alignment of the two catheters during stent deployment, as discussed elsewhere in this specification.

The second catheter 2130 includes an elongate shaft 2132 with a radially expandable balloon 2140 disposed near a distal end of the elongate shaft 2132. A stent 2142 is disposed over balloon 2140. The stent 2142 may have a length that matches the working length of the balloon 2140, or the stent length may be shorter than the balloon working length. In some examples, the stent 2142 is shorter than the working length of the balloon 2140 so that a proximal portion of the balloon 2140 is unconstrained by the stent 2142, and this unconstrained portion of the balloon 2140 may be slidably advanced or retracted through side hole 2120 and under proximal portion 2122 of stent 2108 as will be discussed below. Stent 2142 is crimped to balloon 2140 to prevent ejection during delivery. At least a portion of balloon 2140 and stent 2142 are distally offset relative to balloon 2106 and stent 2108 so as to minimize profile of the device. In this example the distal stent 2142 may be deployed in a main branch of the vessel and the other stent 2108 may be deployed in a side branch of the vessel. Alternatively, the distal stent 2142 may be deployed in a side branch of a vessel and the other stent 2108 may be deployed in the main branch of a vessel.

The second catheter 2130 is a rapid exchange catheter (RX) having a guidewire lumen 2134 extending from the distal guidewire port 2138 at the distal end of the elongate shaft 2132 to a proximal guidewire port 2136, which is closer to the distal guidewire port 2138 than the proximal end of the catheter shaft 2132. A connector 2144, for example a Luer connector, is connected to the proximal end of the elongate shaft 2132 and allows an Indeflator or other device to be coupled with an inflation lumen (not shown) in elongate shaft 2132 for inflation of balloon 2140. Having a portion of shaft 2132 disposed under proximal portion 2122 of stent 2108 helps keep catheter shafts 2104, 2132 parallel and prevents tangling during delivery and as shaft 2132 is slidably advanced or retracted relative to shaft 2104. Also, another portion of shaft 2132 is disposed under proximal portion 2122 of stent 2108. The second catheter 2130 may also be slidably advanced or retracted under the proximal portion 2122 of stent 2108 so that the shaft 2132 passes through the side hole 2120 in stent 2108. Radiopaque markers may be placed at different locations on the shaft 2132, often near the balloon 2140 or stent 2142, to help mark the proximal and distal ends of the stent 2142 or balloon 2140, as well as to facilitate alignment of the two catheters during stent deployment, as discussed elsewhere in this specification.

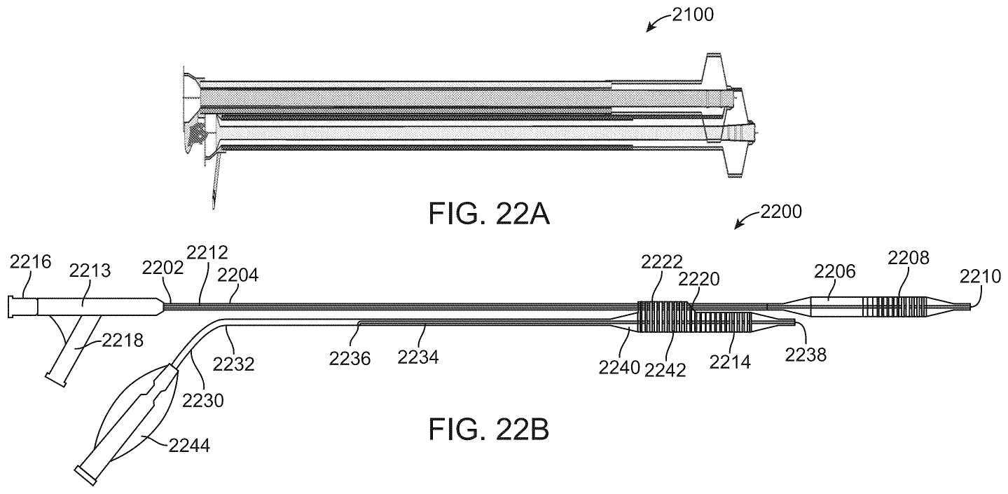

FIG. 22A illustrates a catheter system 2200 having a proximal mother catheter with an over-the-wire design and a distal daughter catheter with an over-the-wire configuration.

FIG. 22B more clearly illustrates the features of the catheter system 2200 in FIG. 22A. The catheter system 2200 includes a first catheter 2202 and a second catheter 2230. The first catheter 2202 includes an elongate shaft 2204 with a radially expandable balloon 2206 disposed near a distal end of the elongate shaft 2204, and a stent 2208 disposed over the balloon 2206. The stent 2208 may be the same length as the working length of the balloon 2206, or it may be shorter. In some examples, the stent 2208 is shorter than the working length of balloon 2206 such that a proximal portion of balloon 2206 remains unconstrained by stent 2208. The proximal portion of balloon 2206 may be slidably advanced and retracted under stent 2242 via side hole 2220. Stent 2208 is crimped to the balloon 2206 to prevent ejection during delivery. The first catheter 2202 is an over-the-wire (OTW) catheter having a guidewire lumen 2212 extending from the distal guidewire port 2210 at the distal end of the elongate shaft 2204 to the proximal end of the elongate shaft 2204 into Y-adapter 2213 having a connector 2216. The connector 2216 is for example a Luer connector, and this allows easy coupling with a syringe or other device for lumen flushing or injecting contrast media. When unconnected, the guidewire lumen 2212 exits via connector 2216. A second connector 2218, also for example a Luer connector, allows attachment of an Indeflator or other device to the catheter for inflation of the balloon 2206 via an inflation lumen (not shown) in the elongate shaft 2204. Radiopaque markers may be placed at different locations along the shaft 2204, often near the balloon 2206 and/or stent 2208, to help mark the proximal and distal ends of the stent 2208 or balloon 2206, as well as to facilitate alignment of the two catheters during stent deployment, as discussed elsewhere in this specification.

The second catheter 2230 includes an elongate shaft 2232 with a radially expandable balloon 2240 disposed near a distal end of the elongate shaft 2232. A stent 2242 having a proximal portion 2222, a distal portion 2214, and a side hole 2220 is disposed over balloon 2240. The distal portion 2214 is crimped to balloon 2240 to prevent ejection during delivery, while the proximal portion 2222 is partially crimped to balloon 2240 so elongate shaft 2204 may be slidably advanced or retracted under the proximal portion 2222 of stent 2242. The stent 2242 may for example have a length that matches the working length of the balloon 2240, or the stent length may be shorter than the balloon working length. At least a portion of balloon 2206, and stent 2208 are distally offset relative to balloon 2240 and stent 2242 so as to minimize profile of the device. In this example the distal stent 2208 may be deployed in a main branch of the vessel and the other stent 2242 may be deployed in a side branch of the vessel. Alternatively, the distal stent 2208 may be deployed in a side branch of a vessel and the other stent 2242 may be deployed in the main branch of a vessel.

The second catheter 2230 is a rapid-exchange catheter (RX) having a guidewire lumen 2234 extending from the distal guidewire port 2238 at the distal end of the elongate shaft 2232 to a proximal guidewire port 2236, which is closer to the distal guidewire port 2238 than the proximal end of the catheter shaft 2232. A connector 2244, for example a Luer connector, is connected to the proximal end of the elongate shaft 2232 and allows an Indeflator or other device to be coupled with an inflation lumen (not shown) in elongate shaft 2232 for inflation of balloon 2240. Having a portion of shaft 2204 disposed under proximal portion 2222 of stent 2208 helps keep catheters 2202, 2232 parallel and prevents tangling during delivery and as shaft 2204 is slidably advanced or retracted relative to shaft 2232. The first catheter 2202 may be slidably advanced or retracted under the proximal portion 2222 of stent 2242 so that the shaft 2204 passes through the side hole 2220 in stent 2242. Radiopaque markers may be placed at different locations on the shaft 2232, often near the balloon 2240 or stent 2242, to help mark the proximal and distal ends of the stent 2242 or balloon 2240, as well to facilitate alignment of the two catheters during stent deployment, as discussed elsewhere in this specification.

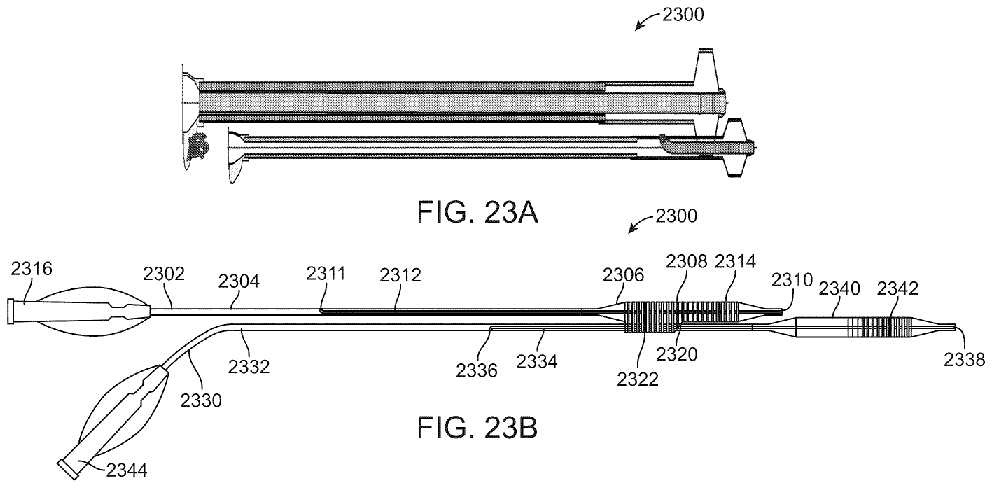

FIG. 23A illustrates a catheter system 2300 having a dual rapid exchange design. FIG. 23B more clearly illustrates the features of the catheter system 2300 in FIG. 23A. The catheter system 2300 includes a first catheter 2302 and a second catheter 2330. The first catheter 2302 includes an elongate shaft 2304 with a radially expandable balloon 2306 disposed near a distal end of the elongate shaft 2304. A stent 2308 having a proximal portion 2322, a distal portion 2314 and a side hole 2320 is disposed over the balloon 2306. The distal portion 2314 is crimped to the balloon 2306 to prevent ejection during delivery, while the proximal portion 2322 is partially crimped to the balloon 2306 so the second catheter 2330 may be slidably advanced under the proximal portion 2322 of stent 2308. The first catheter 2302 is a rapid exchange catheter (RX) having a guidewire lumen 2312 extending from the distal guidewire port 2310 at the distal end of the elongate shaft 2304 to a proximal guidewire port 2311, which is closer to the distal guidewire port 2310 than the proximal end of the catheter shaft 2304. A connector 2316 is coupled with the proximal end of the elongate shaft 2304. The connector 2116 is for example a Luer connector, and this allows easy coupling with an Indeflator or other device for inflation of the balloon 2306. Radiopaque markers may be placed at different locations along the shaft 2304, often near the balloon 2306 and/or stent 2308, to help mark the proximal and distal ends of the stent 2308 or balloon 2306, as well as to facilitate alignment of the two catheters during stent deployment, as discussed elsewhere in this specification.

The second catheter 2330 includes an elongate shaft 2332 with a radially expandable balloon 2340 disposed near a distal end of the elongate shaft 2332. A stent 2342 is disposed over balloon 2340. The stent 2342 may have a length that matches the working length of the balloon 2340, or the stent length may be shorter than the balloon working length. In some examples, the stent 2342 is shorter than the working length of the balloon 2340 so that a proximal portion of the balloon 2340 is unconstrained by the stent 2342, and this unconstrained portion of the balloon 2340 may be slidably advanced or retracted through side hole 2320 and under proximal portion 2322 of stent 2308 as will be discussed below. Stent 2342 is crimped to balloon 2340 to prevent ejection during delivery. At least a portion of balloon 2340 and stent 2342 are distally offset relative to balloon 2306 and stent 2308 so as to minimize profile of the device. In this example the distal stent 2342 may be deployed in a main branch of the vessel and the other stent 2308 may be deployed in a side branch of the vessel. Alternatively, the distal stent 2342 may be deployed in a side branch of a vessel and the other stent 2308 may be deployed in the main branch of a vessel.

The second catheter 2330 is a rapid exchange catheter (RX) having a guidewire lumen 2334 extending from the distal guidewire port 2338 at the distal end of the elongate shaft 2332 to a proximal guidewire port 2336, which is closer to the distal guidewire port 2338 than the proximal end of the catheter shaft 2332. A connector 2344, for example a Luer connector, is connected to the proximal end of the elongate shaft 2332 and allows an Indeflator or other device to be coupled with an inflation lumen (not shown) in elongate shaft 2332 for inflation of balloon 2340. Having a portion of shaft 2332 disposed under proximal portion 2322 of stent 2208 helps keep catheters 2302, 2330 parallel and prevents tangling during delivery and as shaft 2332 is slidably advanced or retracted relative to shaft 2304. The second catheter 2330 may also be slidably advanced or retracted under the proximal portion 2322 of stent 2308 so that the shaft 2332 passes through the side hole 2320 in stent 2308. Radiopaque markers may be placed at different locations on the shaft 2332, often near the balloon 2340 or stent 2342, to help mark the proximal and distal ends of the stent 2342 or balloon 2340, as well as to facilitate alignment of the two catheters during stent deployment, as discussed elsewhere in this specification.

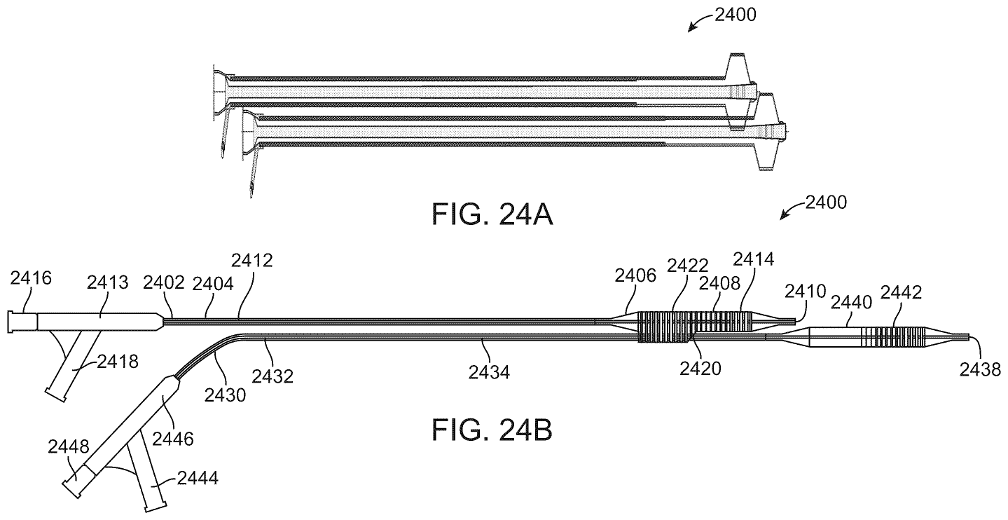

FIG. 24A illustrates a catheter system 2400 having a dual over-the-wire design. FIG. 24B more clearly illustrates the features of the catheter system 2400 in FIG. 24A. The catheter system 2400 includes a first catheter 2402 and a second catheter 2430. The first catheter 2402 includes an elongate shaft 2404 with a radially expandable balloon 2406 disposed near a distal end of the elongate shaft 2404. A stent 2408 having a proximal portion 2422, a distal portion 2414 and a side hole 2420 is disposed over the balloon 2406. The distal portion 2414 is crimped to the balloon 2406 to prevent ejection during delivery, while the proximal portion 2422 is partially crimped to the balloon 2406 so the second catheter 2430 may be slidably advanced under the proximal portion 2422 of stent 2408. The first catheter 2402 is an over-the-wire (OTW) catheter having a guidewire lumen 2412 extending from the distal guidewire port 2410 at the distal end of the elongate shaft 2404 to the proximal end of the elongate shaft 2404 into Y-adapter 2413 having a connector 2416. The connector 2416 is for example a Luer connector, and this allows easy coupling with a syringe or other device for lumen flushing or injecting contrast media. When unconnected, the guidewire lumen 2412 exits via connector 2416. A second connector 2418, also for example a Luer connector, allows attachment of an Indeflator or other device to the catheter for inflation of the balloon 2406 via an inflation lumen (not shown) in the elongate shaft 2404. Radiopaque markers may be placed at different locations along the shaft 2404, often near the balloon 2406 and/or stent 2408, to help mark the proximal and distal ends of the stent 2408 or balloon 2406, as well as to facilitate alignment of the two catheters during stent deployment, as discussed elsewhere in this specification.

The second catheter 2430 includes an elongate shaft 2432 with a radially expandable balloon 2440 disposed near a distal end of the elongate shaft 2432. A stent 2442 is disposed over balloon 2440. The stent 2442 may have a length that matches the working length of the balloon 2440, or the stent length may be shorter than the balloon working length. In some examples, the stent 2442 is shorter than the working length of the balloon 2440 so that a proximal portion of the balloon 2440 is unconstrained by the stent 2442, and this unconstrained portion of the balloon 2440 may be slidably advanced or retracted through side hole 2420 and under proximal portion 2422 of stent 2408 as will be discussed below. Stent 2442 is crimped to balloon 2440 to prevent ejection during delivery. At least a portion of balloon 2440 and stent 2442 are distally offset relative to balloon 2406 and stent 2408 so as to minimize profile of the device. In this example the distal stent 2442 may be deployed in a main branch of the vessel and the other stent 2408 may be deployed in a side branch of the vessel. Alternatively, the distal stent 2442 may be deployed in a side branch of a vessel and the other stent 2408 may be deployed in the main branch of a vessel.

The second catheter 2430 is an over-the-wire (OTW) catheter having a guidewire lumen 2434 extending from the distal guidewire port 2438 at the distal end of the elongate shaft 2432 to the proximal end of the elongate shaft 2432 into Y-adapter 2446 having a connector 2448. The connector 2448 is for example a Luer connector, and this allows easy coupling with a syringe or other device for lumen flushing or injecting contrast media. When unconnected, the guidewire lumen 2434 exits via connector 2448. A second connector 2444, also for example a Luer connector, allows attachment of an Indeflator or other device to the catheter for inflation of the balloon 2440 via an inflation lumen (not shown) in the elongate shaft 2432. Having a portion of shaft 2432 disposed under proximal portion 2422 of stent 2408 helps keep catheters 2402, 2430 parallel and prevents tangling during delivery and as shaft 2432 is slidably advanced or retracted relative to shaft 2404. The second catheter 2430 may also be slidably advanced or retracted under the proximal portion 2422 of stent 2408 so that the shaft 2432 passes through the side hole 2420 in stent 2408. Radiopaque markers may be placed at different locations on the shaft 2432, often near the balloon 2440 or stent 2442, to help mark the proximal and distal ends of the stent 2442 or balloon 2440, as well as to facilitate alignment of the two catheters during stent deployment, as discussed elsewhere in this specification.

In any of the examples disclosed herein, commercially available catheters and commercially available stents may be matched up to form the systems illustrated. In still other examples, commercially available catheters that are single-use devices for treating a single vessel may be mated together in various combinations and coupled together with a polymer sleeve. The operator chooses the two catheters for the patient's anatomy then slides a sized polymer sleeve over both catheters from the distal ends. Once the operator has the catheters aligned, the polymer sleeve can be treated with a heat or light source to shrink and bond the two catheters together with friction. The polymer sleeve is made of typical polymers that can act as shrink wrap when treated with a heat or light source. The polymer of the polymer sleeve for example could be manufactured with polyolefin, a chemical used in manufacturing shrink wrap. The polymer sleeve would not crosslink or covalently attach to the catheters; several types of polymers are commercially available and have the requisite properties—thin, strong, not adhesive—and reaction times to their source of ten minutes or less. The polymer sleeves are typically 15 centimeters in length and have various diameters to suit typical catheter diameters 4 French to 20 French. The operator can test that the bond is holding by applying slight pressure prior to the procedure. If the polymer sleeve does not hold tightly, the operator may elect to use a smaller-diameter polymer sleeve or use more than one polymer sleeve by placing the polymer sleeves adjacent to each other. Alternatively, several smaller sleeves from 1 to 10 centimeters in length could be placed over several different portions of the catheters.

In any of the examples discussed herein, a therapeutic agent may be disposed on the stent or balloon and eluted therefrom in a controlled manner into the target treatment area such as a stenotic lesion. Example therapeutic agents help inhibit restenosis, hyperplasia or have other therapeutic benefits. Example anti-hyperplasia agents include anti-neoplastic drugs, such as paclitaxel, methotrexate, and batimastal; antibiotics such as doxycycline, tetracycline, rapamycin, everolimus, biolimus A9, novolimus, myolimus, zotarolimus, and other analogs and derivatives of rapamycin and actinomycin; immuno-suppressants such as dexamethasone and methyl prednisolone; nitric oxide sources such as nitroprussides; estrogen; estradiols; and the like. Methods for applying the therapeutic agent to the stent or balloon are well known to those skilled in the art and have been described in the patent and scientific literature.

### Stent Delivery:

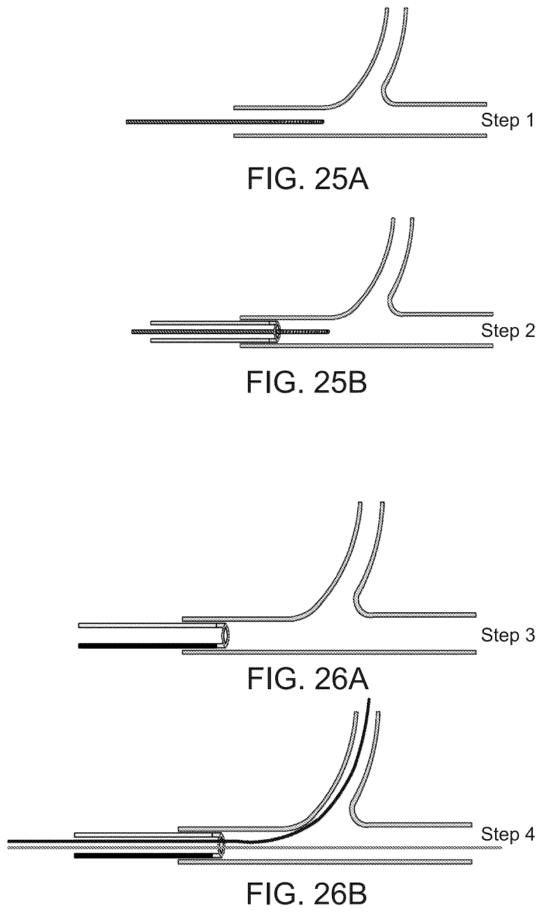

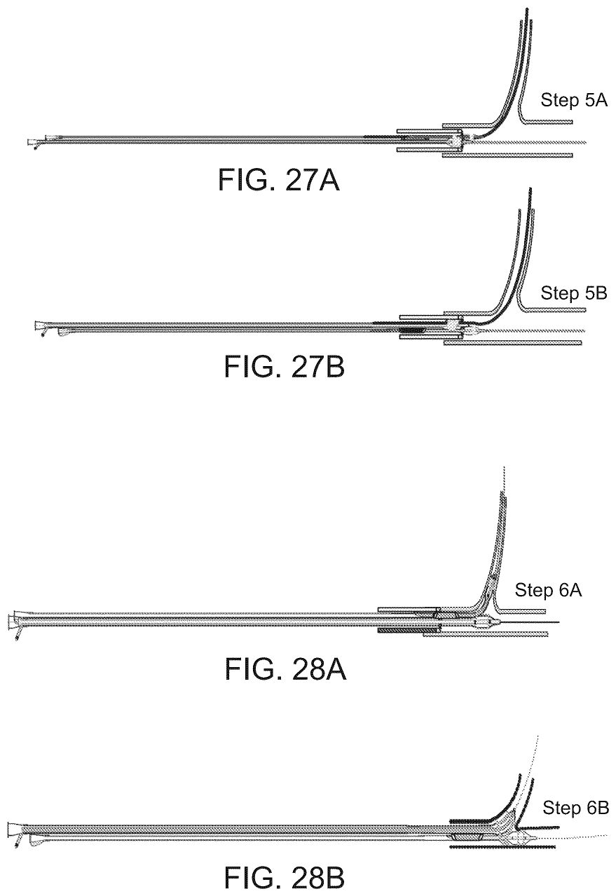

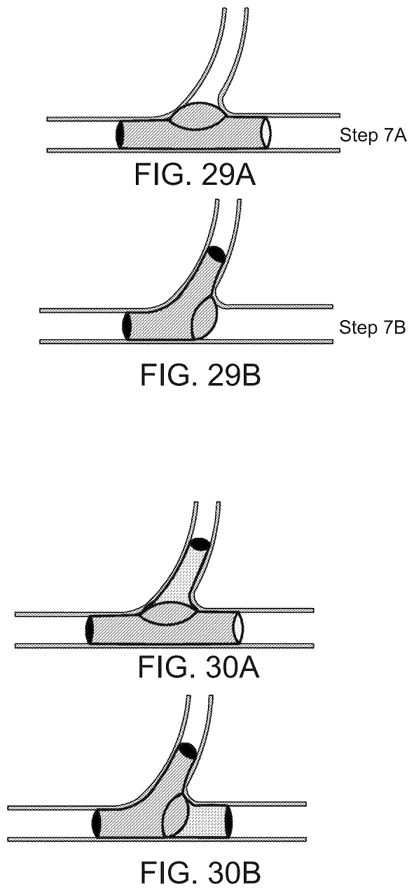

FIGS. 25A-30B illustrate an example delivery sequence of eight steps in some examples. Step 1 illustrates the introduction of a 0.035-inch guidewire up to the bifurcation. Step 2 illustrates the tracking of a guide catheter over the guidewire. Step 3 illustrates the removal of the guidewire and placement position of the guide catheter. Step 4 illustrates the tracking and placement of a rapid-exchange compatible wire in the daughter vessel and an over-the-wire compatible wire in the mother vessel. Steps 5A & 5B illustrate tracking of the catheter system distally over both the guidewires. Step 6A illustrates the inflation of the daughter balloon and placement of the daughter stent and partial deployment of the mother stent. Step 6B illustrates the inflation of the mother balloon to place the distal portion of the mother stent in the mother vessel. Step 7A illustrates the mother stent in the main branch with side hole facing the daughter vessel. Step 7B illustrates the bifurcated stent partially in the daughter vessel and daughter ostium completely opened and continuing on to the mother vessel.

In an alternative example, the delivery catheter mother balloons have tapered ends to accommodate balloons and stents with non-uniform profiles. For example, the proximal end of the daughter vessel stent may be designed to have a larger circumference than the distal end to compensate for the natural bifurcation anatomy. The daughter vessel balloon would likewise have a taper to properly expand the stent and ensure complete apposition. Additionally, it is possible to design the mother stent to expand differentially along its profile to compensate for a larger arterial diameter at the carina or ostium. In other words, the proximal and distal ends of the mother vessel balloon and mother vessel stent would be smaller in circumference while the center portion of the mother vessel stent would have a larger circumference. In an alternative example, the mother vessel balloon has tapered ends to accommodate the distal balloon catheter portion and guidewire lumen. Further, the mother vessel balloon may be designed for differential expansion to accommodate natural vessel anatomy.

In some examples, the distal (daughter) balloon catheter portion is crimped with a half stent on a rapid-exchange catheter. The daughter vessel stent is about 4-20 millimeters long and the daughter vessel balloon is approximately twice as long in length. The mother vessel stent is about 10-30 millimeters long and is differentially crimped to allow independent operation of the daughter balloon catheter portion. The distal portion of the mother vessel stent is crimped tightly enough to keep the entire stent from unintentionally dislodging during the procedure. The proximal portion of the mother vessel stent is crimped just tightly enough to reduce the crossing profile and to allow the daughter balloon catheter portion to be moved distal or proximal relative to the mother balloon catheter portion. The proximal (mother) balloon catheter portion is an over-the-wire type design with the mother vessel balloon for example about 3 centimeters proximal to the daughter vessel balloon. In an alternative example, a stent is designed to allow differential expansion of the middle portion of the stent relative to the proximal and distal ends. In particular, the design facilitates the placement of the stent across a bifurcation lesion in the mother vessel because it has a larger circumference in the middle portion relative to the ends than a stent with a constant profile. Further, the profile can be adjusted so that the largest circumference can be placed proximal or distal to the midpoint of the stent. In the particular example, the largest circumference is distal to the midpoint of the stent but could be easily reversed for variable patient anatomy. Partial crimping has the following features that make it possible to maintain sufficient stent retention during delivery and placement and still allows the secondary system adjustability and deliverability.

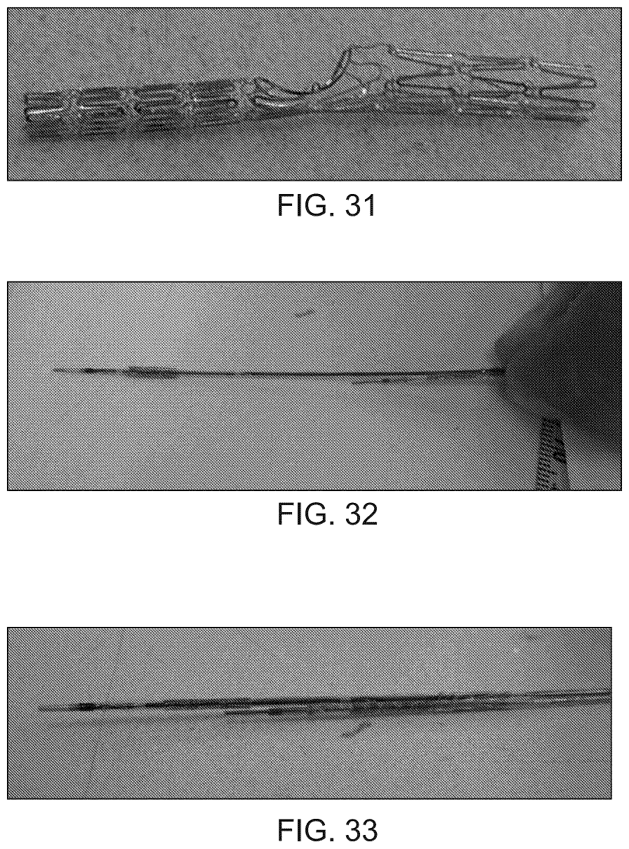

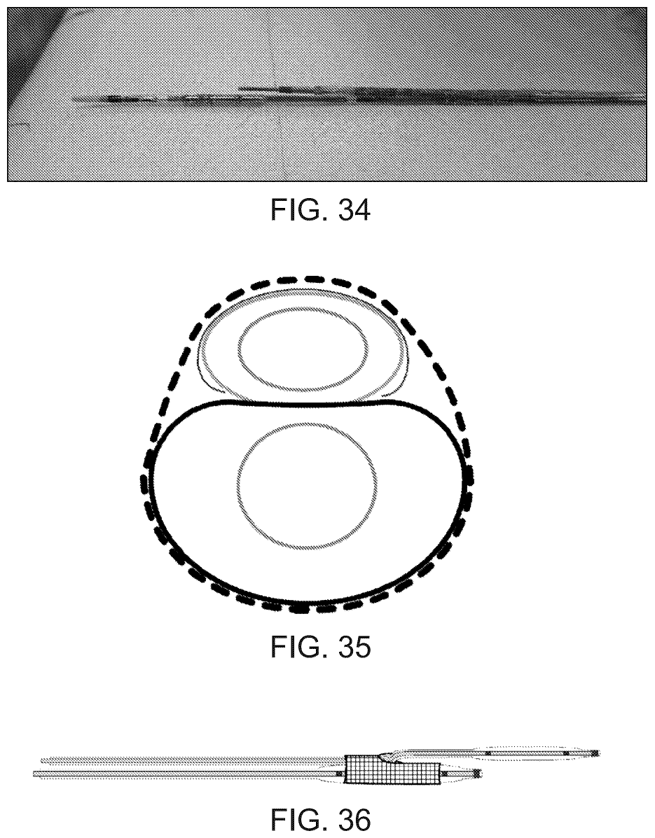

FIG. 31 shows a partially crimped bifurcation stent prior to placement on any balloon catheter. FIGS. 32-34 illustrate an example of the present inventive subject matter in three steps. First, the bifurcation stent is partially crimped over approximately one-third of its distal portion onto the mother catheter balloon, and the daughter catheter is loaded through the mother catheter and mother stent where the daughter stent can be crimped separately. Second, the daughter stent is crimped and pulled back proximally to align the daughter stent proximal end near the mother stent distal end. Third and final, the proximal portion of the mother stent can be crimped to reduce the outer diameter yet still allow independent movement of the two catheters relative to each other.

FIG. 35 illustrates a cross-section of a mother and daughter balloon catheter system without a daughter stent. The daughter catheter is on top of the mother catheter. The mother stent is differentially crimped around the mother catheter balloon and daughter catheter because the daughter catheter profile is smaller than the mother catheter. The differential crimping is non-uniform and can create various cross-sectional shapes to accommodate different catheter designs, balloon designs, and stent designs. For example, pear-shaped or a figure-eight are possible configurations. The current example is designed to reduce the profile as much as possible. In one example method of manufacturing, a protective sheet is placed between the two catheters. The protective sheet only needs to cover the portions that will come in contact during the crimping process; then the protective sheet can be removed.

FIG. 36 illustrates a side view of the mother stent mounted on the mother catheter balloon and the daughter catheter mounted on the mother catheter through the mother stent. The distal portion of the mother stent will be crimped under standard conditions to hold the stent firmly to the mother balloon and mother catheter. The proximal portion of the mother stent is partially crimped to reduce the profile but still allows the daughter catheter freedom to move proximal or distal relative to the mother catheter. This example illustrates that the stent is differentially crimped in both the circumferential and longitudinal directions. The amount of crimping will be determined by the stent design and size, catheter dimensions, and balloon dimensions; thus, the crimping is differential along the longitudinal axis.

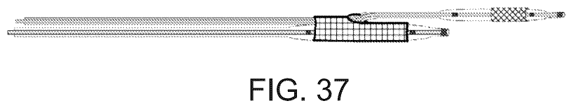

FIG. 37 illustrates a side view of the mother stent mounted on the mother catheter balloon and the daughter catheter mounted on the mother catheter through the mother stent. The daughter catheter also includes a stent that can be crimped under standard conditions. The distal portion of the mother stent will be crimped under standard conditions to hold the stent firmly to the mother balloon and mother catheter. In one experiment, this arrangement was tested to determine the strength of the distal crimping of the mother stent by pulling the daughter catheter and stent proximally; the results were that the daughter catheter successfully passed through the crimped mother stent and still retained the daughter stent as well. Additional features may be utilized during the crimping process such as adding a slight positive internal pressure to the balloon so that the final balloon surface pillows about 0.002 inch beyond the outer diameter of the stent. This process can yield a design that protects the stent from engaging with the vessel, thus reducing friction and improving stent retention at the same time.

Further, this process improves safety and reduces trauma to the vessel. While the above example discloses a bifurcation stent that is crimped at or about its distal half, this is not a limitation. The stent could be differentially crimped along its axis depending upon stent design, such as, for example, if a hole in the side of a stent was not centered along the axis. It may be convenient to have the distal crimped portion of the bifurcation stent extend just distal of the hole that the daughter catheter passes through. Alternatively, the distal crimped portion could extend partially or entirely over the hole that the daughter catheter passes through.

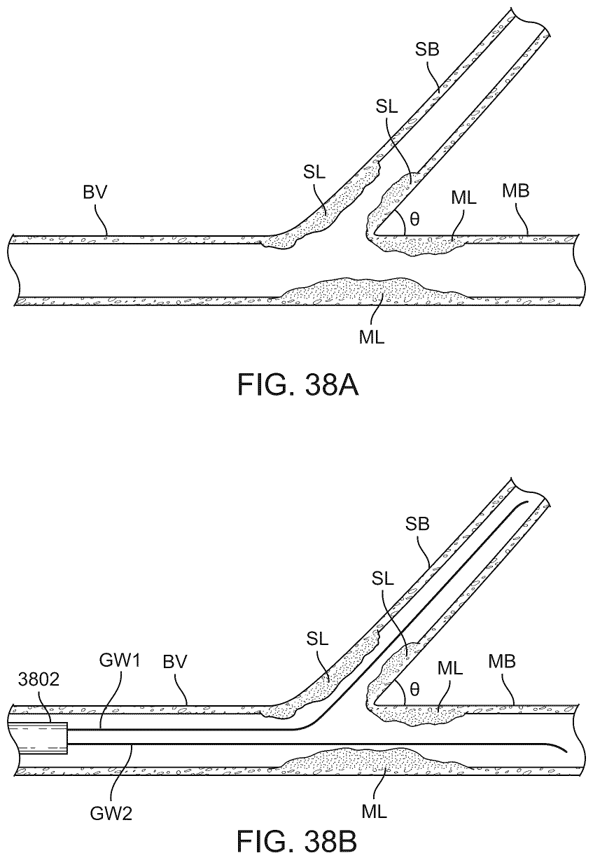

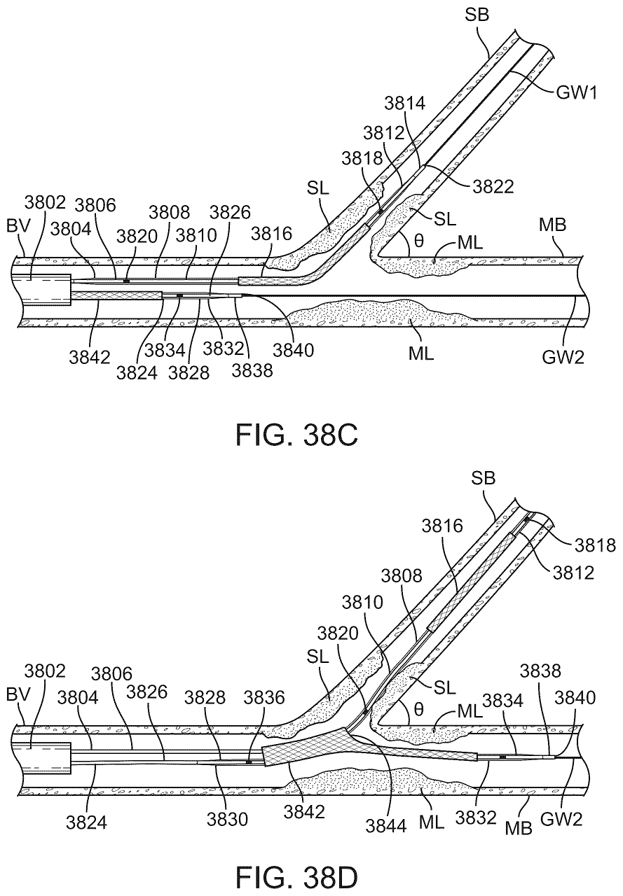

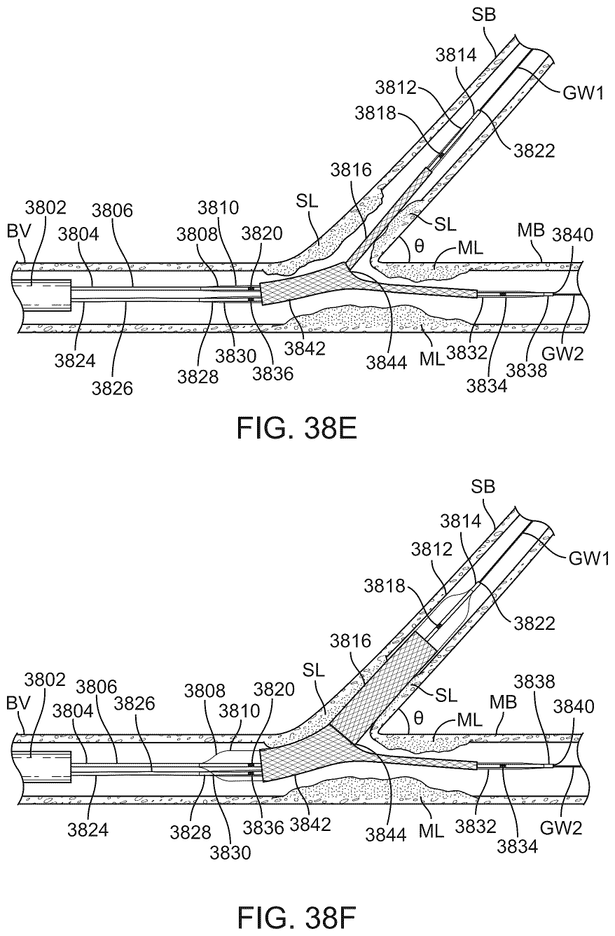

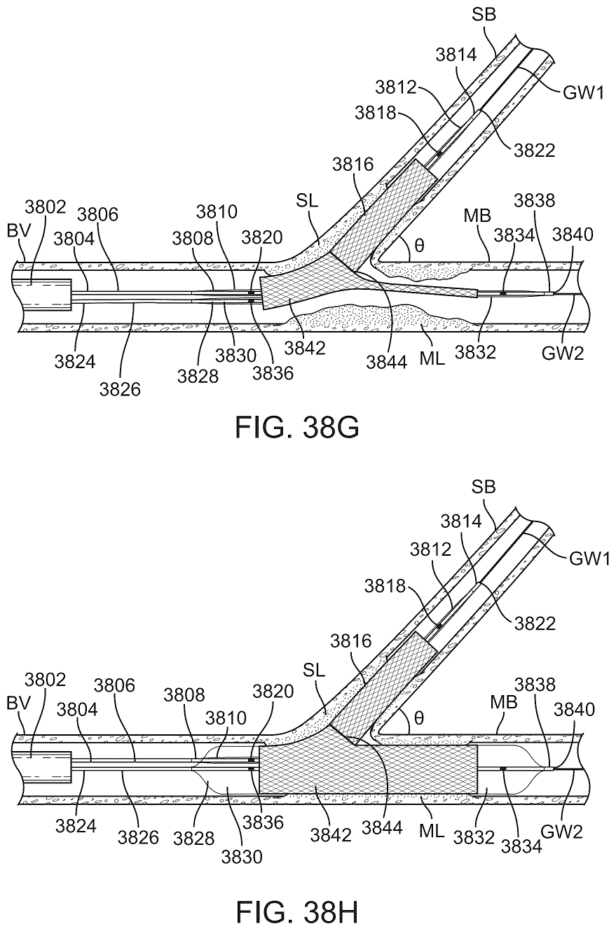

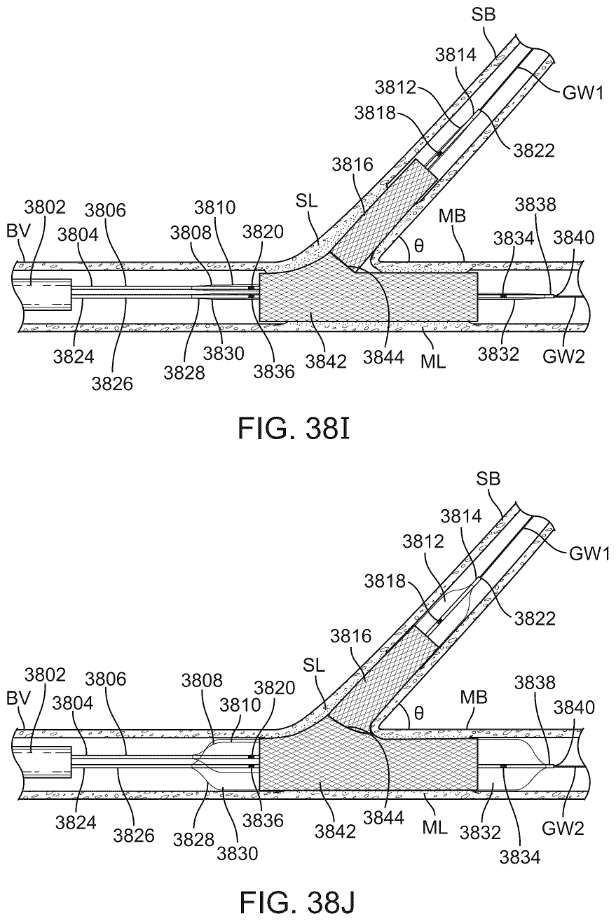

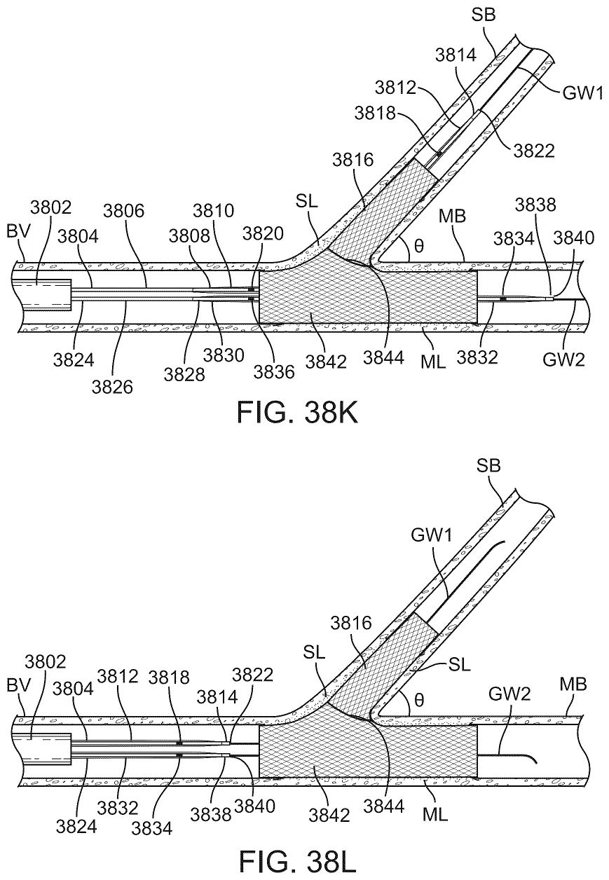

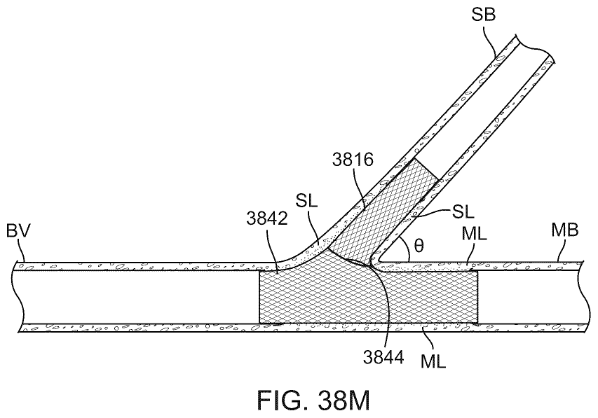

FIGS. 38A-38M more clearly illustrate an example of treating a bifurcated vessel such as a bifurcated coronary artery. In FIG. 38A the bifurcated vessel BV includes a side branch vessel SB and a main branch vessel MB. The main branch has a main branch lesion ML, and the side branch has a side branch lesion SL. The angle between the side branch and the main branch is referred to as the bifurcation angle, and is indicated by θ. When the bifurcation angle θ is less than about 60 to 70 degrees, the distal-most stent of the system can be effectively positioned in the side branch. However, when the bifurcation angle is greater than or equal to about 60 to 70 degrees, it becomes more challenging to position the distal-most stent in the side branch. Moreover, when the distal stent is retracted proximally toward the stent having the side hole (discussed below), the catheter shaft may bind against the side hole, resulting in damage to the catheter shaft and/or stent. Therefore, in some examples, when the bifurcation angle is less than about 60 to 70 degrees, the distal-most stent is for example positioned in the side branch and the proximal-most stent is advanced into the main branch. When the bifurcation angle is greater than or equal to about 60 to 70 degrees, the distal-most stent is positioned in the main branch and the other stent is positioned partially in the main branch and partially in the side branch. This is not intended to limit the use of the catheter system, and either stent may be placed in either side branch or main branch depending on operator preference.

In FIG. 38B, a guide catheter 3802 is advanced distally until its distal end is adjacent the bifurcation. A pair of guidewires GW1, GW2 are then advanced from the guide catheter 3802 distally toward the bifurcation such that the first guidewire GW1 is advanced into the side branch SB and so that the distal tip of the first guidewire GW1 is distal of the side branch lesion SL. Similarly, the second guidewire GW2 is also advanced distally in the main branch MB until the distal tip of the second guidewire GW2 is distal of the main branch lesion ML.

In FIG. 38C, a stent delivery system having a first catheter 3804 and a second catheter 3824 are advanced distally from the guide catheter 3802 toward the bifurcation. The first catheter 3804 includes an elongate catheter shaft 3806 and a radially expandable balloon 3808 disposed over a distal portion of elongate shaft 3806. A balloon-expandable stent 3816 is disposed over the balloon 3808. In this example, the stent 3816 is shorter than the working length of the balloon 3808; therefore, a proximal portion 3810 of the balloon 3808 and a distal portion 3812 are unconstrained by the stent 3816. The proximal portion 3810 may be retracted under a portion of the second stent 3842 and thus when balloon 3808 is inflated, it will radially expand stent 3816 and a portion of stent 3842. However, this is not intended to be limiting, and the stent length may be substantially equal to the working length of the balloon 3808, or it may have shorter length as previously discussed. Proximal radiopaque marker 3820 and distal radiopaque marker 3818 help define proximal and distal ends of the stent 3816 as well as proximal and distal ends of the balloon 3808. The radiopaque markers 3820, 3818 will also be used to help align the two catheters during treatment of the bifurcation, as will be discussed below. The distal tip 3814 may be a soft durometer polymer thereby minimizing trauma to the vessel during delivery. A distal guidewire port 3822 extends from the distal tip 3814 and allows guidewire GW1 to exit or enter a guidewire lumen (not shown) in the elongate shaft 3806. The first catheter 3804 may be a rapid exchange catheter or an over-the-wire catheter, examples of which have been disclosed above. The second catheter 3824 (best seen in FIG. 38D) includes an elongate catheter shaft 3826 with a radially expandable balloon 3828 disposed over a distal region of the elongate shaft 3826. A stent 3842 having a side hole 3844 is disposed over the balloon 3828. The length of the stent 3842 may be substantially the same as the working length of the balloon 3828 or it may be less than the working length. In this example, the stent 3842 has a length shorter than the working length of the balloon 3828, thus a proximal portion 3830 and a distal portion 3832 remain unconstrained by the stent 3842. Proximal radiopaque marker 3836 and distal radiopaque marker 3834 help define the proximal and distal ends of the stent 3842 as well as the proximal and distal ends of the balloon 3828. The radiopaque markers 3836, 3834 will also be used to help align the two catheters during treatment of the bifurcation, as will be discussed below. The distal tip 3838 may be a soft durometer polymer thereby minimizing trauma to the vessel during delivery. A distal guidewire port 3840 extends from the distal tip 3838 and allows guidewire GW2 to exit or enter a guidewire lumen (not shown) in the elongate shaft 3826. The second catheter 3824 may be a rapid exchange catheter or an over-the-wire catheter, examples of which have previously been disclosed above.

Referring back to FIG. 38C, the bifurcation angle is less than about 60 to 70 degrees, and the first catheter 3804 and the second catheter 3824 are further advanced distally so that the first catheter 3804 tracks over the first guidewire GW1 into the side branch SB while the second catheter 3824 tracks over the second guidewire GW2 in the main branch MB toward the main branch lesion ML. Because the first catheter 3804 is coupled with the second catheter 3824 via stent 3842, both catheters are advanced distally simultaneously thereby reducing procedure time, although this is not meant to be limiting, as each catheter may be advanced independently of the other. In this example, the first balloon 3808 and first stent 3816 are distal to the second balloon 3828 and second stent 3842. This axial offset minimizes the system profile.

In FIG. 38D, both catheters 3804, 3824 are advanced further distally toward the bifurcation until the first stent 3816 is distal to the side branch lesion SL and the second stent 3842 traverses the main branch lesion ML and the side hole 3844 is adjacent the ostium of the side branch SB. Advancement of both catheters 3804, 3824 is again performed simultaneously, although they could also be advanced independently of one another. The operator will feel resistance against further advancement of the catheters 3804, 3824 because as the catheters are advanced further distally, the two catheter shafts 3806, 3826 will spread apart relative to one another as they are forced against the carina of the bifurcation. However, a portion of the first elongate shaft 3806 is disposed under a portion of the second stent 3842, therefore the two shafts 3806, 3826 can only spread apart so far. Thus, when an operator feels resistance against further advancement of the catheter shafts, the operator knows that both catheters 3804, 3824 and their associated stents and balloons are properly positioned relative to the bifurcation.

In FIG. 38E, the first catheter 3804 is retracted proximally relative to the second catheter 3824. Because a portion of the first catheter shaft 3806 is disposed under a portion of the second stent 3842, the first shaft 3806 is slidably retracted into side hole 3844 and the first shaft 3806 and proximal portion 3810 of balloon 3808 are slidably retracted under a portion of second stent 3842. The first shaft 3806 is proximally retracted until proximal radiopaque marker 3820 lines up with proximal radiopaque marker 3836 so that a proximal end of the first stent 3816 will be aligned with the side hole 3844 in the second stent 3842. An operator may feel resistance during retraction of the first elongate shaft 3806 relative to the second elongate shaft 3826 when the ends of the stents 3816, 3842 engage one another. Stent 3842 has a distal portion crimped to balloon 3828 to prevent ejection during delivery, and a proximal portion is partially crimped thereto or uncrimped to allow catheter 3804 to slide thereunder. Crimping of the stent is disclosed in greater detail in U.S. patent applications previously incorporated by reference above. The ends of the stents may butt up against one another, overlap with one another, interleave with one another, or combinations thereof. Additional details related to the engagement of the stents is disclosed in U.S. patent applications previously incorporated by reference above. Both stents 3816, 3842 are disposed adjacent their respective lesions SL, ML, and the side hole 3844 is in rough alignment with the ostium to the side branch SB and the side branch stent 3816.

In FIG. 38F, the balloon 3808 is radially expanded, often with contrast medium, saline, or a combination thereof, thereby radially expanding the first stent 3816 into engagement with the side branch lesion SL and the walls of the side branch. A proximal portion 3810 and a distal portion 3812 of the balloon 3808 will also expand; thus, a proximal portion of the second stent 3842 will also be radially expanded. Expansion of the stents occurs simultaneously. Since a portion of balloon 3808 also passes through side hole 3844, expansion of balloon 3808 also partially expands the side hole 3844 and also aligns the side hole 3844 with the ostium of the side branch.

In FIG. 38G the balloon 3808 is contracted, and then in FIG. 38H the other balloon 3828 is radially expanded, with contrast medium, saline, or a combination thereof, thereby further radially expanding the second stent 3842. Expansion of balloon 3828 expands the proximal portion of the stent 3842 into engagement with the main branch vessel wall and main branch lesion ML, and the distal portion of the stent 3842 is also radially expanded into the main branch vessel wall as well as the main branch lesion ML. The side hole 3844 is also further aligned with the ostium of the side branch SB.

Referring now to FIG. 38I, balloon 3828 is contracted and then both balloons are simultaneously inflated in a “kissing balloon” technique as seen in FIG. 38J. Both balloons 3808, 3828 are inflated with contrast medium, saline, or combinations thereof until they engage one another and are fully expanded in the main branch MB and side branch SB. The kissing balloon technique ensures that both stents 3816, 3842 are fully expanded and in full apposition with their respective vessel wall and lesion. Additionally, the kissing balloon technique lines up the proximal end of the first stent 3816 with the side hole 3844 in the second stent 3842, thereby ensuring that continuous and smooth scaffolding from the main branch MB into the side branch SB. Also, the kissing balloons technique ensures that the side hole 3844 does not block the ostium to the side branch thereby avoiding “stent jailing,” or disrupting blood flow into the side branch.

In FIG. 38K, both balloons 3808, 3828 are contracted, and in FIG. 38L both catheters 3804, 3824 are retracted proximally. The catheters 3804, 3824 may be retracted simultaneously or independently of one another. The first catheter 3804 is retracted through both stents 3816, 3842 and also passes through the side hole 3844. The second catheter 3824 is retracted through the second stent 3842. In FIG. 38M, both catheters 3804, 3824 have been removed, as well as the guide catheter 3802 and both guidewires GW1, GW2. Stents 3816, 3842 remain implanted at the bifurcation. Optionally, the stents or balloons may contain therapeutic agents such as those previously discussed, and these may elute out into the lesion at a controlled rate in order to help prevent restenosis.

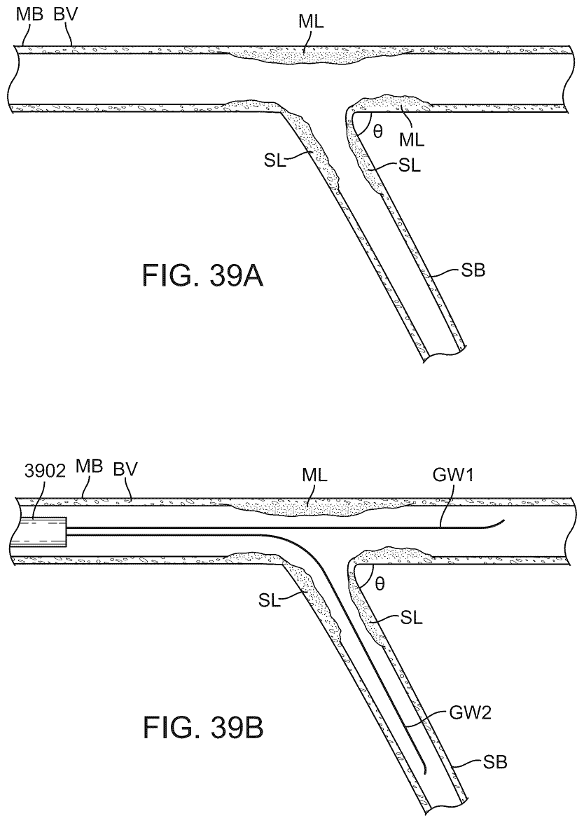

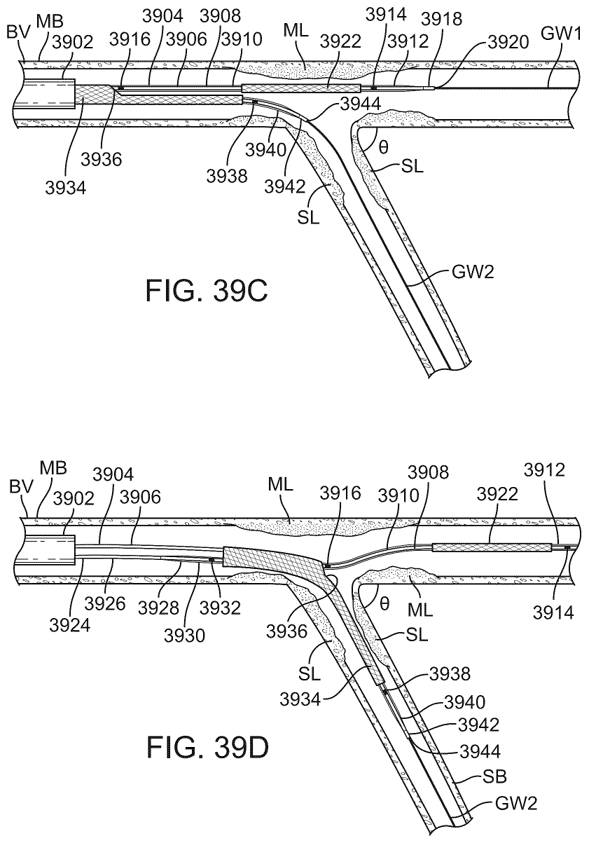

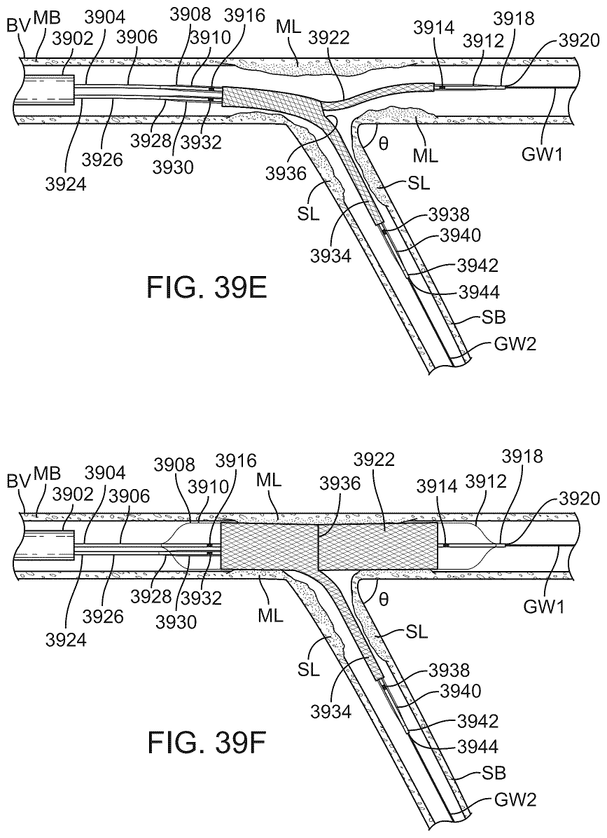

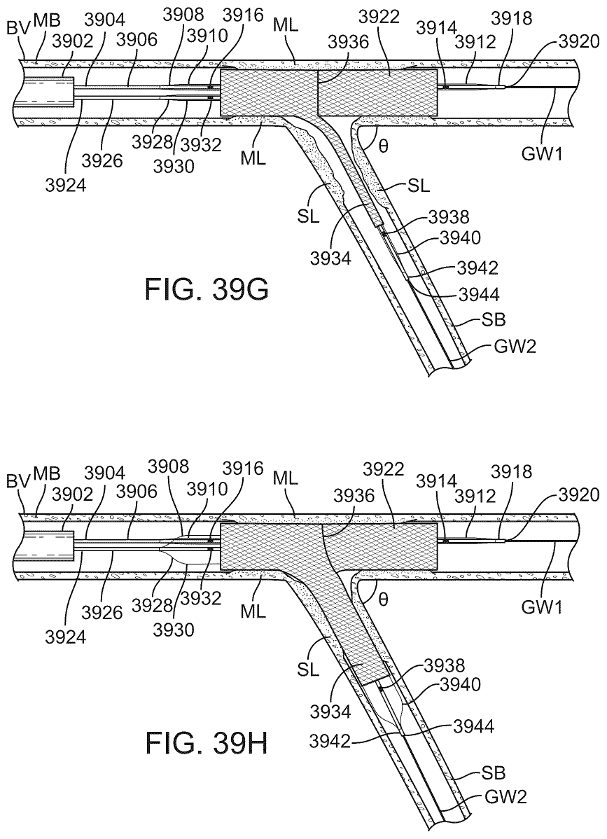

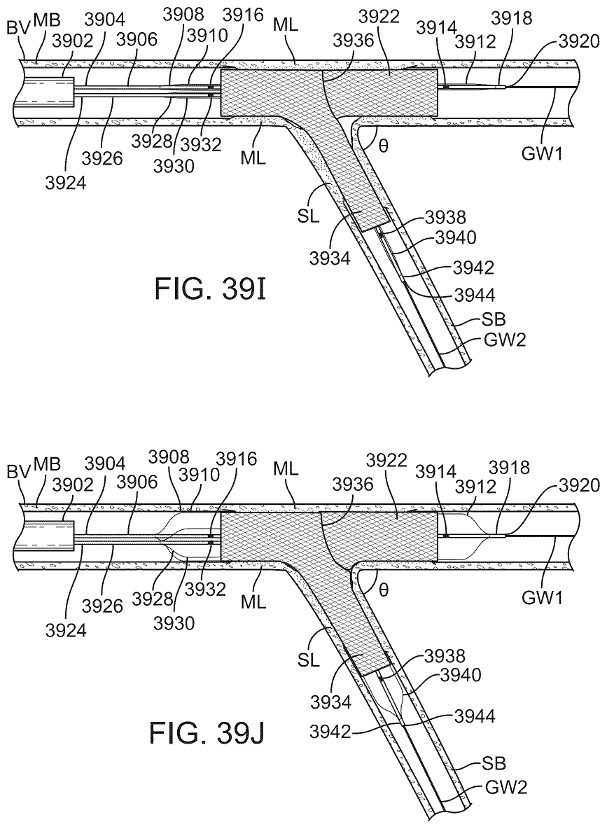

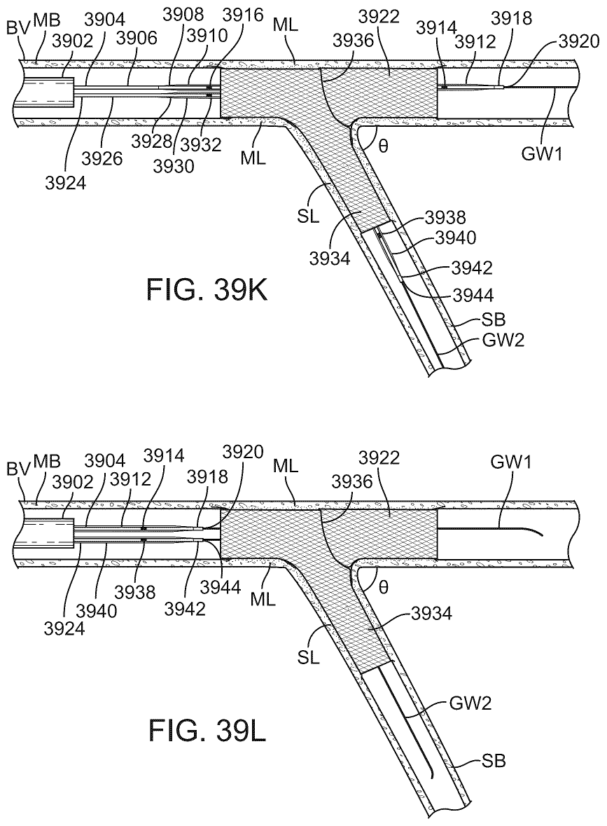

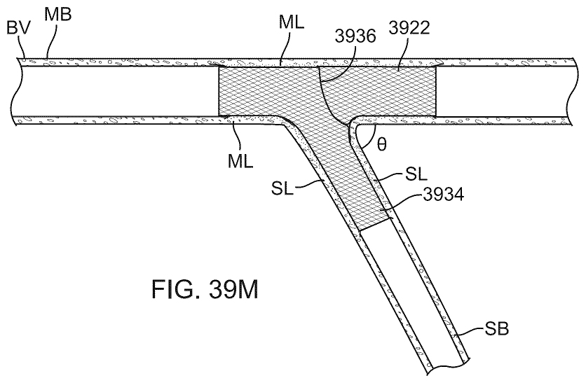

FIGS. 39A-39M more clearly illustrate another example of a method for treating a bifurcated vessel. This method is similar to that previously disclosed, with the major difference being that the distal-most catheter is used to treat the main branch vessel, and the proximal-most catheter is used to treat the side branch vessel. In the previous example, the distal-most catheter is used to treat the side branch vessel and the proximal-most catheter is used to treat the main branch.

In FIG. 39A, the bifurcated vessel BV includes a side branch vessel SB and a main branch vessel MB. The main branch has a main branch lesion ML, and the side branch has a side branch lesion SL. The angle between the side branch and the main branch is referred to as the bifurcation angle, and is indicated by θ. When the bifurcation angle θ is less than about 60 to 70 degrees, the distal-most stent of the system can be effectively positioned in the side branch. However, when the bifurcation angle is greater than or equal to about 60 to 70 degrees, it becomes more challenging to position the distal-most stent in the side branch. Moreover, when the distal stent is retracted proximally toward the stent having the side hole (discussed below), the catheter shaft may bind against the side hole resulting in damage to the catheter shaft and/or stent. Therefore, in some examples, when the bifurcation angle is less than about 60 to 70 degrees, the distal-most stent is for example positioned in the side branch and the proximal-most stent is advanced into the main branch. When the bifurcation angle is greater than or equal to about 60 to 70 degrees, the distal-most stent is positioned in the main branch and the other stent is positioned partially in the main branch and partially in the side branch. This is not intended to limit the use of the catheter system, and either stent may be placed in either side branch or main branch depending on operator preference. In FIG. 39B, a guide catheter 3902 is advanced distally into the vessel until it is adjacent the bifurcation and the lesions ML, SL. A first guidewire GW1 is advanced distally in the main branch MB until it is distal of the main branch lesion ML. A second guidewire GW2 is also advanced distally until it enters the side branch SB and it is distal of the side branch lesion SL.

In FIG. 39C, a treatment system having a first catheter 3904 and a second catheter 3924 (best seen in FIG. 39D) are advanced distally through the guide catheter 3902 toward the bifurcation. The two catheters 3904, 3924 may be advanced independently of one another, or the two catheters 3904, 3924 may for example be advanced simultaneously. The first catheter 3904 includes an elongate shaft 3906 with a radially expandable balloon 3908 on a distal portion of the elongate shaft 3906. A stent 3922 is disposed over the balloon 3908. The length of the stent 3922 may substantially match the working length of the balloon 3908, or the length of the stent 3922 may be less than the working length of the balloon 3908 such that a proximal portion 3910 and a distal portion 3912 of the balloon 3908 remains unconstrained by the stent 3922. A proximal radiopaque marker 3916 and a distal radiopaque marker 3914 may be used to help determine the proximal and distal ends of the balloon 3908 as well as the proximal and distal ends of the stent 3922. A soft durometer polymer tip may be used on the distal portion of the catheter shaft 3906 so as to prevent trauma to the vessel during delivery, and the catheter shaft 3906 has a distal guidewire port 3920 to allow a guidewire GW1 to enter or exit a guidewire lumen (not shown) in the catheter shaft 3906. The first catheter 3904 may be a rapid-exchange catheter or it may be an over-the-wire catheter. The second catheter 3924 (best seen in FIG. 39D) includes an elongate shaft 3926 having a radially expandable balloon 3928 on a distal portion thereof. A second stent 3934 is disposed over the second balloon 3928. The stent length may substantially match the working length of the balloon 3928, or it may be less. In this example, the length of stent 3934 is less than the working length of balloon 3928; thus, a proximal portion 3930 and a distal portion 3940 of the balloon 3938 remain unconstrained by the stent 3934. A portion of the first elongate shaft 3906 is disposed under a proximal portion of the second stent 3934, and the stent 3934 also has a side hole 3936 so that the first elongate shaft 3906 may exit therefrom. The first elongate shaft 3906 may slide under the stent 3934 relative to the second elongate shaft 3926; thus, a proximal portion 3910 of balloon 3908 is also disposed under stent 3934. When balloon 3908 is expanded, a proximal portion of stent 3934 will also be expanded. The second elongate shaft 3926 also includes a proximal radiopaque marker 3932 and a distal radiopaque marker 3938 that help identify the proximal and distal ends of the balloon 3928 and the proximal and distal ends of the stent 3934. The second catheter 3924 also has a soft durometer polymer tip 3942 that helps minimize trauma to the vessel during delivery, and a distal guidewire port 3944 allows a guidewire to be inserted or to exit from a guidewire lumen (not shown) in the elongate shaft 3926. The second catheter 3924 may be an over-the-wire catheter or it may be rapid exchange. The first stent 3922 and balloon 3908 are distal to the second stent 3939 and second balloon 3928.

In FIG. 39D, the bifurcation angle θ is greater than about 60 to 70 degrees. Both catheters 3904, 3924 are further advanced distally toward the bifurcation until the first stent 3922 is distal to the main branch lesion ML, and the second stent 3934 is partially disposed in the side branch SB adjacent the side branch lesion SL, and the stent 3934 is also disposed in the main branch MB adjacent the main branch lesion ML. The side hole 3936 also faces generally in the direction of the main branch vessel MB. Advancement of both catheters 3904, 3924 is for example performed simultaneously, although they could also be advanced independently of one another. The operator will feel resistance against further advancement of the catheters 3904, 3924 because as the catheters are advanced further distally, the two catheter shafts 3906, 3926 will spread apart relative to one another as they are forced against the carina of the bifurcation. However, a portion of the first elongate shaft 3906 is disposed under a portion of the second stent 3934, therefore the two shafts 3906, 3926 can only spread apart so far. Thus, when an operator feels resistance against further advancement of the catheter shafts, the operator knows that both catheters 3904, 3924 and their associated stents and balloons are properly positioned relative to the bifurcation.

In FIG. 39E the first catheter 3904 is retracted proximally relative to the second catheter 3924 so a proximal portion 3910 of balloon 3908 is disposed under stent 3934. Stent 3934 has a distal portion crimped to balloon 3928 so that it will not be ejected during delivery, and a proximal portion is partially crimped or uncrimped over balloon 3928 to allow shaft 3906 to slidably pass thereunder. Stent crimping is described in greater detail in U.S. patent applications previously incorporated by reference above. Because a portion of the first catheter shaft 3906 is disposed under a portion of the second stent 3934, the first shaft 3906 is slidably retracted into side hole 3936 and the first shaft 3906 is also slidably retracted under a portion of second stent 3934. The first shaft 3906 is proximally retracted until proximal radiopaque marker 3916 lines up with proximal radiopaque marker 3932 so that a proximal end of the first stent 3922 will be aligned with the side hole 3936 in the second stent 3934. An operator may feel resistance during retraction of the first elongate shaft 3906 relative to the second elongate shaft 3926 when the ends of the stents 3922, 3934 engage one another. The ends of the stents 3922, 3934 may butt up against one another, overlap with one another, interleave with one another, or combinations thereof. Additional details related to the engagement of the stents are disclosed in U.S. patent applications previously incorporated by reference above. Both stents 3922, 3934 are disposed adjacent their respective lesions SL, ML, and the side hole 3936 is in rough alignment with the main branch vessel MB.

In FIG. 39F, the balloon 3908 is radially expanded, often with contrast medium, saline, or a combination thereof thereby radially expanding the first stent 3922 into engagement with the main branch lesion ML and the walls of the main branch. A proximal portion of the second stent 3934 is also expanded into engagement with the main branch lesion ML and the walls of the main branch, while a distal portion of the second stent 3934 remains unexpanded in the side branch SB. The first stent 3922 and the proximal portion of the second stent 3934 are radially expanded simultaneously. The inner surfaces of both stents form a smooth lumen for blood flow through the main branch. Since a portion of balloon 3908 also passes through side hole 3936, expansion of balloon 3908 also partially expands the side hole 3936 and also aligns the side hole 3936 with the main branch lumen.

In FIG. 39G the balloon 3908 is contracted, and then in FIG. 39H the other balloon 3928 is radially expanded, with contrast medium, saline, or a combination thereof, thereby further radially expanding the second stent 3934. Expansion of balloon 3928 expands a distal portion of stent 3934 into engagement with the side branch vessel wall and side branch lesion SL. The proximal portion of stent 3934 and side hole 3936 may also be further expanded and aligned with the first stent 3922. The side hole 3936 is also further aligned with the lumen of the main branch.

Referring now to FIG. 39I, balloon 3928 is contracted and then both balloons are simultaneously inflated in a “kissing balloon” technique as seen in FIG. 39J. Both balloons 3908, 3928 are inflated with contrast medium, saline, or combinations thereof until they engage one another and are fully expanded in the main branch MB and side branch SB. The kissing balloon technique ensures that both stents 3922, 3934 are fully expanded and in full apposition with their respective vessel wall and lesion. Additionally, the kissing balloon technique lines up the proximal end of the first stent 3922 with the side hole 3936 in the second stent 3934, thereby ensuring that continuous and smooth scaffolding from the main branch MB into the side branch SB. Alignment of the two stents is disclosed in greater detail in U.S. patent applications previously incorporated by reference above. Also, the kissing balloons technique ensures that the side hole does not block the main branch or disrupting blood flow across the bifurcation.

In FIG. 39K, both balloons 3908, 3928 are contracted, and in FIG. 39L both catheters 3904, 3924 are retracted proximally. The catheters may be retracted simultaneously or independently of one another. The first catheter 3904 is retracted through both stents 3922, 3934 and also passes through the side hole 3936. The second catheter 3924 is retracted through the second stent 3934. In FIG. 39M, both catheters 3904, 3924 have been removed, as well as the guide catheter 3802 and both guidewires GW1, GW2. Stents 3922, 3934 remain implanted in at the bifurcation. Optionally, the stents or balloons may contain therapeutic agents such as those previously discussed, and these may elute out into the lesion at a controlled rate in order to help prevent restenosis.

Any of the methods described above may use any of the stents disclosed herein in any of the system configurations described. Additionally, any of the features previously described above may also be used. Therefore, one of skill in the art will appreciate that any number of combinations may be made. For example, catheter systems may have any combination of rapid exchange or over-the-wire configurations, with any of the stents disclosed herein, with or without a therapeutic agent on a stent or a balloon, and with or without any of the hollow exchange port, capture tube, removable capture tube, or snap fittings described above.

### Stents:

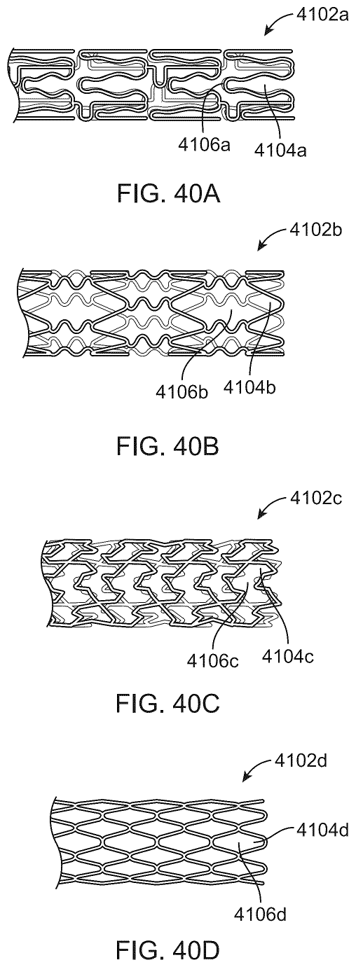

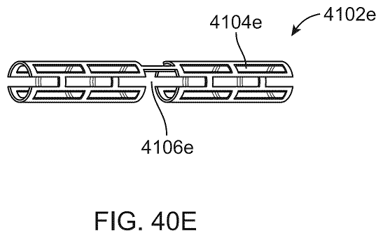

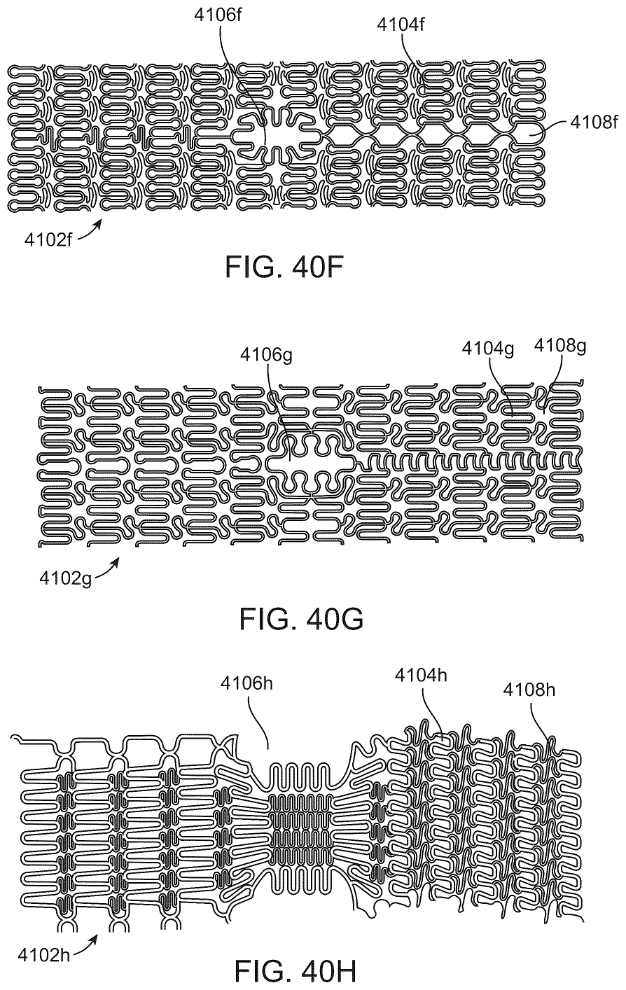

The catheter systems and methods described above may use a commercially available stent for either the proximal or distal stent in the system. When a commercially available stent is used for the distal stent, it need only be crimped to the distal balloon catheter. When the commercially available stent is used for the proximal stent it may be partially crimped to the proximal balloon such that a portion of a second catheter shaft is slidably disposed under the stent and a portion of the second catheter shaft slidably passes through a side hole in the stent. The stent is crimped to the proximal balloon so that it is not displaced from the balloon during delivery, and also so the second catheter shaft can slide thereunder. FIGS. 40A-40E illustrate several examples of commercially available stents that may be used in catheter system configurations and methods described above, either as is, or with slight modification. For example, FIG. 40A illustrates the Abbott Vascular Xience® drug eluting stent 4102a. A portion of a catheter shaft may be disposed under the stent 4102a through its central channel and the catheter may exit a side hole in the stent 4102a. A side hole may be the gap 4104a created between adjacent struts in a cell, or the gap 4106a between axially adjacent cells. FIG. 40B illustrates the Cordis Cypher® stent 4102b. Again, a portion of a catheter shaft may be disposed under the stent 4102b through its central channel and the catheter may exit a side hole in the stent 4102b. A side hole may be the gap 4104b created between adjacent struts in a cell, or the gap 4106b between axially adjacent cells. FIG. 40C illustrates the Boston Scientific Taxus® Liberte® stent 4102c. A portion of a catheter shaft may be disposed under the stent 4102c through its central channel and the catheter may exit a side hole in the stent 4102c. A side hole may be the gap 4104c created between adjacent struts in a cell, or the gap 4106c between axially adjacent cells. FIG. 40D illustrates the Medtronic Endeavor® stent 4102d. A portion of a catheter shaft may be disposed under the stent 4102d through its central channel and the catheter may exit a side hole in the stent 4102d. A side hole may be the gap 4104d created between adjacent struts in a cell, or the gap 4106d between axially adjacent cells. FIG. 40E illustrates a Palmaz-Schatz® stent 4102e. A portion of a catheter shaft may be disposed under the stent 4102e through its central channel and the catheter may exit a side hole in the stent 4102e. A side hole may be the gap 4104e created between adjacent struts in a cell, or the gap 4106e between axially adjacent segments.

Other stents have been designed with side holes that are specifically intended to treat bifurcations. These stents may also be used with the systems and method disclosed herein. For example, FIGS. 40E-40H illustrate several examples of stents from Boston Scientific and are disclosed in detail in U.S. Pat. No. 7,678,142. FIG. 40F shows a stent 4102f after it has been unrolled and flattened having a side hole 4106f. FIG. 40F illustrates a stent geometry (unrolled, plan view) where the struts create a side hole 4106f that allows access to a side branch, and that can accommodate a catheter shaft as described herein. The side hole 4106f may be formed by the spaces 4104f, 4108f between struts. FIG. 40G illustrates another stent geometry (unrolled, plan view) of stent 4102g having a side hole 4106g. Alternatively, the side hole 4106g may be formed by the spaces 4104g, 4108g between struts or axial connectors. FIG. 40H illustrates still another stent geometry (unrolled, plan view) of stent 4102h having a side hole 4106h. The side hole 4106h may also be formed by the space between struts 4104h or axial connectors 4108h. In any of these examples, a catheter shaft may be slidably disposed under a portion of the stent, and the catheter shaft may exit the side hole. Additionally, any of the stents or balloons disclosed herein may carry a therapeutic agent such as those described above for local drug delivery. Also, while the stents disclosed herein are for example balloon expandable, one of skill in the art will appreciate that self-expanding and hybrid balloon expandable/self-expanding stents may also be used.

### Stent Alignment:

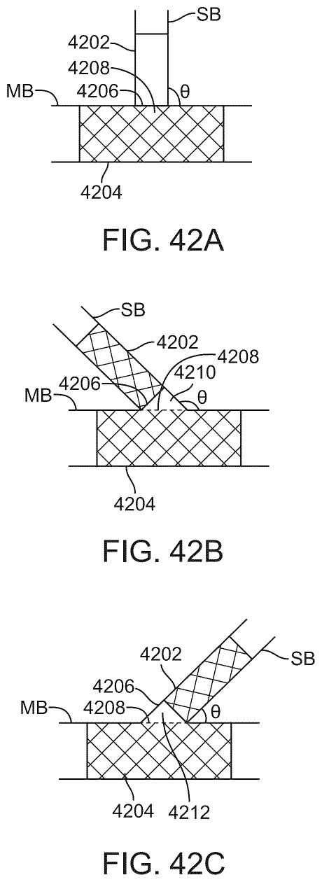

FIGS. 42A-42C illustrate various ways a side branch stent can line up with a main branch stent. In FIG. 42A, the side branch SB is substantially perpendicular to the main branch MB; therefore, the bifurcation angle θ is about 90 degrees. In this situation, the proximal end 4206 of the side branch stent 4202 will be substantially flush with the side hole 4208 in the main branch stent 4204 (assuming proper deployment of both stents). This is desirable since there are no gaps and hence no unscaffolded regions between the two stents 4202, 4204. However, when the bifurcation angle θ increases (FIG. 42B) or decreases (FIG. 42C), a portion of the side branch will remain unstented. For example, in FIG. 42B the bifurcation angle increases, and because of the right cylindrical shape of the stent, in which the end is perpendicular to the sidewalls of the stent, a gap 4210 exits between the proximal end 4206 of the side branch stent 4202 and the side hole 4208 of the main branch stent 4204. Similarly, in FIG. 42C, when the bifurcation angle decreases, there is also a gap 4212 between the proximal end 4206 of stent 4202 and the side hole 4208 of stent 4204. FIG. 42C is typical of human anatomy, therefore the gap 4212 often is upstream of the bifurcation. Gaps are undesirable since they are unscaffolded and recoil, and restenosis may occur in this region. Additionally, in the case where a stent is used for drug elution, the gap region may not receive any of the drug.

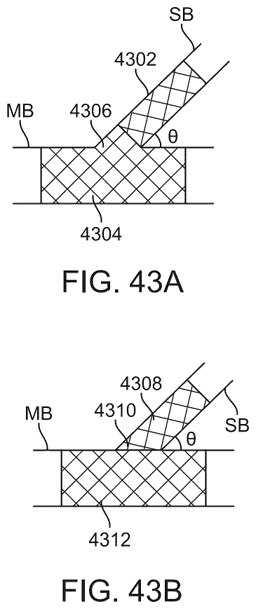

One possible solution for ensuring that the gap between a side branch stent and a main branch stent is eliminated or reduced is shown in FIG. 43A. The side branch stent 4302 is a right cylindrical stent. The main branch stent 4304 has a side hole 4306 with struts that expand outwardly into the gap region, thereby ensuring continuous scaffolding. An alternative solution in FIG. 43B is to fabricate the proximal end 4310 of the side branch stent 4308 with its proximal end non-perpendicular to the central axis of the stent so that the proximal end of the side branch stent lines up with the side hole in the main branch stent 4312. Even using the geometries illustrated in FIG. 43A-43B still requires careful alignment of the side branch stent with the main branch side hole. Therefore, it would be desirable to provide a stent geometry that facilitates alignment.

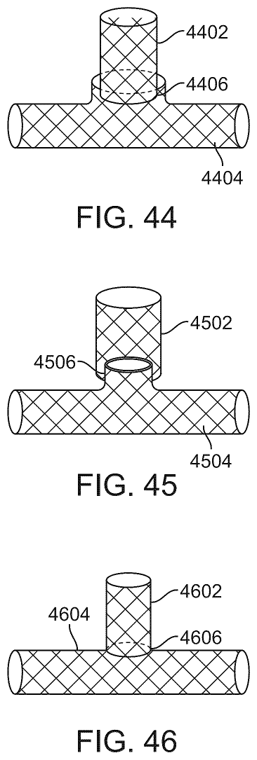

The ends of the side branch stent and the main branch stent may intersect in several different ways thereby providing continuous and uniform coverage of the bifurcation. For example, in FIG. 44, a portion 4406 of side branch stent 4402 may be disposed inside main branch stent 4404. FIG. 45 shows a portion 4506 of the main branch stent 4504 disposed inside the side branch stent 4502. Neither situation in FIG. 44 or 45 is ideal as overlapping of stents may result in metal rubbing on metal as well as possibly disrupting blood flow or causing stagnation points. A more desirable interface between stents is shown in FIG. 46 where the end of the side branch stent 4602 butts up against the side hole in main branch stent 4604. The interface region 4606 is desirable since it provides continuous scaffolding of the vessel without gaps between ends of the stents. However, depending on the stent geometry, gaps may still exist between stents. Therefore, in some examples, the ends of the stents will interleave or interdigitate with one another.

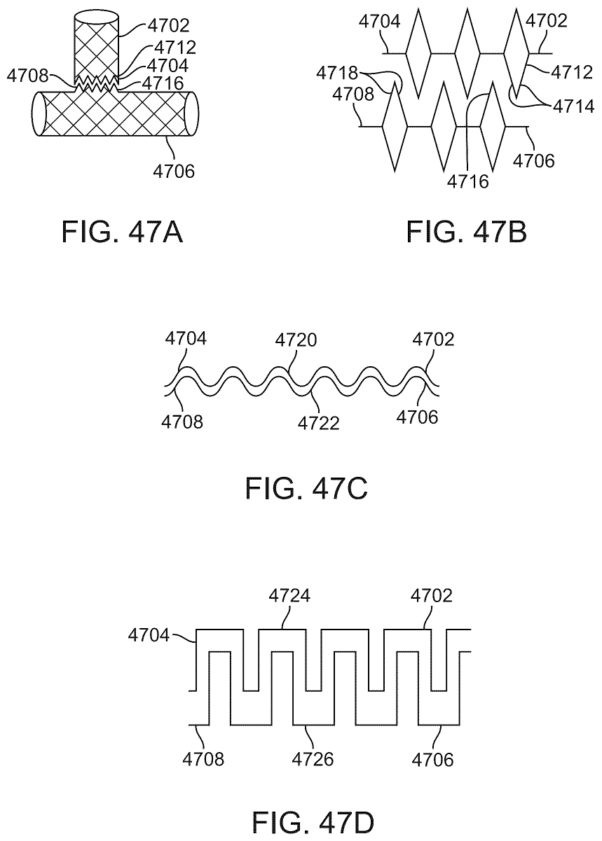

FIGS. 47A-47D illustrate several examples where the ends of the side branch stent and the side hole of the main branch stent interleave with one another or interdigitate. For example, in FIG. 47A, a proximal end 4704 of side branch stent 4702 has a series of axially extending elements or fingers 4712 which interdigitate or interleave with the laterally extending elements or fingers 4716 that extend laterally from the side hole 4708 of main branch stent 4706. FIG. 47B illustrates an example of interdigitating axial and lateral elements. A proximal end 4704 of side branch stent 4702 has a plurality of axially extending elements 4712. The axially extending elements 4712 are formed from a plurality of interconnected stent struts 4714, in this case forming a triangular shape. Similarly, the side hole 4708 of the main branch stent 4706 has a plurality of laterally extending elements 4716 that are formed from a plurality of interconnected stent struts 4718. In this case the laterally extending elements 4716 are formed into a triangular shape. Thus, the apex of one triangular shaped element fits in between adjacent elements on the adjacent stent. Or alternatively, the peaks fit in the valleys, and the valleys receive the peaks.

FIG. 47C illustrates still another example of interleaving or interdigitating elements. The proximal end 4704 of the side branch stent 4702 includes a strut 4720 formed into a series of peaks and valleys. Similarly, the side hole 4708 of the main branch stent 4706 will also have a strut 4722 that has been formed into a series of peaks and valleys. Therefore, the peaks of the side branch stent 4702 will fit into the valleys of the adjacent main branch stent side hole, and similarly the valleys of the side branch stent 4702 receive the peaks of the side hole. FIG. 47D illustrates yet another example of interleaving or interdigitation of stent ends. The proximal end 4704 of side branch stent 4702 includes a strut 4724 formed into a series of rectangular peaks and valleys. The side hole 4708 of the main branch stent 4706 also has a strut 4726 formed into a series of rectangular peaks and valleys. The peaks and valleys interleave and interdigitate with one another.

### Balloon Configurations:

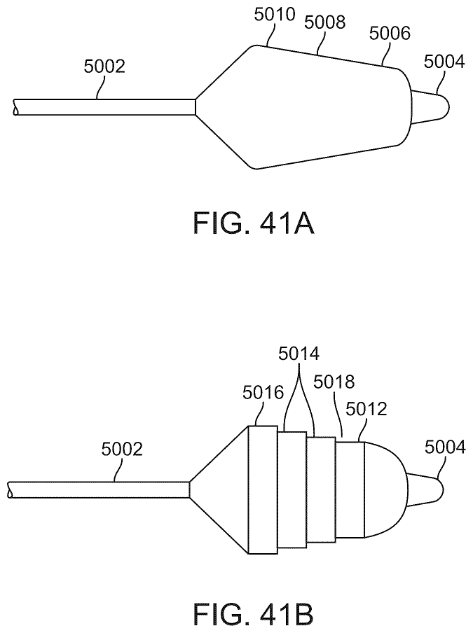

The balloons used to radially expand the stents described herein may be cylindrical balloons having a constant diameter along the working length, or diameter may vary. When stenting a tapered vessel, it may be beneficial to use a balloon which has a variable diameter balloon that more closely matches the vessel anatomy. For example, in FIG. 41A, a tapered balloon 5006 is attached to the distal portion of shaft 5002. A soft durometer tip 5004 prevents vessel trauma during delivery. The balloon 5006 is tapered such that a proximal portion 5010 of the balloon 5006 has a larger diameter than a distal portion. Any taper may be used. FIG. 41B illustrates another example of a balloon 5012 having a plurality of stepped regions 5014. The stepped regions 5014 may be incremented in any amount, and in some examples, a proximal portion 5016 of the balloon 5012 has a larger diameter than a distal portion 5018. Any of these examples, or combinations thereof, may be used in the systems and methods described herein to treat a bifurcation. Use of a tapered or stepped balloon allows a stent to be expanded to more closely match the vessel walls, where a proximal portion of the expanded stent has a larger diameter than a distal portion of the stent.

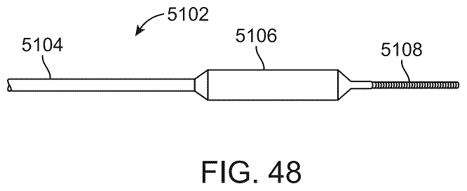

In addition to using catheters having rapid exchange or over-the-wire guidewire lumens, and tapered or stepped balloons, the balloon catheters may not always employ a guidewire lumen. Instead, a fixed wire may be attached to a distal end of the catheter. For example, FIG. 48 illustrates an example of a fixed wire catheter 5102 having a balloon 5106 attached to a distal portion of the shaft 5104. A section of guidewire 5108 is fixedly attached to the distal end of the catheter 5102 and this fixed wire helps the catheter 5102 track through the vessels. The fixed wire may have any number of shapes including straight, curved, J-tip, etc. This example may be used with any of the systems and methods disclosed herein, and it may or may not have a stent crimped to the balloon. The fixed wire catheter may 5102 be used in the main branch, or more for example it may be used in the side branch.

### Twist Resolution:

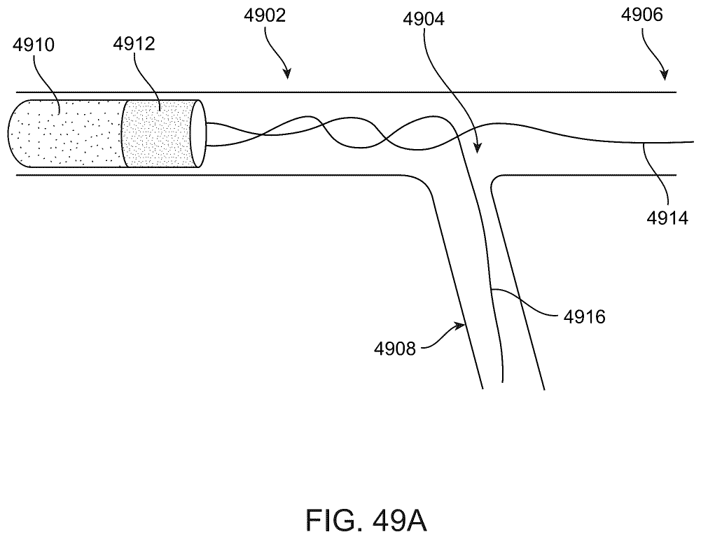

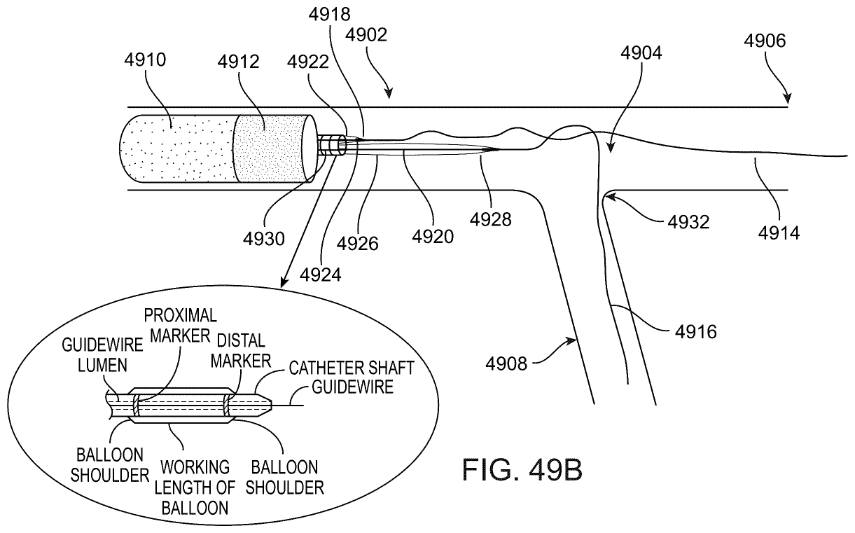

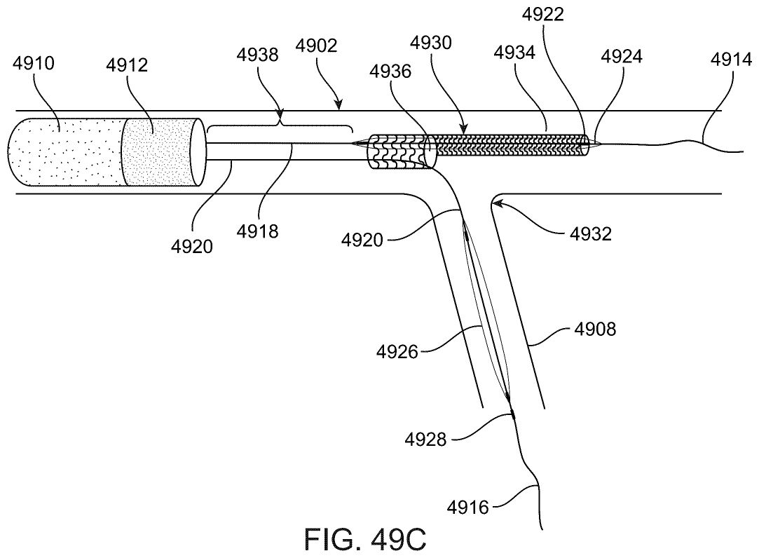

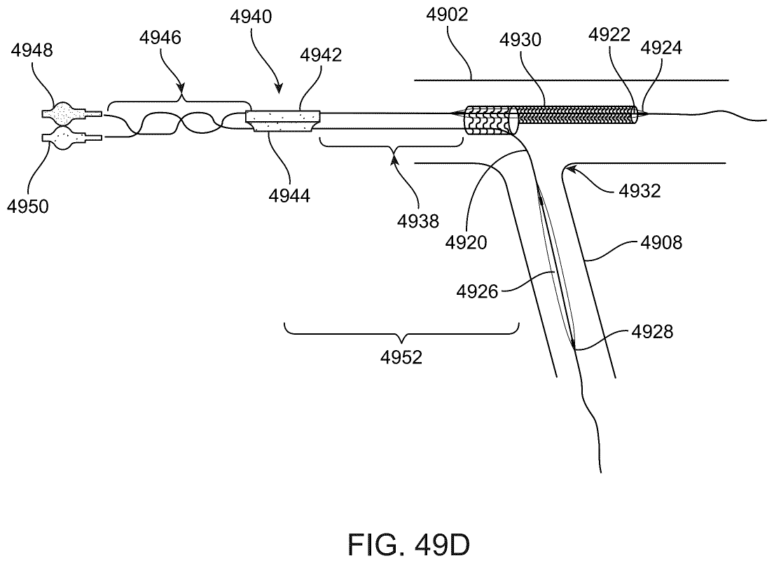

FIGS. 49A-49D include schematic views illustrating aspects of twist resolution techniques of dual catheter systems described herein. In some examples, twisting is also referred to in the art as “wire crossing”. In FIG. 49A, a blood vessel 4902 splits (or bifurcates) into a main branch 4906 and a side branch 4908 at a bifurcation 4904. In some examples, main branch is also referred to as “mother vessel” and the side branch is referred to as “daughter vessel.” The tip 4912 of a guide catheter 4910 is visible in the views. The guide catheter 4910 guides two guidewires, a main branch guidewire 4914 and a side branch guidewire 4916, through the blood vessel 4902. Beyond (distally) of the bifurcation 4904, the guidewires 4914 and 4916 pass into the main branch 4906 and the side branch 4908, respectively. However, when the guidewires 4914 and 4916 are advanced through the blood vessel 4902, the guidewires 4914, 4916 can (and often do) twist around one another and can become entangled, as shown in FIG. 49A. Guidewire twisting and entanglement can present a significant problem and misdirect and prevent full deployment of stents and other devices, for example.

In FIG. 49B, a main branch catheter 4918 is deployed over the main branch guidewire 4914. The main branch catheter 4918 may include a main branch balloon 4922 and a main branch radiopaque marker 4924. A side branch catheter 4920 is deployed over the side branch guidewire 4916. The side branch catheter 4920 may include a side branch balloon 4926 and a side branch radiopaque marker 4928. One or both of the main and side branch catheters 4918 and 4920 may carry a stent 4930 for deployment at the bifurcation 4904 in accordance with any one or more of the stents, catheters, stent deployment and/or bifurcation treatment procedures described above. Various arrangements of the components illustrated in FIGS. 49A-49B are thus possible.

For example, the arrangements and positions of the radiopaque markers in FIGS. 49A-49D are shown merely by way of example in schematic outline only. Other arrangements and positions of the radiopaque markers are possible. An example arrangement is shown in the inset view of FIG. 49B. In some examples, a radiopaque marker is positioned under a balloon, aligned with an edge of the working length of the balloon and the transition to the shoulder of the balloon. The edges of the stent are also aligned with the same position on the balloon and position of the marker.

As described above with the guidewires, when the shafts of the catheters 4918 and 4920 are advanced through the blood vessel 4902, the catheter shafts often twist around one another and can become entangled. Catheter shaft twisting and entanglement can also present a significant problem and misdirect and prevent full deployment of stents and other devices, for example. Also, in some examples, because the catheters are delivered over the guidewires, the catheters follow the twisted guidewires and so the catheters end up being twisted, in addition to their own twisting/entanglement independent of the guidewires.

However, in some examples of the dual catheter systems described herein, when the twisted regions of the guidewires 4914 and 4916 and/or the shafts of the catheters 4918 and 4920 are pushed against the carina 4932 of the bifurcation 4904, the twisting of the guidewires and/or catheters is pushed back proximally (i.e., towards the guide catheter 4910) with the result that the guidewires and catheter shafts distal of the carina 4932 become untwisted and extend straight in the main branch 4906 and the side branch 4908, as shown. In some examples, advancement of the wires or catheters against the carina at a bifurcation causes or generates a force, or reactive force, to separate the wires and/or catheters away from one another resulting in the untwisting.

In some examples, when the two catheters 4918 and 4920 are advanced over installed guidewires 4914 and 4916 distally against the carina, the twists in the catheters 4918 and 4920 get pushed back proximally. In some examples, once the guidewires 4914 and 4918 are delivered, they are not moved forward or backward. The action of the catheters 4918 and 4920 moving against the carina cause the untwisting and pushing back of the twists. This catheter twist resolution can mitigate some of the entanglement issues discussed above.

In some examples, reference is made to guidewires or catheters being advanced “against” a carina at a bifurcation to push twists in the guidewires or catheters back proximally. Unless the context is clear or implies otherwise, the use of the term “against” the carina is intended to include situations in which an untwisting guidewire and/or catheter (as the case may be) bears “directly” against a carina (i.e., while bare or uncovered for example), and also situations in which the untwisting guidewire or catheter bears “indirectly” against a carina, for example while covered by another element such as another catheter, a medical instrument, or is passing through a device, such as a stent located at the carina for example.

In FIG. 49C, the stent 4930 is advanced to arrive at the site of the bifurcation 4904. The stent 4930 can be deployed (for example, expanded) using any one of the stent deployment and/or balloon inflation techniques described further above. In some examples, the stent 4930 has a first or main branch portion 4934 and a side hole 4936. The main branch portion 4934 is carried on the main branch catheter 4918 and can be expanded by the main branch balloon 4922 (as described further above for example) to deploy, crimp, and lock the stent 4930 in place at the site of the bifurcation 4904.

In some examples, the side branch catheter 4920 passes through the side hole 4936 of the stent 4930, as shown. In the illustrated view, the side branch catheter 4920 (carrying the side branch balloon 4926 and the side branch radiopaque marker 4928) has been advanced distally past the carina 4932 of the bifurcation 4904 to enter untwisted (or at least unentangled) and in straight manner into the side branch 4908. The side branch balloon 4926 may be inflated to expand and deploy a side branch stent (not shown in the interest of clarity) using one or more of the side branch stent deployment techniques described further above, for example.

Similarly, the main branch catheter 4918 (carrying the main branch balloon 4922 and the main branch radiopaque marker 4924) has been advanced distally past the carina 4932 of the bifurcation 4904 to enter untwisted (or at least unentangled) and in straight manner into the main branch 4906. The presence of the deployed (expanded) stent 4930 may serve to lock the stent 4930 rotationally relative to the carina 4932 and serve as a guide for the catheters 4918 and 4920. The rotationally locked stent 4930 may serve to reinforce the carina 4932 and/or assist in untwisting the catheters 4918 and 4920 and pushing twists in the catheters 4918 and 4920 back proximally. An untwisted zone may be created, as shown in the untwisted region 4938 in FIG. 49C for example. In some examples, the action of the catheters 4918 and 4920 moving against the carina initially cause creation of a twisted zone adjacent the carina. Further action of the catheters 4918 and 4920 moving against the carina cause the twisted zone to clear and push back the twists.

As the catheters advance, twists and entanglements of the catheters are pushed back proximally (safely towards the catheter operator, and away from the treatment site), and into or past the guide catheter 4910 in some examples. The pushing back of twists and entanglements is accomplished or at least assisted in some examples by the resistance offered by the carina at a treated bifurcation to part twisted guidewires and/or catheters. The pushing back of twists and entanglements is accomplished or at least assisted in some examples by a stent deployed at a treated bifurcation acting as a “locked” guide serving to untwist entangled guidewires and/or advancing catheters. The pushing back of twists and entanglements in guidewires and/or advancing catheters is accomplished or at least assisted in some examples by the carina and stent acting in concert together as when used for example in the dual mode catheter systems and methods described herein. As described above with reference to FIG. 38D, an operator may feel resistance against further advancement of the catheters because as the catheters are advanced further distally, the two catheter shafts will spread apart (and untwist) relative to one another as they are forced against the carina of the bifurcation.

With reference to FIG. 49D, the proximal pushing or passing back of twists and entanglements can be assisted by the employment of a slider or “pocket” 4940. In some examples, the pocket 4940 includes or is constituted by a hollow exchange port tube, or a capture tube, or a removable capture tube, or a zipper or snap fitting as described further above. An example pocket 4940 illustrated in FIG. 49D has two channels, for example tubular or hollow channels, to accommodate and guide in sliding fashion the shafts of the main branch catheter 4918 and the side branch catheter 4920, respectively. Other arrangements and configurations of the pocket 4940 are possible. In the illustrated view, the pocket 4940 includes a tubular main branch catheter channel 4942 and a side branch catheter channel 4944. The pocket 4940 serves to pass twists and entanglements proximally and safely into a twist collection region 4946 nearer an operator handling catheter connectors 4948 and 4950, for example. In some examples, when the twists unwrap at the distal end of the dual-catheter system, there is little to no “whiplash” experienced at the proximal end, for example nearer an operator or away from the treatment site. In other words, as the distal ends of the wires and/or catheters untwist to release twists and wire crosses, the proximal end of the wires and/or catheters rotate smoothly and consistently without a jerky or start/stop rotation.

In some examples, the pocket 4940 is interposed between the bifurcation and the guide catheter tip, in use. In some examples, the pocket 4940 is positioned in use on the catheter shafts at a pocket-to-bifurcation distance 4952 in the range 30-100 millimeters (mm). In some examples, the pocket 4940 is positioned in use on the catheter shafts at a pocket-to-bifurcation distance 4952 in the range 30-50 mm.

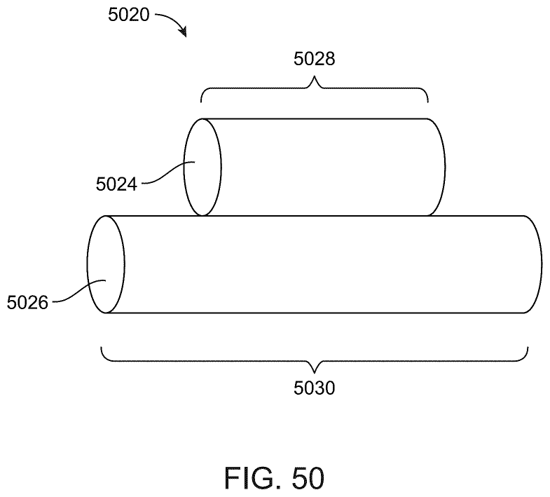

An enlarged pictorial view of an example pocket 5020 appears in FIG. 50. The pocket 5020 includes a first catheter shaft channel 5024 and a second catheter shaft channel 5026. A length 5028 of the first catheter shaft channel 5024 may be the same as, shorter, or longer than a length 5030 of the second catheter shaft channel 5026. A diameter or cross-sectional area of the first catheter shaft channel 5024 may be the same as, smaller, or greater, than a diameter or cross sectional area of the second catheter shaft channel 5026.

The length 5028 of the first catheter shaft channel 5024 may be in a range 5-60 mm, or in a range 10-40 mm, or in a range 20-40 mm, or be 36 mm. The length 5030 of the second catheter shaft channel 5026 may be in a range 5-60 mm, or in a range 10-40 mm, or in a range 20-40 mm, or be 36 mm. The first catheter shaft channel 5024 may have an outer diameter in a range 0.040-0.050 mm, and an internal diameter in the range 0.035-0.045 mm. The second catheter shaft channel 5026 may have an outer diameter in a range 0.030-0.040 mm, and an internal diameter in the range 0.025-0.035 mm.

The pocket 5020 (or at least one of the catheter shaft channels 5024 and 5026) may be coextruded with a shaft of the main branch catheter or the side branch catheter, or it may be bonded or otherwise attached thereto using techniques known to those skilled in the art. The catheter shaft channels are sized to slidably receive a portion of a catheter shaft passing therethrough. In some examples, an elongate slot or slots (not shown) may extend along the entire length of the pocket 5020 and be sized such that the pocket 5020 may be snapped onto one or both catheter shafts ready for use.

Some examples of dual catheter systems include asymmetric profiles to assist with twist resolution. For example, a catheter shaft may include a non-circular portion that helps with twist resolution, and/or in proximally pushing or passing back twist and entanglements as discussed above. To this end, a catheter shaft (such as the main branch catheter and/or the side branch catheter) may have an oval cross section, at least an oval cross section in a length of the shaft close to or traversing a treated bifurcation, or a pocket, for example. Other cross-sectional shapes are possible and may be beneficial in that they reduce the overall profile of the dual catheter system. For example, reference in this regard is made to FIG. 35 hereof which shows a pear-shaped overall profile that may be adopted for dual catheter profiles to assist in twist resolution techniques. Other overall profiles for dual catheters, or a pocket, or a pocket channel are possible, for example a figure of eight configuration. A cross section of a catheter shaft, pocket, or pocket channel having a cross-sectional transverse or lateral dimension being greater in one direction than another transverse or lateral dimension in another direction (for example, orthogonal thereto) may serve to impart anti-twist resistance by dint of the non-circular configuration presented by such a profile. An asymmetric cross-sectional profile of a catheter shaft may present a larger bearing surface area against a carina, for example, to impart a larger “anti-twist” torque or push-back force than a catheter shaft of circular cross section, for example. In some examples, a pocket 4940 and/or shaft 5002 may include a cross-sectional profile similar to that of the snap fitting 1324 disclosed in FIG. 13C, for example. In other words, one pocket channel may be larger in diameter or sectional area than another.

Some examples also relate more generally to untwisting wires or cables at the site of a bifurcation in broader applications, such as at a bifurcation in a tunnel in underground mining or boring, or at a bifurcation in downhole logging applications, for example. Disclosed twist resolution techniques may also be used at aboveground construction sites, for example, where twisting of wires and cables at bifurcations in guides or scaffolding structures may occur. Other twist resolution applications are possible in domestic or commercial IT cabling, such as fiber optic cable installation, for example. Other twist resolution applications are possible.

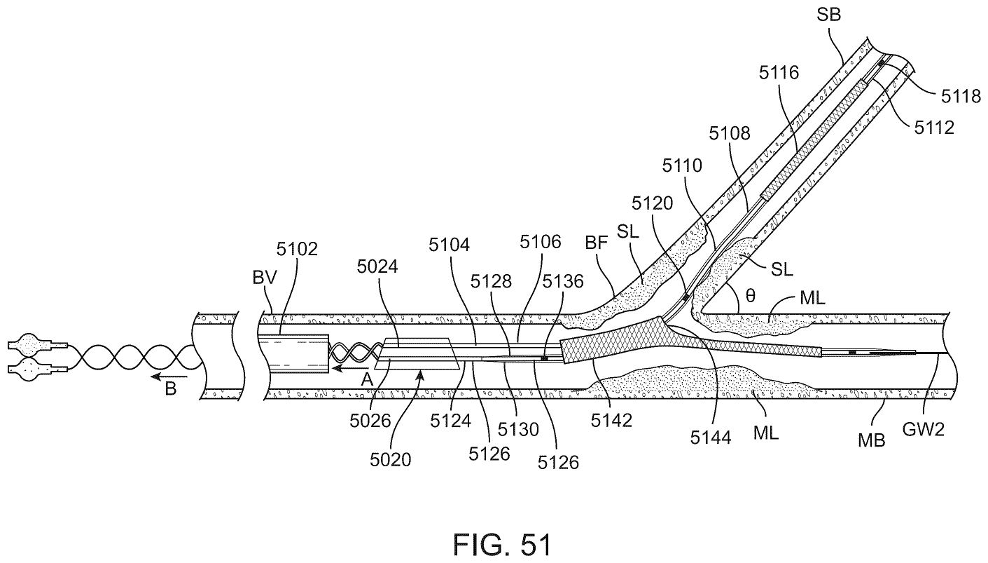

In medical device applications, FIG. 51 illustrates an example stent installed using example twist resolution techniques of a dual catheter system. In FIG. 51, both catheters 5104, 5124 are advanced distally toward a bifurcation BF until a first stent 5116 is distal to a side branch lesion SL and a second stent 5142 traverses a main branch lesion ML and a side hole 5144 is adjacent the ostium of the side branch SB. Advancement of both catheters 5104, 5124 is again performed simultaneously, although they could also be advanced independently of one another. The operator will feel resistance against further advancement of the catheters 5104, 5124 because as the catheters are advanced further distally, the two catheter shafts 5106, 5126 will spread apart relative to one another as they are forced against the carina of the bifurcation. However, a portion of the first elongate shaft 5106 is disposed under a portion of the second stent 5142; therefore, the two shafts 5106, 5126 can only spread apart so far. Thus, when an operator feels resistance against further advancement of the catheter shafts 5106, 5126, the operator knows that both catheters 5104, 5124 and their associated stents and balloons are properly positioned relative to the bifurcation. Further, while the catheter shafts 5106, 5126 are being advanced and pushed against the carina of the bifurcation, twists in the catheters are pushed back proximally in the direction of arrow A (i.e., towards the fixed wire catheter 5102) with the result that the guidewires and catheter shafts distal of the carina become untwisted and extend straight into the main branch MB and side branch SB, as shown. With the help of the pocket 5150 positioned on the catheter shafts, the pushing or passing back of the twists can extend into the region of arrow B and towards the catheter operator, as shown.

In subsequent operations, the first catheter 5104 is retracted proximally relative to the second catheter 5124. Because a portion of the first catheter shaft 5106 is disposed under a portion of the second stent 5142, the first shaft 5106 is slidably retracted into side hole 5144 and the first shaft 5106 and proximal portion 5110 of balloon 5108 are slidably retracted under a portion of second stent 5142. The first catheter shaft 5106 is proximally retracted until proximal radiopaque marker 5120 lines up with proximal radiopaque marker 5136 so that a proximal end of the first stent 5116 will be aligned with the side hole 5144 in the second stent 5142. An operator may feel resistance during retraction of the first elongate shaft 5106 relative to the second elongate shaft 5126 when the ends of the stents 5116, 5142 engage one another. Stent 5142 has a distal portion crimped to balloon 5128 to prevent ejection during delivery, and a proximal portion is partially crimped thereto or uncrimped to allow catheter 5104 to slide thereunder. Ultimately, both stents 5116, 5142 are disposed adjacent their respective lesions SL, ML, and the side hole 5144 is in rough alignment with the ostium to the side branch SB and the side branch stent 5116.

The balloon 5108 is radially expanded, often with contrast medium, saline, or a combination thereof thereby radially expanding the first stent 5116 into engagement with the side branch lesion SL and the walls of the side branch. A proximal portion 5110 and a distal portion 5112 of the balloon 5108 will also expand, thus a proximal portion of the second stent 5142 will also be radially expanded. Expansion of the stents occurs simultaneously. Since a portion of balloon 5108 also passes through side hole 5144, expansion of balloon 5108 also partially expands the side hole 5144 and also aligns the side hole 5144 with the ostium of the side branch.

In a subsequent operation, the balloon 5108 is contracted, and the other balloon 5128 is radially expanded, with contrast medium, saline, or a combination thereof, thereby further radially expanding the second stent 5142. Expansion of balloon 5128 expands the proximal portion of the stent 5142 into engagement with the main branch vessel wall and main branch lesion ML, and the distal portion of the stent 5142 is also radially expanded into the main branch vessel wall as well as the main branch lesion ML. The side hole 5144 is also further aligned with the ostium of the side branch SB. Kissing balloon techniques as described further above can be used to further align and engage the stents, followed by deflation of the balloons and retraction of the catheters, as described for example further above with reference to FIGS. 38A-38M.

Thus, in some examples, there is provided a system for treating a bifurcated vessel. An example system comprises a first delivery catheter, the first delivery catheter comprising a first elongate shaft with a proximal end and a distal end, a first expandable member adjacent the distal end of the first elongate shaft, and a first radially expandable stent disposed over the first expandable member, wherein the first radially expandable stent has a collapsed configuration and an expanded configuration, wherein in the collapsed configuration the first radially expandable stent is crimped on the first expandable member with the first expandable member in a delivery configuration for deployment in a main branch of a blood vessel, and in the expanded configuration the first radially expandable stent is expanded radially by expansion of the first expandable member from the delivery configuration so as to support a vessel wall; and a second delivery catheter comprising a second elongate shaft with a proximal end and a distal end, a second expandable member adjacent the distal end of the second elongate shaft, and a second radially expandable stent disposed over the second expandable member, wherein the second radially expandable stent has a collapsed configuration and an expanded configuration, wherein in the collapsed configuration the second radially expandable stent is crimped on the second expandable member with the second expandable member in a delivery configuration for deployment in a side branch of a blood vessel, and in the expanded configuration the second radially expandable stent is expanded radially by expansion of the first expandable member from the delivery configuration so as to support a vessel wall; and a twist resolution pocket disposed on the first delivery catheter or the second delivery catheter, the twist resolution pocket positioned and configured such that when the first delivery catheter and the second delivery catheter are advanced distally and pushed against a carina of a bifurcation located between the main branch and the side branch of the blood vessel, twisting in the first delivery catheter or the second delivery catheter is pushed back proximally through the first delivery catheter or the second delivery catheter and away from the bifurcation.

In some examples, the twist resolution pocket includes two catheter shaft channels including a first catheter shaft channel to receive the first elongate shaft of the first delivery catheter, and a second catheter shaft channel to receive the second elongate shaft of the second delivery catheter.

In some examples, the twist resolution pocket is fixed on the first elongate shaft or the second elongate shaft.

In some examples, at least one of two catheter shaft channels of the twist resolution pocket slidably receives the first elongate shaft or the second elongate shaft.

In some examples, the first catheter shaft channel slidably receives the first elongate shaft of the first delivery catheter, and the second catheter shaft channel slidably receives the second elongate shaft of the second delivery catheter.

In some examples, a length of the twist resolution pocket is in a range of 5-60 mm (mm). In some examples, a length of the first catheter shaft channel of the twist resolution pocket is in a range of 5-60 mm, and an outer diameter of the first catheter shaft channel is in a range of 0.040-0.050 mm. In some examples, a length of the second catheter shaft channel of the twist resolution pocket is in a range of 5-60 mm, and an outer diameter of the second catheter shaft channel is in a range of 0.030-0.040 mm. In some examples, the twist resolution pocket is positioned at a pocket-to-bifurcation distance in a range of 30-100 mm when the first delivery catheter and the second delivery catheter are advanced distally and pushed against the carina of the bifurcation. In some examples, the twist resolution pocket is positioned at a pocket-to-bifurcation distance in a range of 30-50 mm when the first delivery catheter and the second delivery catheter are advanced distally and pushed against the carina of the bifurcation.

In some examples, a cross-sectional profile of the twist resolution pocket, or a cross-sectional profile of at least one of the two catheter shaft channels, includes an asymmetric or oval shape.

In some examples, a cross-sectional profile of a region of the first elongate shaft or a region of the second elongate shaft pushed against the carina includes an asymmetric or oval shape.

In some examples, the second radially expandable stent comprises a sidewall having a side hole therethrough, and a portion of the first delivery catheter is disposed under a portion of the second radially expandable stent and a portion of the first delivery catheter passes through the side hole in the second radially expandable stent while the second radially expandable stent is in the collapsed configuration, and second delivery catheter is axially slidable relative to the first delivery catheter while the second radially expandable stent is in the collapsed configuration.

In some examples, the first expandable member and the second expandable member are independently expandable of one another.

In some examples, the first expandable member or the second expandable member comprises a balloon.

In some examples, each of the first delivery catheter and the second delivery catheter comprise an inflation lumen.

In some examples, each of the first delivery catheter and the second delivery catheter comprise a guidewire lumen.

In some examples, the second expandable member is axially spaced apart from the first expandable member such that the second expandable member is proximal to the first expandable member.

In some examples, the proximal second expandable member has a cross-sectional profile larger than a cross-sectional profile of the first expandable member.

In some examples, one of the first elongate shaft or the second elongate shaft comprises a region having a guidewire lumen, an inflation lumen, and an exchange lumen, wherein a remaining elongate shaft is slidably disposed in the exchange lumen, and wherein an expandable member on the remaining elongate shaft is axially spaced apart from the first elongate shaft having the exchange lumen such that the expandable member on the remaining shaft is distal to the expandable member on the elongate shaft with the exchange lumen.

In some examples, a method of treating a bifurcated vessel is provided. An example method comprises providing a first delivery catheter and a second delivery catheter, wherein the first delivery catheter comprises a first elongate shaft, a first expandable member, and a first stent disposed over the first expandable member, the first stent having a collapsed configuration and an expanded configuration, in the collapsed configuration the first stent being crimped on the first expandable member with the first expandable member in an uninflated delivery configuration, and in the expanded configuration the first stent being expanded radially by expansion of the first expandable member from the delivery configuration so as to support a vessel wall, and the second delivery catheter comprises a second elongate shaft, a second expandable member, and a second stent disposed over the second expandable member, the second stent having a collapsed configuration and an expanded configuration, in the collapsed configuration the second stent being crimped on the second expandable member with the second expandable member in an uninflated delivery configuration, and in the expanded configuration the second stent being expanded radially by expansion of the second expandable member from the delivery configuration so as to support a vessel wall, and wherein a portion of the first elongate shaft is disposed under the second stent and the first elongate shaft exits a side hole in the second stent, the first expandable member being distal to the second expandable member; advancing both the first delivery catheter and the second delivery catheter through a main branch vessel having a lesion to a bifurcation in the main branch vessel while the first and second stents are in the collapsed configuration, the bifurcation comprising a side branch vessel having a lesion and extending from the main branch vessel, wherein the first stent is advanced into the side branch, distal to the side branch lesion; and providing a twist resolution pocket disposed on the first delivery catheter or the second delivery catheter, wherein the twist resolution pocket is positioned and configured such that when the first delivery catheter and the second delivery catheter are pushed against a carina of the bifurcation during said advancement, twisting in the first delivery catheter or the second delivery catheter is pushed back proximally through the first delivery catheter or the second delivery catheter and away from the bifurcation.

In some examples, the twist resolution pocket includes two catheter shaft channels including a first catheter shaft channel to receive the first elongate shaft of the first delivery catheter, and a second catheter shaft channel to receive the second elongate shaft of the second delivery catheter.

In some examples, the twist resolution pocket is fixed on the first elongate shaft or the second elongate shaft.

In some examples, at least one of two catheter shaft channels of the twist resolution pocket slidably receives the first elongate shaft or the second elongate shaft.

In some examples, the first catheter shaft channel of the twist resolution pocket slidably receives the first elongate shaft of the first delivery catheter, and the second catheter shaft channel of the twist resolution pocket slidably receives the second elongate shaft of the second delivery catheter.

In some examples, a length of the twist resolution pocket is in a range of 5-60 mm. In some examples, a length of the first catheter shaft channel of the twist resolution pocket is in a range of 5-60 mm, and an outer diameter of the first catheter shaft channel is in a range of 0.040-0.050 mm. In some examples, a length of the second catheter shaft channel of the twist resolution pocket is in a range of 5-60 mm, and an outer diameter of the second catheter shaft channel is in a range of 0.030-0.040 mm.

In some examples, the method further comprises positioning the twist resolution pocket at a pocket-to-bifurcation distance in a range of 30-100 mm when the first delivery catheter and the second delivery catheter are advanced distally and pushed against the carina of the bifurcation.

In some examples, the method further comprises positioning the twist resolution pocket at a pocket-to-bifurcation distance in a range of 30-50 mm when the first delivery catheter and the second delivery catheter are advanced distally and pushed against the carina of the bifurcation.

In some examples, a cross-sectional profile of the twist resolution pocket, or a cross-sectional profile of at least one of the two catheter shaft channels, includes an asymmetric or oval shape.

In some examples, a cross-sectional profile of a region of the first elongate shaft or a region of the second elongate shaft pushed against the carina includes an asymmetric or oval shape.

In some examples, the method further comprises proximally retracting the first elongate shaft under a portion of the second stent in the collapsed configuration until a proximal end of the first stent is aligned with the side hole in the second stent; radially expanding the first expandable member, thereby simultaneously expanding the first stent into engagement with the lesion in the side branch vessel and expanding a proximal portion of the second stent in the main branch vessel from the collapsed configuration; and radially expanding the second expandable member, thereby further expanding the proximal portion of the second stent and expanding a distal portion of the second stent into engagement with a wall of the main branch vessel.

In some examples, the advancing comprises advancing both the first and the second delivery catheters until a resistance to further advancement is felt by an operator. In some examples, the resistance is provided by separation of the first elongate shaft from the second elongate shaft as both shafts are advanced against a wall formed between the main branch and the side branch.

In some examples, the first expandable member comprises a balloon, and the expanding of the first expandable member comprises inflating the balloon.

In some examples, the method further comprises contracting the first expandable member after expansion thereof and prior to the expansion of the second expandable member.

In some examples, the expanding of the first stent comprises differentially expanding the first stent so that a proximal region of the expanded first stent has a larger diameter than a distal region of the expanded first stent.

In some examples, the second expandable member comprises a balloon, and the expanding of the second expandable member comprises inflating the balloon. In some examples, the expanding of the second expandable member comprises expanding at least a portion of the second stent into engagement with the main branch lesion.

In some examples, a therapeutic agent is disposed on one or more of the stents, on one or more of the expandable members, on both stent and expandable member, or in any combination or permutation of stent and expandable member.

Side Branch Stenting with Pre-Deployed Main Branch Stent

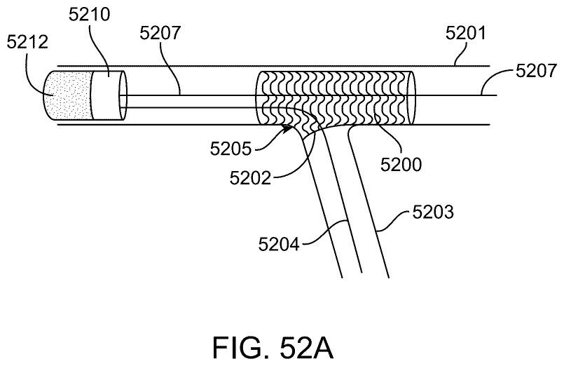

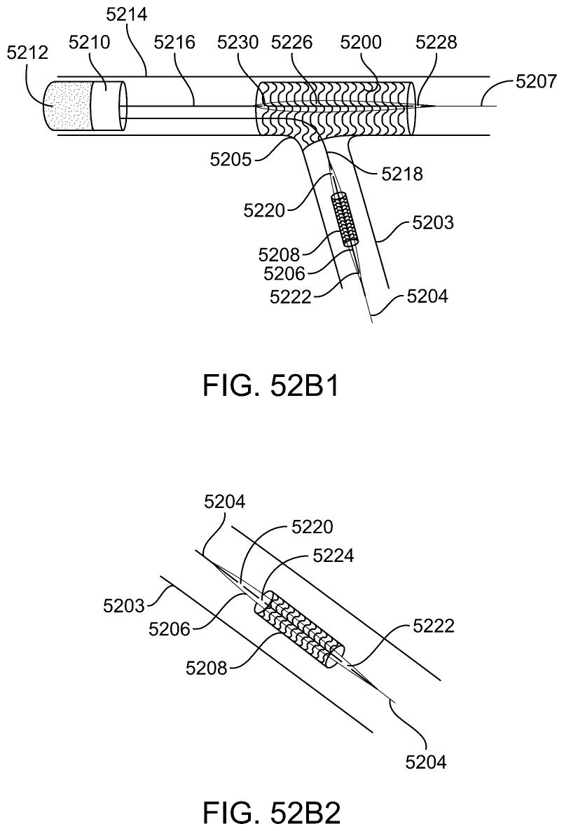

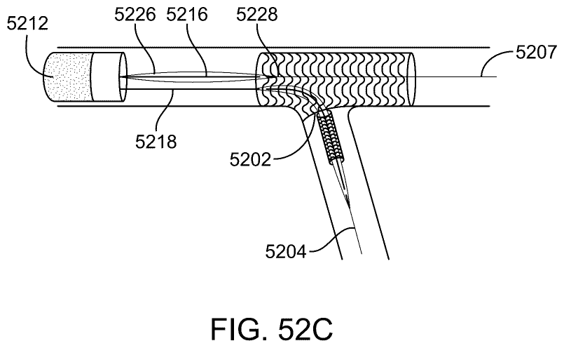

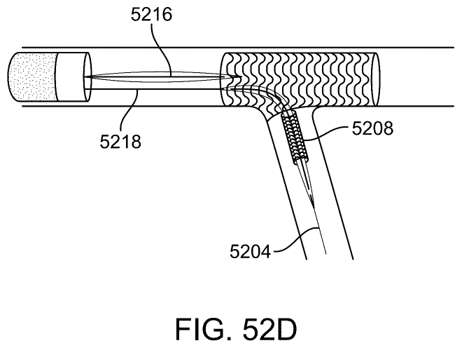

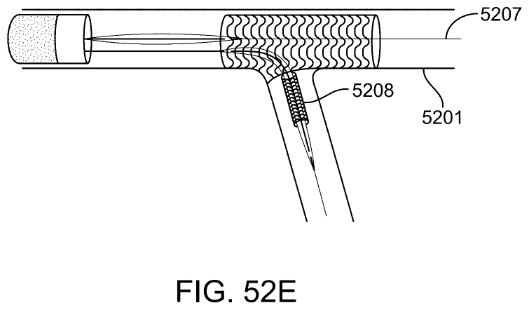

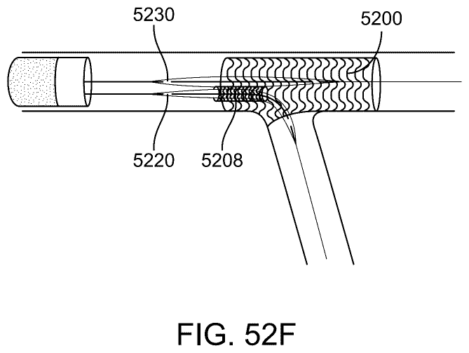

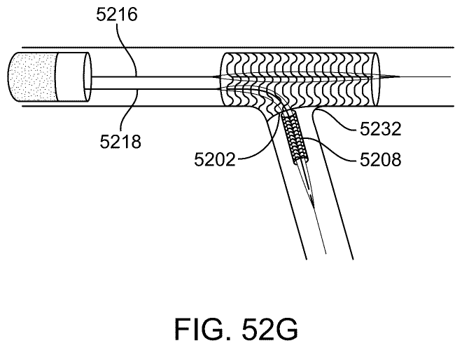

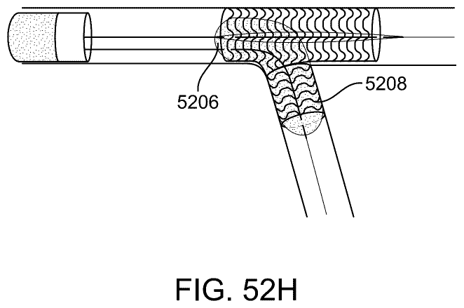

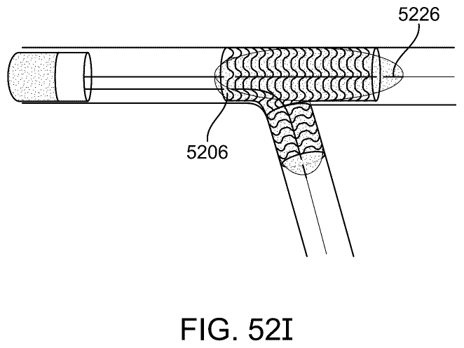

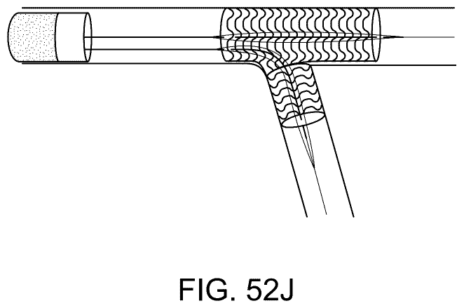

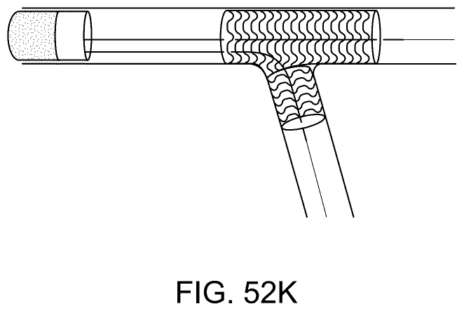

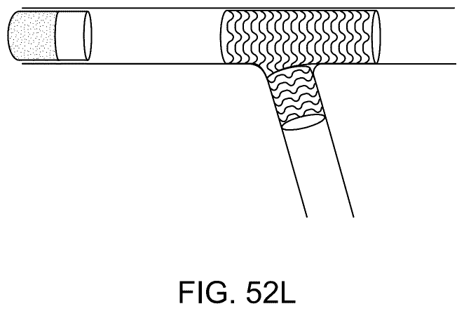

FIGS. 52A-52L illustrate example techniques for side branch stenting, according to some examples. More specifically, FIGS. 52A-52L illustrate various ways a side branch stent can be delivered and deployed through a previously deployed or preexisting main branch stent. These examples are not intended to be limiting, and one of skill in the art will appreciate that other arrangements may be used. Any of the delivery systems and stents according to any of the examples disclosed herein may be used in this example method. Additionally, any of the features described in this specification from any of the devices, systems and methods may be used in combination with or substituted for any of the features disclosed in this example of a treatment method.

FIG. 52A shows a bifurcated vessel having a mother vessel 5201 (the vessel extending horizontally past the bifurcation) and a daughter vessel 5203 (the vessel angulated downwardly after the bifurcation). The mother vessel 5201 may be referred to as the main branch and the daughter vessel 5203 may be referred to as the side branch. A main branch stent 5200 has been pre-deployed at the bifurcation 5205. In some examples, a “pre-deployed” main stent means that the main stent preexists in the main branch at the location of the bifurcation prior to insertion or introduction of any side branch stenting guidewires or catheters at that location. Guidewires or catheters previously used to install the pre-deployed main stent have since been removed. In some examples, a side branch stent 5208 can be deployed as described below to reinforce or repair a bifurcation or treatment zone at which a main branch stent 5200 has previously been deployed. In other examples, the side branch stent may be deployed shortly after the main branch stent has been deployed but some or all of the guidewires and/or catheters previously used to deliver and deploy the pre-deployed main branch stent may be re-used to deliver and deploy the side branch stent.

In some examples, a pre-deployed main branch stent 5200 may previously have been installed at a bifurcation a relatively short period (for example a few hours) before deployment of a side branch stent as described below, but in some cases the main branch stent 5200 may have been pre-deployed several days, weeks, or even months prior to deployment of the side branch stent 5208 in accordance with the side branch stenting techniques discussed below. In relatively longer pre-deployment instances, the main branch stent 5200 may have become embedded or incorporated into the walls of the main branch vessel, for example by reendothelialization.

Structurally in some examples, a pre-deployed main branch stent 5200 may include a preformed side hole 5202, for example as shown in FIG. 52A to allow immediate access to the side branch. As shown in FIG. 52A, (or as described further above in some examples), a side hole 5202 may allow passage of treatment components such a side branch guidewire 5204 (e.g., Wire B in FIG. 52A), and/or an inflatable member (e.g., a side branch balloon 5206 described below), and/or a side branch stent 5208 (e.g., as described further below) into the side branch vessel 5203 through the side hole 5202. These and other components can be deployed in some examples in accordance with the side branch stenting techniques described further below. As described further below, a tip 5210 of a guide catheter 5212 of an example dual catheter delivery system is visible in the view of FIG. 52A.

In some other examples, the pre-deployed main branch stent 5200 does not include a preformed side hole 5202. For example, the main branch stent may be of conventional (no side hole) type, or be sourced from or include a different manufacturer, feature, structure, or material, than a side branch stent subsequently deployed to reinforce a pre-deployed main branch or a treatment location. In the event no side hole is provided or yet exists in the pre-deployed main branch stent, some precursor operations may be performed to establish access to the side branch 5203 to enable the side branch stenting techniques discussed below.

For example, in this regard, a side opening may be formed in a wall of the main branch stent in situ to allow passage of components into the side branch vessel through the thus-formed side opening. In some examples, side branch access may be obtained via an alternate space, gap or aperture identified in the structure of the main branch stent 5200. An alternate, space, gap or aperture may include, for example, an open space in a cell or lattice structure of the main branch stent 5200 formed between adjacent interconnected struts.

In some instances, this alternate space, gap, or aperture may initially be so irregular in shape or of insufficient size to allow entry of a side branch stent or the other treatment component into the side branch. In these instances, the shape and/or size of the alternate gap or aperture may be adjusted or enlarged for example by introducing an unexpanded expandable member such as a balloon, through the space, gap or aperture on a thin guidewire or catheter passed therethrough, and then inflated to adjust the shape or size sufficiently to allow side branch entry of the desired treatment components, as needed. The expandable member may then be deflated and withdrawn.

In any event, once side branch access has been established, whether via a preformed side hole 5202 in the pre-deployed main branch stent 5200, or via an in situ or alternate space, gap, or aperture as discussed above, the side branch guidewire 5204 is inserted into the side branch 5203 (e.g., Wire B in FIG. 52A) and a main branch guidewire 5207 (e.g., Wire A of FIG. 52A) is inserted into the main branch 5201 through the main passage of the pre-deployed main branch stent 5200, as shown. Examples of such guidewires may be 0.014″ guidewires, but this is not intended to be limiting.

In FIG. 52B1, a dual catheter side stent delivery system is deployed over the inserted side branch guidewire 5204 and the main branch guidewire 5207. The delivery system may be any of those described herein. The tip 5210 of a guide catheter 5212 of an example dual catheter delivery system is visible in the views. The guide catheter 5212 guides the two guidewires i.e., the main branch guidewire 5207 and the side branch guidewire 5204, through the upstream pre-bifurcation blood vessel 5214. Beyond (distally) of the bifurcation 5205, the guidewires 5207 and 5204 pass into the main branch 5201 and the side branch 5203, respectively.

In FIG. 52B1, a main branch catheter 5216 is deployed over the main branch guidewire 5207. The main branch catheter 5216 may include a main branch balloon 5226, a proximal main branch marker 5230 disposed on the elongate shaft of the main branch catheter and positioned at the proximal end of the working length of the balloon, and a distal main branch radiopaque marker 5228 similarly disposed on the main branch catheter shaft and positioned at the distal end of the working length of the balloon. Thus, the two markers indicate the proximal and distal ends of the working length of the balloon. A side branch catheter 5218 is deployed over the side branch guidewire 5204. Note, in some examples of the present side branch stenting techniques, only the side branch catheter 5218 carries a stent (as described for example further below) as the main branch stent has already been pre-deployed, as noted further above.

As further shown in FIG. 52B1, the side branch catheter 5218 may include the side branch balloon 5206 and one or more radiopaque markers, such as a proximal side branch radiopaque marker 5220 and a distal side branch radiopaque marker 5222. The markers may be disposed on the elongate shaft of the side branch catheter and they may be positioned at the proximal and distal working ends of the side branch balloon. The side branch catheter 5218 carries an example side branch stent 5208 for deployment at the bifurcation 5205 in accordance with the procedures discussed below, or in accordance with any one or more of the stents, catheters, stent deployment and/or bifurcation treatment procedures described further above.

In FIG. 52B1, the side branch stent 5208 is shown as a full stent covering at least a full midsection of the side branch balloon 5206. In some examples, a full side branch stent covers substantially the entire length of the working length of the side branch balloon 5206, or at least extends between the proximal radiopaque marker 5220 and the distal radiopaque marker 5222. In the latter instance, the radiopaque markers 5220 and 5222 may thus indicate proximal and distal ends of the side branch stent 5208, as well as proximal and distal locations on the side branch balloon 5206, in some examples.

In another side branch stenting example shown in FIG. 52B2, the side branch stent 5208 is shown as a half stent covering only a distal portion of the side branch balloon 5206. In further examples, a full or half side branch stent 5208 may be positioned on other portions of the side branch balloon 5206. Additionally, as shown in FIG. 52B2, a third radiopaque marker 5224 may be provided in an intermediate portion of the side branch balloon, which may be in the middle of the side branch catheter portion supporting the side branch balloon 5206. In this example, optionally the stent is disposed over a distal portion of the side branch balloon while a proximal portion of the balloon remains uncovered by the stent. Thus, various arrangements of the components illustrated in FIGS. 52B1-52B2 are possible.

In further examples, for instance where the side branch has a stenotic lesion, it may be advantageous to treat the side branch stent with a drug coated balloon, where the drug (also referred to herein as a therapeutic agent) pharmacologically reduces or eliminates the stenotic lesion as opposed to the simple mechanical reduction in stenosis provided by POBA (plain old balloon angioplasty), and the subsequent mechanical support provided by a stent. Additional information related to drug coated balloons is disclosed in U.S. Provisional patent application Ser. No. xx/xxx,xxx, filed on July xx, 2024, the entire contents of which are incorporate herein by reference.

In FIG. 52C, the dual catheter delivery system is pulled back proximally (the main branch catheter 5216 and the side branch catheter 5218 are simultaneously pulled back proximally) so that the main branch catheter 5216 is withdrawn behind the side hole 5202. In some examples, this position may be denoted by the distal main branch radiopaque marker 5228 lying adjacent the proximal side of the side hole 5202.

In FIG. 52D, the main branch guidewire 5207 is additionally pulled back in some examples to mitigate potential wire crossing. Further techniques to address and/or mitigate wire crossing are described herein with reference to FIGS. 49A-49D above.

In FIG. 52E, the main branch guidewire 5207 is advanced distally to be placed back into the main branch 5201 with the distal tip of the guidewire extending through the main branch stent and extending past the distal end of the main branch stent.

In FIG. 52F, the proximal markers 5230 and 5220 are aligned. More specifically, the proximal main branch marker 5230 and the proximal side branch marker 5220 are aligned at a position proximal of the pre-deployed main branch stent 5200. This position may be at a distance of 1 cm proximally of the pre-deployed main branch stent in some examples.

In FIG. 52G, the dual catheter delivery system is advanced distally toward the carina 5232 of the bifurcation until some resistance or tension is felt by an operator. In some examples, the catheters 5216 and 5218 diverge either side of the carina 5230 without necessarily touching the carina. The side branch stent 5208 is advanced though the side hole 5202. Some examples may include a proximal retraction of the side branch catheter 5218 into the side hole 5202 of the main branch stent.

Here, the proximal radiopaque marker 5220 on the side branch catheter 5218 may be axially aligned with the proximal radiopaque marker 5230 of the main catheter 5216. A proximal portion of the side branch balloon 5206 may be disposed under a proximal portion of the main branch stent, and a distal portion of the side branch balloon 5206 may be disposed outside the side hole 5202. This portion of the side branch balloon 5206 may include a drug coating since in some cases it may be beneficial not to retract the drug coated portion of the side branch balloon through the side hole of the main branch stent and into the main branch stent since the main branch stent can abrade the drug coating and damage the coating or remove it from the side branch balloon. Once the radiopaque markers 5220 and 5230 are aligned, the axial position of both the main branch and side branch catheters may be maintained relative to one another.

In some examples, there is no contact between the delivery system and the carina, while in other examples there may be contact. The side hole 5202 in the main branch stent 5200 may also be oriented so that it is adjacent the ostium to the side branch, and the distal end of the main branch stent 5200 may be distal of the bifurcation depending on the length of the main branch stent. The proximal end of the main branch stent 5200 may be proximal of the ostium to the side branch.

In FIG. 52H, the side branch balloon 5206 is inflated to expand the side branch stent 5208. The expanded side branch balloon 5206 abuts the walls of the side branch from the ostium distally into the side branch. The proximal portion of the side branch balloon 5206 may also expand under the main branch stent 5200, expanding the proximal portion of the main branch stent that is proximal of the side hole 5202. The side hole 5202 may also be expanded so that the side hole 5202 aligns and conforms with the ostium to the side branch vessel.

Inflation times and pressure may be varied according to operator preference, for example inflation time may be for 15 to 30 seconds. In another example, inflation pressure may not exceed a range of 8 atmospheres of pressure. Thus, mechanical pressure applied to a stenotic side branch lesion may help reduce the stenosis by compressing the lesion into the vessel walls and the therapeutic agent will also help prevent restenosis.

FIG. 52I illustrates kissing balloons where the main branch balloon 5226 is also inflated while the side branch balloon 5206 remains inflated. This may ensure even expansion of the entire side branch stent (and even reinforcement of the main branch stent) so that they conform to the respective main and side branch vessels and the ostium of the side branch. The even expansion and reinforcement also seek to ensure even compression of the lesion to avoid plaque shifting. Again, inflation times and pressures for the main branch balloon may be varied according to operator preference. In one example, inflation time is 15 to 30 seconds, and inflation pressure does not exceed a range of 8 atmospheres of pressure.

FIG. 52J shows deflation of both the main branch balloon and the side branch balloon. The balloons may be deflated after an adequate time, for example an adequate for a drug to elute from the side branch balloon into the side branch vessel. The drug helps to prevent restenosis after the procedure. Deflation of the balloons may be performed simultaneously or one after the other. The proximal end of the side branch stent abuts the side hole in the main branch stent, to minimize any unstented gaps therebetween so that even support is provided to the vessel walls. The proximal end of the side branch stent may interdigitate with the side hole as previously disclosed above in FIGS. 47A-47D.

FIG. 52K shows proximal retraction of the delivery system through the guide catheter 5212 and out the patient. Proximal retraction may be performed simultaneously or one catheter after the other. In some examples, the side branch catheter may be proximally retracted into the guide catheter 5212 followed by proximally retracting the main branch catheter into the guide catheter 5212. This helps ensure that both catheters can be easily removed from the patient, and then both the guide catheter and both main branch and side branch catheters can be removed from the patient simultaneously as one unit. Of course, this is not intended to be limiting and the operator may retract and remove the balloon catheters and guide catheters in any order or any desired manner.

FIG. 52L shows a final step where both guidewires are retracted proximally and removed from the patient either through the guide catheter if it is still in the patient, or by proximally retracting both guide wires through the vasculature if the guide catheter has already been removed. This leaves only the pre-deployed main branch stent in the main branch, and the subsequently deployed side branch stent in the side branch vessel.

FIGS. 52A-52L thus illustrate the use of a dual catheter delivery system disclosed herein to perform example side branch stenting techniques. The disclosed examples are not intended to be limiting. The example of a technique seeks to ensure that the stents are properly aligned and deployed without causing damage to the pre-existing main branch stent and provides a smooth lumen for blood flow through the bifurcation.

### Therapeutic Agent

In any of the examples discussed herein, a therapeutic agent may be disposed on one or more of the stents, on one or more of the balloons, on both stent and balloon or in any combination or permutation of stents and balloons. Some examples include a therapeutic agent on the mother balloon only, on the daughter balloon only, or on both the mother and daughter balloons. The therapeutic agent may be on the mother stent only, the daughter stent only, or on both the mother stent and the daughter stent. The therapeutic agent may be on the mother stent and the mother balloon, on the daughter stent and the daughter balloon, on the mother stent and the daughter balloon, or on the mother balloon and on the daughter stent. The drug may be eluted from the stent or balloon in a controlled manner into the target treatment area such as a stenotic lesion. Examples of therapeutic agents may include those that help inhibit restenosis, hyperplasia or have other therapeutic benefits. Examples of anti-hyperplasia agents include anti-neoplastic drugs, such as paclitaxel, methotrexate, and batimastal; antibiotics such as doxycycline, tetracycline, rapamycin, everolimus, biolimus A9, novolimus, myolimus, zotarolimus, and other analogs and derivatives of rapamycin, and actinomycin; immuni suppressants such as dexamethasone and methyl prednisolone; nitric oxide sources such as nitroprussides; estrogen; estradiols; and the like. Methods for applying the therapeutic agent to the stent or balloon are well known to those skilled in the art and have been described in the patent and scientific literature. Notes And Examples

The following, non-limiting examples, detail certain aspects of the present subject matter to solve the challenges and provide the benefits discussed herein, among others.

The above detailed description includes references to the accompanying drawings, which form a part of the detailed description. The drawings show, by way of illustration, specific examples in which the invention can be practiced. These examples are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other examples can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed example. Thus, the following claims are hereby incorporated into the Detailed Description as examples or examples, with each claim standing on its own as a separate example, and it is contemplated that such examples can be combined with each other in various combinations or permutations. The scope of the invention should be determined with reference to the appended claims, along with the full scope of equivalents to which such claims are entitled.

### EXAMPLES

Example 1 includes a method of treating a bifurcated vessel having a pre-deployed main branch stent located at a bifurcation in the bifurcated vessel, the method comprising: providing a first delivery catheter and a second delivery catheter, wherein the first delivery catheter comprises a first elongate shaft, a first expandable member, and a first stent disposed over the first expandable member, the first stent having a collapsed configuration and an expanded configuration, and the second delivery catheter comprises a second elongate shaft, and a second expandable member; advancing both the first delivery catheter and the second delivery catheter through a main branch vessel to the pre-deployed main branch stent located at the bifurcation while the first stent is in the collapsed configuration, the bifurcation comprising a side branch vessel extending from the main branch vessel; advancing the second elongate shaft distally through a main lumen of the pre-deployed main branch stent; advancing the first elongate shaft distally to introduce the first stent into the side branch vessel through a side hole in the pre-deployed main branch stent; and expanding the first expandable member to expand the first stent into contact with a wall of the side branch vessel.

Example 2 includes the method of example 1, wherein in the collapsed configuration the first stent is crimped on the first expandable member with the first expandable member in an uninflated delivery configuration, and in the expanded configuration the first stent is expanded radially by expansion of the first expandable member from the collapsed configuration so as to abut and support the wall of the side branch vessel.

Example 3 includes the method of example 1 or example 2, wherein after expansion of the first stent a portion of the first elongate shaft is disposed under the pre-deployed main branch stent and the first elongate shaft exits the side hole in the pre-deployed main branch stent.

Example 4 includes the method of any one of examples 1 to 3, further comprising axially aligning respective proximal markers on the first delivery catheter and the second delivery catheter before expanding the first stent into contact with the wall of the side branch vessel.

Example 5 includes the method of any one of examples 1 to 4, wherein at least a portion of the first expandable member lies adjacent at least a portion of the second expandable member when the respective proximal markers are aligned.

Example 6 includes the method of any one of examples 1-5, wherein advancing the first elongate shaft distally to introduce the first stent into the side branch vessel through the side hole in the pre-deployed main branch stent includes advancing the first stent fully distal to the side hole or a lesion at the bifurcation.

Example 7 includes the method of any one of examples 1 to 6, further comprising retracting the first stent into the side hole in the pre-deployed main branch stent before expanding the first stent into contact with the wall of the side branch vessel.

Example 8 includes the method of any one of examples 1 to 7, wherein the side hole is a preformed side hole in the pre-deployed main branch stent.

Example 9 includes the method of any one of examples 1 to 8, wherein the side hole is formed in the pre-deployed main branch stent in situ prior to advancing both the first delivery catheter and the second delivery catheter through the main branch vessel to the pre-deployed main branch stent.

Example 10 includes the method of any one of examples 1 to 9, wherein the side hole includes a space, gap or aperture defined by a cell or lattice structure of the pre-deployed main branch stent.

Example 11 includes the method of any one of examples 1 to 10, further comprising: proximally retracting the first elongate shaft under a portion of the pre-deployed main branch stent until a proximal end of the first stent is aligned with the side hole in the pre-deployed main branch stent; and radially expanding the first expandable member, thereby simultaneously expanding the first stent into engagement with a wall of the side branch vessel and tacking a proximal portion of the pre-deployed main branch stent in the main branch vessel.

Example 12 includes the method of any one of examples 1 to 11, further comprising: radially expanding the second expandable member, thereby further tacking the proximal portion of the pre-deployed main branch stent and tacking a distal portion of the pre-deployed main branch stent in engagement with a wall of the main branch vessel.

Example 13 includes the method of any one of examples 1 to 12, wherein advancing both the first delivery catheter and the second delivery catheter comprises advancing both the first delivery catheter and the second delivery catheter until a resistance to further advancement is felt by an operator.

Example 14 includes the method of any one of examples 1 to 13, wherein the resistance is provided by separation of the first elongate shaft from the second elongate shaft as both shafts are advanced respectively into the main branch vessel and the side branch vessel.

Example 15 includes the method of any one of examples 1 to 14, wherein the first expandable member comprises a balloon, and expanding of the first expandable member comprises inflating the balloon.

Example 16 includes the method of any one of examples 1 to 12, further comprising maintaining expansion of the first expandable member after expansion thereof and prior to the expansion of the second expandable member.

Example 17 includes the method of any one of examples 1 to 12, wherein the second expandable member comprises a balloon, and expanding of the second expandable member comprises inflating the balloon.

Example 18 includes the method of any one of examples 1 to 17, wherein a therapeutic agent is disposed on the first stent, or on one or more of the first expandable member and the second expandable member, or on both the first stent and an expandable member, or in any combination or permutation of stent and expandable member.

Example 19 includes the method of any one of examples 1 to 18, wherein the therapeutic agent is one or more of an anti-platelet agent, anti-inflammatory agent, anti-hyperlipidemic agent, anti-proliferative agent, an antibiotic, or an anti-thrombogenic agent, or wherein the therapeutic agent comprises paclitaxel.

Examples 20 includes the method of any one of examples 1 to 19, wherein the first delivery catheter further comprises a proximal marker, a distal marker, and an intermediate marker, each disposed on the first elongate shaft, the proximal marker, the distal marker, and the intermediate marker being configured to be observed under an imaging modality, wherein the proximal marker is axially aligned with a proximal portion of a working length of the first expandable member, wherein the distal marker is axially aligned with a distal portion of the working length of the first expandable member, and wherein the intermediate marker is disposed between the proximal marker and the distal marker, and the intermediate marker is axially aligned with an end of the first stent.

Example 21 includes a method for deploying a stent in a bifurcated vessel having a pre-existing main branch stent, comprising: inserting a main branch guide wire and a side branch guidewire respectively into a main branch and a side branch of the bifurcated vessel; advancing a dual catheter delivery system over the guide wires, the dual catheter delivery system comprising a main branch catheter and a side branch catheter, wherein only the side branch catheter carries a stent for the side branch; retracting the delivery system to position the main branch catheter behind a side hole of the pre-existing main branch stent; pulling back the main branch guide wire to reduce potential wire crossing; repositioning the main branch guide wire into the main branch; aligning proximal marker bands on the catheters to ensure correct positioning of the side branch stent relative to the side hole; advancing the delivery system towards a carina of the bifurcation until a tension is felt by an operator; passing at least a portion of the side branch stent through the side hole of the pre-existing main branch stent; inflating a balloon on the side branch catheter to expand the side branch stent into contact with a wall of the side branch; performing a kissing balloon inflation with the balloon on the side branch catheter and a balloon on the main branch catheter; deflating the balloons and removing both catheters from the vessel; and removing the guide wires, leaving the pre-existing main branch stent and the side branch stent aligned at the bifurcation.

Example 22 includes the method of example 21, wherein the side branch stent is deployed through a preformed side hole in the pre-existing main branch stent.

Example 23 includes the method of example 21 or example 22, wherein the side branch stent deployment includes expanding the side branch stent to contact walls of the side branch vessel and the pre-existing main branch stent.

Example 24 includes the method of any one of examples 21 to 23, further comprising using radiopaque markers on the catheters to facilitate visual alignment under imaging during the procedure.

Example 25 includes the method of any one of examples 21 to 24, wherein the kissing balloon inflation ensures that the deployed side branch stent and the pre-existing main branch stent are fully apposed to the vessel walls and each other.

Example 26 includes the method of any one of examples 21 to 25, wherein the guide wires are 0.014″ guidewires.

Example 27 includes the method of any one of examples 21 to 26, wherein the dual catheter delivery system includes a guidewire for the main branch and a guidewire for the side branch to facilitate differentiated manipulation and control during a procedure.

Example 28 includes the method of any one of examples 21 to 27, wherein the tension felt during advancement of the delivery system is used as a feedback mechanism by the operator to mitigate over-advancement and potential damage to the bifurcation.

Example 29 includes the method of any one of examples 21 to 28, wherein the deflation and removal of the catheters are performed simultaneously to maintain the alignment and position of the stents during withdrawal.

Example 30 includes the method of any one of examples 21 to 29, further comprising confirming an alignment and deployment of the stents using an imaging technique post-procedure to ensure optimal placement and function.

In Example 31, the systems, apparatuses, or methods of any one or any combination of Examples 1 to 30 can optionally be configured such that all elements or options recited are available to use or select from.

While the above is a complete description of some example of the inventive subject matter, various alternatives, modifications, and equivalents may be used. Therefore, the above description should not be taken as limiting the scope of the inventive subject matter which is defined by the appended claims.

What is claimed is:

1. A method of treating a bifurcated vessel having a pre-deployed main branch stent located at a bifurcation in the bifurcated vessel, the method comprising:
providing a first delivery catheter and a second delivery catheter, wherein the first delivery catheter comprises a first elongate shaft, a first expandable member, and a first stent disposed over the first expandable member, the first stent having a collapsed configuration and an expanded configuration, and the second delivery catheter comprises a second elongate shaft, and a second expandable member;
advancing both the first delivery catheter and the second delivery catheter through a main branch vessel to the pre-deployed main branch stent located at the bifurcation while the first stent is in the collapsed configuration, the bifurcation comprising a side branch vessel extending from the main branch vessel;
advancing the second elongate shaft distally through a main lumen of the pre-deployed main branch stent;
advancing the first elongate shaft distally to introduce the first stent into the side branch vessel through a side hole in the pre-deployed main branch stent; and
expanding the first expandable member to expand the first stent into contact with a wall of the side branch vessel.

2. The method of claim 1, wherein in the collapsed configuration the first stent is crimped on the first expandable member with the first expandable member in an uninflated delivery configuration, and in the expanded configuration the first stent is expanded radially by expansion of the first expandable member from the collapsed configuration so as to abut and support the wall of the side branch vessel.

3. The method of claim 2, wherein after expansion of the first stent a portion of the first elongate shaft is disposed under the pre-deployed main branch stent and the first elongate shaft exits the side hole in the pre-deployed main branch stent.

4. The method of claim 3, further comprising axially aligning respective proximal markers on the first delivery catheter and the second delivery catheter before expanding the first stent into contact with the wall of the side branch vessel.

5. The method of claim 4, wherein at least a portion of the first expandable member lies adjacent at least a portion of the second expandable member when the respective proximal markers are aligned.

6. The method of claim 5, wherein advancing the first elongate shaft distally to introduce the first stent into the side branch vessel through the side hole in the pre-deployed main branch stent includes advancing the first stent fully distal to the side hole or a lesion at the bifurcation.

7. The method of claim 6, further comprising retracting the first stent into the side hole in the pre-deployed main branch stent before expanding the first stent into contact with the wall of the side branch vessel.

8. The method of claim 1, wherein the side hole is a preformed side hole in the pre-deployed main branch stent.

9. The method of claim 1, wherein the side hole is formed in the pre-deployed main branch stent in situ prior to advancing both the first delivery catheter and the second delivery catheter through the main branch vessel to the pre-deployed main branch stent.

10. The method of claim 1, wherein the side hole includes a space, gap or aperture defined by a cell or lattice structure of the pre-deployed main branch stent.

11. The method of claim 1, further comprising:
proximally retracting the first elongate shaft under a portion of the pre-deployed main branch stent until a proximal end of the first stent is aligned with the side hole in the pre-deployed main branch stent; and
radially expanding the first expandable member, thereby simultaneously expanding the first stent into engagement with a wall of the side branch vessel and tacking a proximal portion of the pre-deployed main branch stent in the main branch vessel.

12. The method of claim 11, further comprising:
radially expanding the second expandable member, thereby further tacking the proximal portion of the pre-deployed main branch stent and tacking a distal portion of the pre-deployed main branch stent in engagement with a wall of the main branch vessel.

13. The method of claim 1, wherein advancing both the first delivery catheter and the second delivery catheter comprises advancing both the first delivery catheter and the second delivery catheter until a resistance to further advancement is felt by an operator.

14. The method of claim 13, wherein the resistance is provided by separation of the first elongate shaft from the second elongate shaft as both shafts are advanced respectively into the main branch vessel and the side branch vessel.

15. The method of claim 1, wherein the first expandable member comprises a balloon, and expanding of the first expandable member comprises inflating the balloon.

16. The method of claim 12, further comprising maintaining expansion of the first expandable member after expansion thereof and prior to the expansion of the second expandable member.

17. The method of claim 12, wherein the second expandable member comprises a balloon, and expanding of the second expandable member comprises inflating the balloon.

18. The method of claim 1, wherein a therapeutic agent is disposed on the first stent, or on one or more of the first expandable member and the second expandable member, or on both the first stent and an expandable member, or in any combination or permutation of stent and expandable member.

19. The method of claim 18, wherein the therapeutic agent is one or more of an anti-platelet agent, anti-inflammatory agent, anti-hyperlipidemic agent, anti-proliferative agent, an antibiotic, or an anti-thrombogenic agent, or wherein the therapeutic agent comprises paclitaxel.

20. The method of claim 1, wherein the first delivery catheter further comprises a proximal marker, a distal marker, and an intermediate marker, each disposed on the first elongate shaft, the proximal marker, the distal marker, and the intermediate marker being configured to be observed under an imaging modality,
wherein the proximal marker is axially aligned with a proximal portion of a working length of the first expandable member,
wherein the distal marker is axially aligned with a distal portion of the working length of the first expandable member, and
wherein the intermediate marker is disposed between the proximal marker and the distal marker, and the intermediate marker is axially aligned with an end of the first stent.

21. A method for deploying a stent in a bifurcated vessel having a pre-existing main branch stent, comprising:
inserting a main branch guide wire and a side branch guidewire respectively into a main branch and a side branch of the bifurcated vessel;
advancing a dual catheter delivery system over the guide wires, the dual catheter delivery system comprising a main branch catheter and a side branch catheter, wherein only the side branch catheter carries a stent for the side branch;
retracting the delivery system to position the main branch catheter behind a side hole of the pre-existing main branch stent;
pulling back the main branch guide wire to reduce potential wire crossing;
repositioning the main branch guide wire into the main branch;
aligning proximal marker bands on the catheters to ensure correct positioning of the side branch stent relative to the side hole;
advancing the delivery system towards a carina of the bifurcation until a tension is felt by an operator;
passing at least a portion of the side branch stent through the side hole of the pre-existing main branch stent;
inflating a balloon on the side branch catheter to expand the side branch stent into contact with a wall of the side branch;
performing a kissing balloon inflation with the balloon on the side branch catheter and a balloon on the main branch catheter;
deflating the balloons and removing both catheters from the vessel; and
removing the guide wires, leaving the pre-existing main branch stent and the side branch stent aligned at the bifurcation.

22. The method of claim 21, wherein the side branch stent is deployed through a preformed side hole in the pre-existing main branch stent.

23. The method of claim 21, wherein the side branch stent deployment includes expanding the side branch stent to contact walls of the side branch vessel and the pre-existing main branch stent.

24. The method of claim 21, further comprising using radiopaque markers on the catheters to facilitate visual alignment under imaging during the procedure.

25. The method of claim 21, wherein the kissing balloon inflation ensures that the deployed side branch stent and the pre-existing main branch stent are fully apposed to the vessel walls and each other.

26. The method of claim 21, wherein the guide wires are 0.014″ guidewires.

27. The method of claim 21, wherein the dual catheter delivery system includes a guidewire for the main branch and a guidewire for the side branch to facilitate differentiated manipulation and control during a procedure.

28. The method of claim 21, wherein the tension felt during advancement of the delivery system is used as a feedback mechanism by the operator to mitigate over-advancement and potential damage to the bifurcation.

29. The method of claim 21, wherein the deflation and removal of the catheters are performed simultaneously to maintain the alignment and position of the stents during withdrawal.

30. The method of claim 21, further comprising confirming an alignment and deployment of the stents using an imaging technique post-procedure to ensure optimal placement and function.
